(19) United States
(12) Patent Application Publication
(10) Pub. No.: US 2011/0245528 A1
(43) Pub. Date: Oct. 6, 2011

(54) THERAPEUTIC COMPOUNDS

(76) Inventor: C. Eric Schwartz, Wakefield, MA (US)

(21) Appl. No.: 13/120,621

(22) PCT Filed: Sep. 23, 2009
(86) PCT No.: PCT/US09/58021

§ 371 Date: Jun. 21, 2011

(30) Foreign Application Priority Data
Sep. 23, 2008 (US) 61/194094

(51) Int. Cl. C07C 69/732; C07C 59/48; C07C 59/46; C07C 59/42

(52) U.S. Cl. 560/60; 562/470; 562/506; 562/587; 562/508; 560/183; 560/126

(57) ABSTRACT

The invention relates to novel resolvin compounds and pharmaceutical preparations thereof. The invention further relates to methods of treatment using the novel resolvin compounds of the invention.

### RELATED APPLICATIONS

This application claims the benefit of priority to U.S. Provisional Patent Application No. 61/194,093, filed Sep. 23, 2008, which application is hereby incorporated by reference in its entirety.

### BACKGROUND

Supplementation of dietary omega-3 polyunsaturated fatty acids (“ω-3 PUFAs”) such as eicosapentaenoic acid, a component of fish oils, may have beneficial effects in diseases such as arteriosclerosis, arthritis, asthma and cancer, which may be mediated by antithrombotic, immunoregulatory and anti-inflammatory responses [De Caterina, R., S. Endres, S. D. Kristensen, and E. B. Schmidt, editors. (1993). n-3 Fatty Acids and Vascular Disease. Springer-Verlag, London; Lands, W. E. M., editor. (1987). Proceedings of the AOCS Short Course on Polyunsaturated Fatty Acids and Eicosanoids. American Oil Chemists' Society, Champaign, Ill.; Iigo, M. et al. (1997) Br. J. Cancer 75:650]. The potential of ω-3 PUFAs for preventative actions in cardiovascular diseases was recently supported by the finding that major dietary ω-3 PUFAs, such as eicosapentaenoic acid (C20:5 ω-3; EPA) and docosahexaenoic acid (C22:6 ω-3; DHA), have a dramatic effect on ischemia-induced ventricular fibrillation [Billman, G. E. et al. (1999) Circulation. 99:2452]. Emergence of such possible preventative and/or therapeutic actions of ω-3 PUFA supplementation in infant nutrition, cardiovascular diseases, and mental health has led to a call for recommended dietary intakes by an international workshop [Simopoulous, A. P. et al. (1999). J. Am. Coll. Nutr. 18:487]. The Gruppo Italiano per lo Studio della Sopravvivense nell'Infarto Miocardio (GISSI) Prevenzione trial evaluated the effects of ω-3 PUFA supplementation with 11,300 patients surviving myocardial infarction taking ˜1 g of ω-3 PUFA daily (n=2,836) along with recommended preventive treatments including aspirin, and reported a significant benefit with a decrease in cardiovascular death [Marchioloi, R. et al. (1999). Lancet. 354:447]. However, the mechanisms underlying the protective action of dietary ω-3 PUFAs in these studies and other studies including those concerned with diseases of the skin, bowel, and neural tissues are not currently understood. One of the many hypothesized elements of the mechanism(s) of action of ω-3 PUFAs is that naturally occurring metabolites, formed from these PUFAs, may act as mediators that provide important biological functions, but these metabolites may have relatively short half-lives in vivo. There remains a need for new analogues which may have greater in vivo stability than naturally occurring ω-3 PUFA metabolites for settings where a longer half-life may be advantageous.

### SUMMARY OF INVENTION

The present invention provides a compound of formula I,

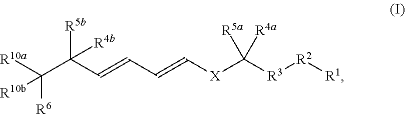

or a pharmaceutically acceptable salt thereof, wherein:
X is selected from —C≡C—, —C(R⁷)═C(R⁷)—, -(cyclopropyl)-, -(cyclobutyl)-, -(cyclopentyl)-, and -(cyclohexyl)-;
R¹ is selected from —OR^{a}, —N(R^{a})—SO₂—R^{c} and —N(R^{a})(R^{b}), wherein each of R^{a} and R^{b} is independently selected from H, C₁-C₆-alkyl, aryl, aralkyl, heteroaryl, and heteroaralkyl, and R^{c} is selected from C₁-C₆-alkyl, aryl, aralkyl, heteroaryl, and heteroaralkyl;
R² is selected from —CH₂—, —C(O)—, —SO₂—, —PO(OR)—, and tetrazole;
R is selected from hydrogen and alkyl;
R³ is selected from a carbocyclic ring, a heterocyclic ring, —(CH₂)ₙ—, CH₂C(O)CH₂, and —CH₂—O—CH₂, wherein:
   n is an integer from 1 to 3;
   any hydrogen atom in R³ is optionally and independently replaced by halo, (C₁-C₅)-alkyl, perfluoroalkyl, aryl, heteroaryl, hydroxy, or O—(C₁-C₅)-alkyl; and
   any two hydrogen atoms bound to a common carbon atom in R³ are optionally taken together with the carbon atom to which they are bound to form a carbocyclic or heterocyclic ring;
each of R^{4a} and R^{4b} is independently selected from hydrogen, halo, —OH, —O—(C₁-C₅)-alkyl, —O-aryl, O-heteroaryl, —O—C(O)—(C₁-C₅)-alkyl, —O—C(O)-aryl, —O—C(O)-heteroaryl, —O—C(O)—O—(C₁-C₅)-alkyl, —O—C(O)—O-aryl, —O—C(O)—O-heteroaryl, and —O—C(O)—N(R^{a})(R^{b}), wherein any alkyl, aryl or heteroaryl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester, thioacyl, thioether, amino, amido, acylamino, cyano, and nitro;
each of R^{5a} and R^{5b} is independently selected from hydrogen, halo, (C₁-C₅)-alkyl, perfluoroalkyl, aryl, and heteroaryl, preferably hydrogen, halo and (C₁-C₅)-alkyl;
R⁶ is selected from -phenyl, —(C₁-C₅)-alkyl, —(C₃-C₇)-cycloalkyl, —C≡C-phenyl, —C≡C—(C₃-C₇)-cycloalkyl, —C≡C—(C₁-C₅)-alkyl, and —O-phenyl, wherein phenyl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester, thioacyl, thioether, amino, amido, acylamino, cyano, and nitro;
   and R⁶ is additionally selected from —C≡CH when:
      a) X is —C(R⁷)═C(R⁷)— or -(cyclopropyl)-; or
      b) each of R^{4a} and R^{4b} is hydrogen or halo; or
      c) each of R^{5a} and R^{5b} is halo; or
      d) R² is —CH₂—;
each R⁷ is independently selected from hydrogen and (C₁-C₅)-alkyl, or two occurrences of R⁷ may optionally be taken together with the carbons to which they are attached to form a 5- or 6-membered ring;
each of R^{10a} and R^{10b} is independently selected from hydrogen, (C₁-C₅)-alkyl, perfluoroalkyl, O—(C₁-C₅)-alkyl, aryl and heteroaryl, or
   R^{10a} and R^{10b} are taken together with the carbon atom to which they are bound to form a carbocyclic or heterocyclic ring;
      and each double bond is independently in an E- or a Z-configuration.

The present invention provides a compound of the formula II,

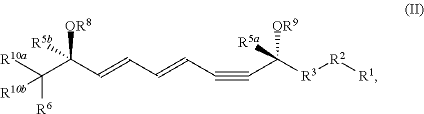

or formula III,

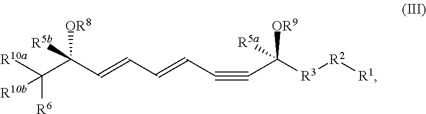

or a pharmaceutically
acceptable salt of either of the foregoing, wherein:
R¹ is selected from —OR^{a}, —N(R^{a})—SO₂—R^{c} and —N(R^{a})(R^{b}), wherein each of R^{a} and R^{b} is independently selected from H, C₁-C₆-alkyl, aryl, aralkyl, heteroaryl, and heteroaralkyl, and R^{c} is selected from C₁-C₆-alkyl, aryl, aralkyl, heteroaryl, and heteroaralkyl;
R² is selected from —C(O)—, —SO₂—, —PO(OR)—, and tetrazole;
R is selected from hydrogen and alkyl;
R³ is selected from —(CH₂)ₙ— and —CH₂—O—CH₂, wherein n is an integer from 1 to 3; and optionally up to two hydrogen atoms in R³ are independently replaced by halo, (C₁-C₅)-alkyl, or O—(C₁-C₅)-alkyl;
each of R^{5a} and R^{5b} is independently selected from hydrogen, (C₁-C₅)-alkyl, perfluoroalkyl, aryl, and heteroaryl, preferably hydrogen and (C₁-C₅)-alkyl;
R⁶ is selected from —C≡CH, -phenyl, —(C₁-C₅)-alkyl, —(C₃-C₇)-cycloalkyl, —C≡C-phenyl, —(C₃-C₇)-cycloalkyl, —C≡C—(C₁-C₅)-alkyl, and —O-phenyl, wherein phenyl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester, thioacyl, thioether, amino, amido, acylamino, cyano, and nitro;
each of R⁸ and R⁹ are independently selected from hydrogen, —(C₁-C₅)-alkyl, -aryl, -heteroaryl, —C(O)—(C₁-C₅)-alkyl, —C(O)-aryl, —C(O)-heteroaryl, —C(O)—O—(C₁-C₅)-alkyl, —C(O)—O-aryl, —C(O)—O-heteroaryl, and —C(O)—N(R^{a})(R^{b}), wherein any alkyl, aryl or heteroaryl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester, thioacyl, thioether, amino, amido, acylamino, cyano, and nitro;
each of R^{10a} and R^{10b} is independently selected from hydrogen, (C₁-C₅)-alkyl, perfluoroalkyl, O—(C₁-C₅)-alkyl, aryl and heteroaryl, or
R^{10a} and R^{10b} are taken together with the carbon atom to which they are bound to form a carbocyclic or heterocyclic ring; and
wherein each double bond is independently in an E- or a Z-configuration.

In certain embodiments, the present invention provides a pharmaceutical preparation suitable for use in a human patient, comprising an effective amount of any of the compounds shown above (e.g., a compound of the invention, such as a compound of any of formulae I-III), and one or more pharmaceutically acceptable excipients. In certain embodiments, the pharmaceutical preparations may be for use in treating or preventing a condition or disease as described herein. In certain embodiments, the pharmaceutical preparations have a low enough pyrogen activity to be suitable for use in a human patient.

The present invention further provides methods of treating or preventing bone metabolism, mucositis, cardiovascular disease, inflammatory diseases, metabolic diseases, ophthalmic conditions, immune function, pulmonary conditions, gastrointestinal conditions, rheumatological conditions, dermatological conditions, neurological conditions, cancer, infectious conditions, degenerative conditions, gerontological conditions, and apoptotic conditions, as described herein, comprising administering a compound of the invention.

### DETAILED DESCRIPTION OF THE INVENTION

The present invention provides a compound of formula I,

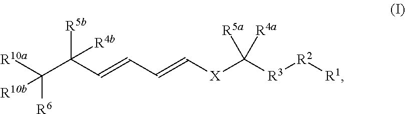

or a pharmaceutically acceptable salt thereof, wherein:
X is selected from —C≡C—, —C(R⁷)═C(R⁷)—, -(cyclopropyl)-, -(cyclobutyl)-, -(cyclopentyl)-, and -(cyclohexyl)-;
R¹ is selected from —OR^{a}, —N(R^{a})—SO₂—R^{c} and —N(R^{a})(R^{b}), wherein each of R^{a} and R^{b} is independently selected from H, C₁-C₆-alkyl, aryl, aralkyl, heteroaryl, and heteroaralkyl, and R^{c} is selected from C₁-C₆-alkyl, aryl, aralkyl, heteroaryl, and heteroaralkyl;
R² is selected from —CH₂—, —C(O)—, —SO₂—, —PO(OR)—, and tetrazole;
R is selected from hydrogen and alkyl;
R³ is selected from a carbocyclic ring, a heterocyclic ring, —(CH₂)ₙ—, CH₂C(O)CH₂, and —CH₂—O—CH₂, wherein:
   n is an integer from 1 to 3;
   any hydrogen atom in R³ is optionally and independently replaced by halo, (C₁-C₅)-alkyl, perfluoroalkyl, aryl, heteroaryl, hydroxy, or O—(C₁-C₅)-alkyl; and
   any two hydrogen atoms bound to a common carbon atom in R³ are optionally taken together with the carbon atom to which they are bound to form a carbocyclic or heterocyclic ring;
each of R^{4a} and R^{4b} is independently selected from hydrogen, halo, —OH, —O—(C₁-C₅)-alkyl, —O-aryl, O-heteroaryl, —O—C(O)—(C₁-C₅)-alkyl, —O—C(O)-aryl, —O—C(O)-heteroaryl, —O—C(O)—O—(C₁-C₅)-alkyl, —O—C(O)—O-aryl, —O—C(O)—O-heteroaryl, and —O—C(O)—N(R^{a})(R^{b}), wherein any alkyl, aryl or heteroaryl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester, thioacyl, thioether, amino, amido, acylamino, cyano, and nitro;
each of R^{5a} and R^{5b} is independently selected from hydrogen, halo, (C₁-C₅)-alkyl, perfluoroalkyl, aryl, and heteroaryl, preferably hydrogen, halo and (C₁-C₅)-alkyl;
R⁶ is selected from -phenyl, —(C₁-C₅)-alkyl, —(C₃-C₇)-cycloalkyl, —C≡C-phenyl, —C≡C—(C₃-C₇)-cycloalkyl, —C≡C—(C₁-C₅)-alkyl, and —O-phenyl, wherein phenyl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester, thioacyl, thioether, amino, amido, acylamino, cyano, and nitro,
   and R⁶ is additionally selected from —C≡CH when:
      a) X is —C(R⁷)═C(R⁷)— or -(cyclopropyl)-; or
      b) each of R^{4a} and R^{4b} is hydrogen or halo; or
      c) each of R^{5a} and R^{5b} is halo; or
      d) R² is —CH₂—;
each R⁷ is independently selected from hydrogen and (C₁-C₅)-alkyl, or two occurrences of R⁷ may optionally be taken together with the carbons to which they are attached to form a 5- or 6-membered ring;
each of R^{10a} and R^{10b} is independently selected from hydrogen, (C₁-C₅)-alkyl, perfluoroalkyl, O—(C₁-C₅)-alkyl, aryl and heteroaryl, or R^{10a} and R^{10b} are taken together with the carbon atom to which they are bound to form a carbocyclic or heterocyclic ring;
and each double bond is independently in an E- or a Z-configuration.

In certain embodiments, R¹ is —OM, where M is a cation selected from ammonium, tetra-alkyl ammonium, Na, K, Mg, and Zn.

In certain embodiments, R² and R¹ together are

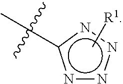

In certain embodiments, X is —C≡C—. In certain embodiments, X is —C(R⁷)═C(R⁷)—, -(cyclopropyl)-, -(cyclobutyl)-, -(cyclopentyl)-, or -(cyclohexyl)-. In certain embodiments, X is —C(R⁷)═C(R⁷)—. In certain embodiments, X is —C≡C—, -(cyclopropyl)-, -(cyclobutyl)-, -(cyclopentyl)-, or -(cyclohexyl)-. In certain embodiments, X is -(cyclopropyl)-. In certain embodiments, X is —C≡C— or —C(R⁷)═C(R⁷)—. In certain embodiments wherein X is -(cyclopropyl)-, -(cyclobutyl)-, -(cyclopentyl)-, or -(cyclohexyl)-, the olefin and the carbon bearing R^{4a} are attached to adjacent carbons on the -(cyclopropyl)-, -(cyclobutyl)-, -(cyclopentyl)-, or -(cyclohexyl)- ring system.

In certain embodiments, R^{4b} is hydrogen. In certain embodiments, R^{4b} is halo, —OH, —O—(C₁-C₅)-alkyl, —O-aryl, O-heteroaryl, —O—C(O)—(C₁-C₅)-alkyl, —O—C(O)-aryl, —O—C(O)-heteroaryl, —O—C(O)—O—(C₁-C₅)-alkyl, —O—C(O)—O-aryl, —O—C(O)—O-heteroaryl, or —O—C(O)—N(R^{a})(R^{b}), wherein any alkyl, aryl or heteroaryl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester, thioacyl, thioether, amino, amido, acylamino, cyano, and nitro. In certain embodiments, R^{4b} is fluoro. In certain embodiments, R^{4b} is hydrogen, —OH, —O—(C₁-C₅)-alkyl, —O-aryl, O-heteroaryl, —O—C(O)—(C₁-C₅)-alkyl, —O—C(O)-aryl, —O—C(O)-heteroaryl, —O—C(O)—O—(C₁-C₅)-alkyl, —O—C(O)—O-aryl, —O—C(O)—O-heteroaryl, or —O—C(O)—N(R^{a})(R^{b}), wherein any alkyl, aryl or heteroaryl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester, thioacyl, thioether, amino, amido, acylamino, cyano, and nitro. In certain embodiments, R^{4b} is selected from —OH, —O—(C₁-C₅)-alkyl, O-aryl, O-heteroaryl, —O—C(O)—(C₁-C₅)-alkyl, O—C(O)-aryl, O—C(O)-heteroaryl, and —O—C(O)—N(R^{a})(R^{b}). In certain embodiments, R^{4b} is hydrogen, halo, —O—C(O)—O—(C₁-C₅)-alkyl, —O—C(O)—O-aryl, or —O—C(O)—O-heteroaryl, wherein any alkyl, aryl or heteroaryl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester, thioacyl, thioether, amino, amido, acylamino, cyano, and nitro. In certain embodiments, R^{4b} is selected from hydrogen, halo, —OH, or —O—(C₁-C₅)-alkyl. In certain embodiments, R^{4b} is —O-aryl, O-heteroaryl, —O—C(O)—(C₁-C₅)-alkyl, —O—C(O)-aryl, —O—C(O)-heteroaryl, —O—C(O)—O—(C₁-C₅)-alkyl, —O—C(O)—O-aryl, —O—C(O)—O-heteroaryl, or —O—C(O)—N(R^{a})(R^{b}), wherein any alkyl, aryl or heteroaryl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester, thioacyl, thioether, amino, amido, acylamino, cyano, and nitro. In certain embodiments, R^{4b} is selected from —OH, —O—(C₁-C₅)-alkyl, —O-aryl, O-heteroaryl, —O—C(O)—(C₁-C₅)-alkyl, —O—C(O)-aryl, —O—C(O)-heteroaryl, —O—C(O)—O—(C₁-C₅)-alkyl, —O—C(O)—O-aryl, —O—C(O)—O-heteroaryl, and —O—C(O)—N(R^{a})(R^{b}), wherein any alkyl, aryl or heteroaryl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester, thioacyl, thioether, amino, amido, acylamino, cyano, and nitro. In certain embodiments, R^{4b} is selected from hydrogen or halo.

In certain embodiments, R^{4b} is in an (R) configuration. In certain embodiments, R^{4b} is in an (S) configuration.

In certain embodiments, R^{4a} is hydrogen. In certain embodiments, R^{4a} is halo, —OH, —O—(C₁-C₅)-alkyl, —O-aryl, O-heteroaryl, —O—C(O)—(C₁-C₅)-alkyl, —O—C(O)-aryl, —O—C(O)-heteroaryl, —O—C(O)—O—(C₁-C₅)-alkyl, —O—C(O)—O-aryl, —O—C(O)—O-heteroaryl, or —O—C(O)—N(R^{a})(R^{b}), wherein any alkyl, aryl or heteroaryl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester, thioacyl, thioether, amino, amido, acylamino, cyano, and nitro. In certain embodiments, R^{4a} is fluoro. In certain embodiments, R^{4a} is hydrogen, —OH, —O—(C₁-C₅)-alkyl, —O-aryl, O-heteroaryl, —O—C(O)—(C₁-C₅)-alkyl, —O—C(O)-aryl, —O—C(O)-heteroaryl, —O—C(O)—O—(C₁-C₅)-alkyl, —O—C(O)—O-aryl, —O—C(O)—O-heteroaryl, or —O—C(O)—N(R^{a})(R^{b}), wherein any alkyl, aryl or heteroaryl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester, thioacyl, thioether, amino, amido, acylamino, cyano, and nitro. In certain embodiments, R^{4a} is selected from —OH, —O—(C₁-C₅)-alkyl, O-aryl, O-heteroaryl, —O—C(O)—(C₁-C₅)-alkyl, O—C(O)-aryl, O—C(O)-heteroaryl, and —O—C(O)—N(R^{a})(R^{b}). In certain embodiments, R^{4a} is hydrogen, halo, —O—C(O)—O—(C₁-C₅)-alkyl, —O—C(O)—O-aryl, or —O—C(O)—O-heteroaryl, wherein any alkyl, aryl or heteroaryl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester, thioacyl, thioether, amino, amido, acylamino, cyano, and nitro. In certain embodiments, R^{4a} is selected from hydrogen, halo, —OH, or —O—(C₁-C₅)-alkyl. In certain embodiments, R^{4a} is —O-aryl, O-heteroaryl, —O—C(O)—(C₁-C₅)-alkyl, —O—C(O)-aryl, —O—C(O)-heteroaryl, —O—C(O)—O—(C₁-C₅)-alkyl, —O—C(O)—O-aryl, —O—C(O)—O-heteroaryl, or —O—C(O)—N(R^{a})(R^{b}), wherein any alkyl, aryl or heteroaryl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester, thioacyl, thioether, amino, amido, acylamino, cyano, and nitro. In certain embodiments, R^{4a} is selected from —OH, —O—(C₁-C₅)-alkyl, —O-aryl, O-heteroaryl, —O—C(O)—(C₁-C₅)-alkyl, —O—C(O)-aryl, —O—C(O)-heteroaryl, —O—C(O)—O—(C₁-C₅)-alkyl, —O—C(O)—O-aryl, —O—C(O)—O-heteroaryl, and —O—C(O)—N(R^{a})(R^{b}), wherein any alkyl, aryl or heteroaryl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester, thioacyl, thioether, amino, amido, acylamino, cyano, and nitro. In certain embodiments, R^{4a} is selected from hydrogen or halo.

In certain embodiments, R^{4a} is in an (S) configuration. In certain embodiments, R^{4a} is in an (R) configuration.

In certain embodiments wherein R^{4a} is —OH, R^{5a} is selected from hydrogen or (C₁-C₅)-alkyl. In certain embodiments wherein R^{4a} is selected from —OH, —O—(C₁-C₅)-alkyl, —O-aryl, O-heteroaryl, —O—C(O)—(C₁-C₅)-alkyl, —O—C(O)-aryl, —O—C(O)-heteroaryl, —O—C(O)—O—(C₁-C₅)-alkyl, —O—C(O)—O-aryl, —O—C(O)—O-heteroaryl, and —O—C(O)—N(R^{a})(R^{b}), R^{5a} is selected from hydrogen or (C₁-C₅)-alkyl. In certain embodiments, R^{5a} is fluoro. In certain embodiments, R^{5a} is selected from hydrogen and (C₁-C₅)-alkyl.

In certain embodiments wherein R^{4b} is —OH, R^{5b} is selected from hydrogen or (C₁-C₅)-alkyl. In certain embodiments wherein R^{4b} is selected from —OH, —O—(C₁-C₅)-alkyl, —O-aryl, O-heteroaryl, —O—C(O)—(C₁-C₅)-alkyl, —O—C(O)-aryl, —O—C(O)-heteroaryl, —O—C(O)—O—(C₁-C₅)-alkyl, —O—C(O)—O-aryl, —O—C(O)—O-heteroaryl, and —O—C(O)—N(R^{a})(R^{b}), R^{5b} is selected from hydrogen or (C₁-C₅)-alkyl. In certain embodiments, R^{5b} is fluoro. In certain embodiments, R^{5b} is selected from hydrogen and (C₁-C₅)-alkyl.

In certain embodiments, R² is —CH₂—. In certain embodiments, R² is —C(O)—.

In certain embodiments, R^{a} is selected from H and C₁-C₆-alkyl. In certain embodiments, R^{a} is selected from aryl, aralkyl, heteroaryl, and heteroaralkyl.

In certain embodiments, R^{b} is selected from H and C₁-C₆-alkyl. In certain embodiments, R^{b} is selected from aryl, aralkyl, heteroaryl, and heteroaralkyl.

In certain embodiments, R^{c} is C₁-C₆-alkyl, aryl, or heteroaryl. In certain embodiments, R^{c} is selected from aryl, aralkyl, heteroaryl, and heteroaralkyl.

In certain embodiments wherein R³ is selected from a carbocyclic ring, a heterocyclic ring, —(CH₂)ₙ—, and CH₂C(O)CH₂, any hydrogen atom in R³ is optionally and independently replaced by halo, (C₁-C₅)-alkyl, perfluoroalkyl, aryl, heteroaryl, hydroxy, or O—(C₁-C₅)-alkyl. In certain embodiments wherein R³ is —CH₂—O—CH₂, any hydrogen atom in R³ is optionally and independently replaced by halo, (C₁-C₅)-alkyl, perfluoroalkyl, aryl, heteroaryl, or O—(C₁-C₅)-alkyl. In certain embodiments, R³ is selected from —(CH₂)ₙ— and —CH₂—O—CH₂, wherein n is an integer from 1 to 3, and up to two hydrogen atoms in R³ are optionally and independently replaced by (C₁-C₅)-alkyl. In certain embodiments, R³ is selected from a carbocyclic ring, a heterocyclic ring, and CH₂C(O)CH₂, wherein n is an integer from 1 to 3; any hydrogen atom in R³ is optionally and independently replaced by halo, (C₁-C₅)-alkyl, perfluoroalkyl, aryl, heteroaryl, hydroxy, or O—(C₁-C₅)-alkyl; and any two hydrogen atoms bound to a common carbon atom in R³ are optionally taken together with the carbon atom to which they are bound to form a carbocyclic or heterocyclic ring.

In certain embodiments, R^{10a} is hydrogen. In certain embodiments, R^{10a} is selected from (C₁-C₅)-alkyl, perfluoroalkyl, O—(C₁-C₅)-alkyl, aryl and heteroaryl, or R^{10a} is taken together with R^{10b} and the carbon atom to which they are bound to form a carbocyclic or heterocyclic ring.

In certain embodiments, R^{10b} is hydrogen. In certain embodiments, R^{10b} is selected from (C₁-C₅)-alkyl, perfluoroalkyl, O—(C₁-C₅)-alkyl, aryl and heteroaryl, or R^{10b} is taken together with R^{10a} and the carbon atom to which they are bound to form a carbocyclic or heterocyclic ring.

In certain embodiments, R¹ is —OR^{a}. In certain embodiments, R¹ is selected from —N(R^{a})—SO₂—R^{c} and —N(R^{a})(R^{b}). In certain embodiments, R¹ is —N(R^{a})—SO₂—R^{c}. In certain embodiments, R¹ is selected from —OR^{a} and —N(R^{a})(R^{b}). In certain embodiments, R¹ is —N(R^{a})(R^{b}). In certain embodiments, R¹ is selected from —OR^{a}, and —N(R^{a})—SO₂—R^{c}.

In certain embodiments, R⁷ is hydrogen. In certain embodiments, R⁷ is (C₁-C₅)-alkyl or two occurrences of R⁷ may optionally be taken together with the carbons to which they are attached to form a 5- or 6-membered ring.

In certain embodiments, X is —C≡C— and R^{4b} is hydrogen.

In certain embodiments, X is —C≡C— and R^{4a} is hydrogen.

In certain embodiments, X is —C≡C—, R^{4a} is fluoro, and R^{5a} is fluoro.

In certain embodiments, X is —C≡C—, R^{4b} is fluoro, and R^{5b} is fluoro.

In certain embodiments, X is —C≡C—, and each of R^{4a} and R^{4b} is independently selected from —OH, —O—(C₁-C₅)-alkyl, O-aryl, O-heteroaryl, —O—C(O)—(C₁-C₅)-alkyl, O—C(O)-aryl, O—C(O)-heteroaryl, and —O—C(O)—N(R^{a})(R^{b}).

In certain embodiments, X is —C≡C— and R² is —CH₂—.

In certain embodiments, X is -(cyclopropyl)-, -(cyclobutyl)-, -(cyclopentyl)-, and -(cyclohexyl)-. In certain embodiments, X is -(cyclopropyl)-.

In certain embodiments, X is —C(R⁷)═C(R⁷)—.

In certain embodiments, each of R^{a} and R^{b} is independently selected from H and C₁-C₆-alkyl; R^{c} is C₁-C₆-alkyl; R³ is selected from —(CH₂)ₙ— and —CH₂—O—CH₂, wherein n is an integer from 1 to 3, and up to two hydrogen atoms in R³ are optionally and independently replaced by (C₁-C₅)-alkyl; each of R^{4a} and R^{4b} is independently selected from hydrogen, halo, —OH, —O—(C₁-C₅)-alkyl; and each of R^{10a} and R^{10b} is hydrogen.

In certain embodiments, each double bond is in an E-configuration. In certain embodiments, each double bond is in a Z-configuration. In certain embodiments, one double bond is in an E-configuration and one double bond is in a Z-configuration.

In certain embodiments, the invention contemplates any combination of the foregoing. Those skilled in the art will recognize that all specific combinations of the individual possible residues of the variable regions of the compounds as disclosed R¹, R², R³, R^{4a}, R^{4b}, R^{5a}, R^{5b}, R⁶, R⁷, R^{10a}, R^{10b}, R^{a}, R^{b}, R^{c}, n and X, are within the scope of the invention. As an example, any of the various particular recited embodiments for R^{4a} may be combined with any of the various particular recited embodiments of X.

In certain embodiments, the compound is selected from any one of:

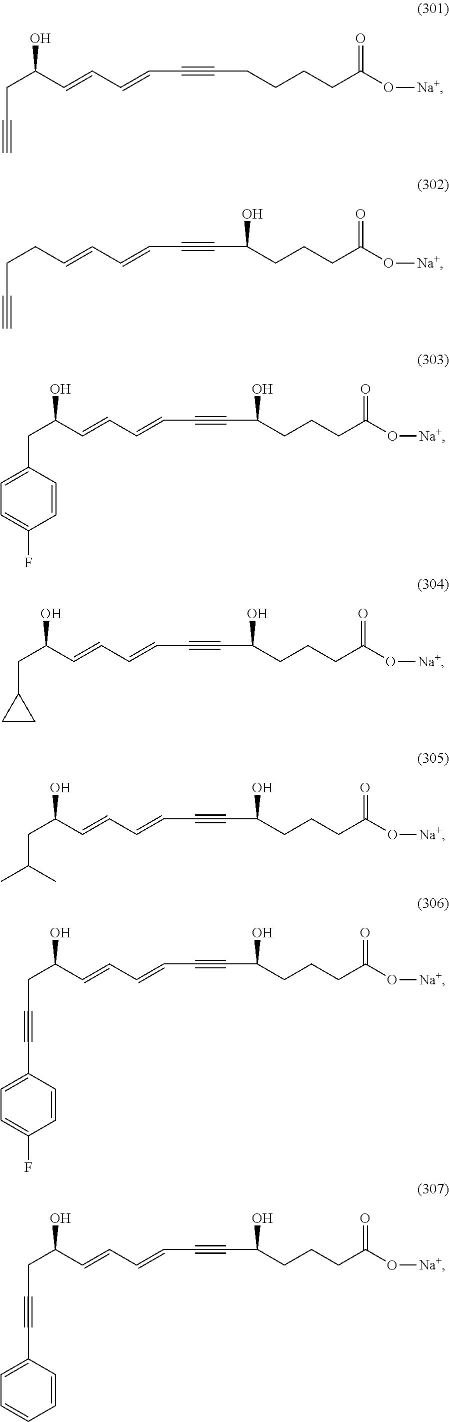

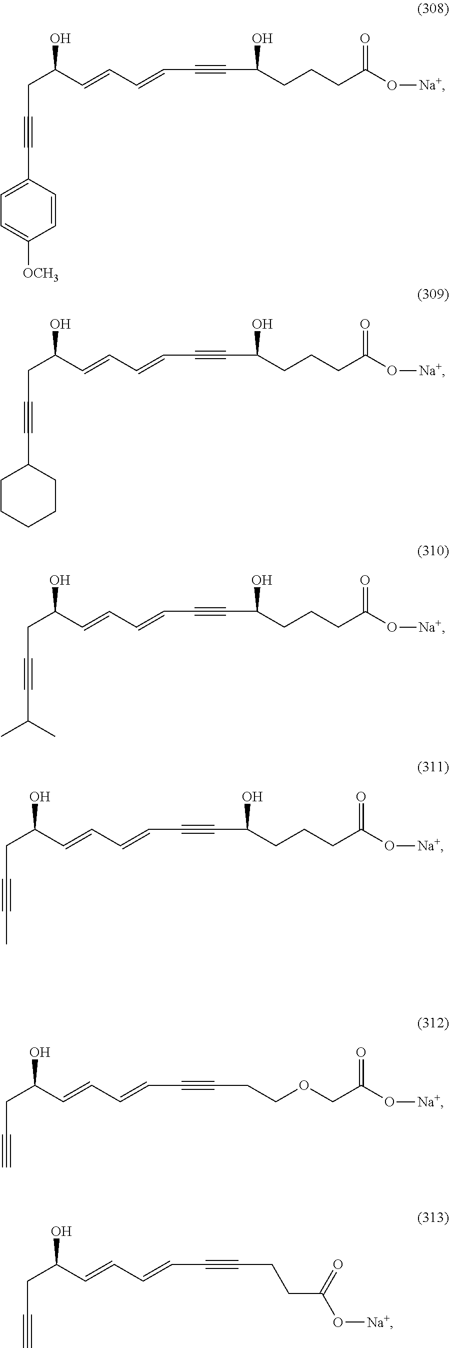

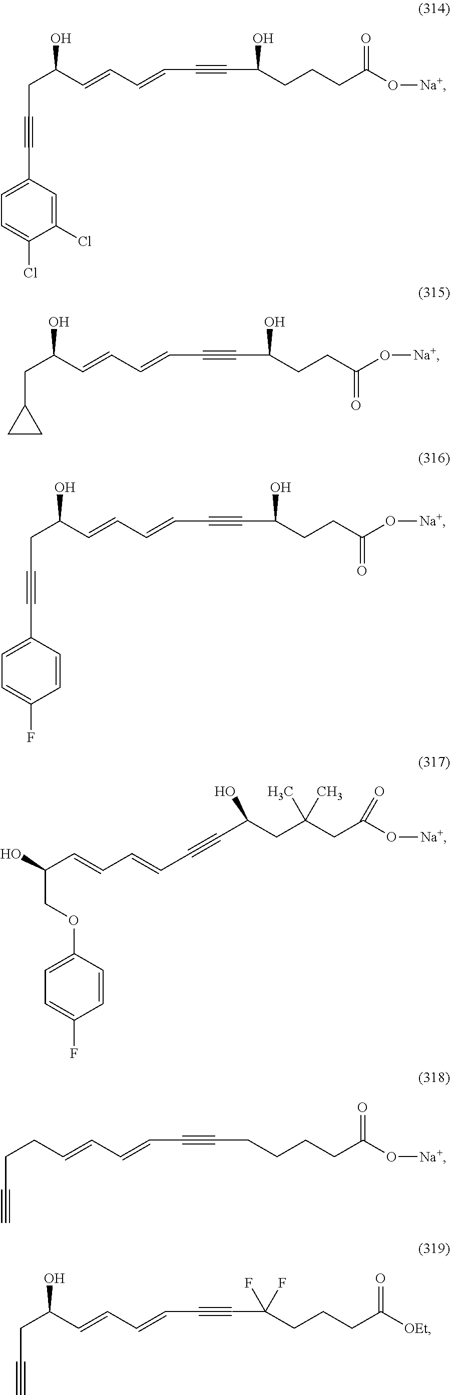

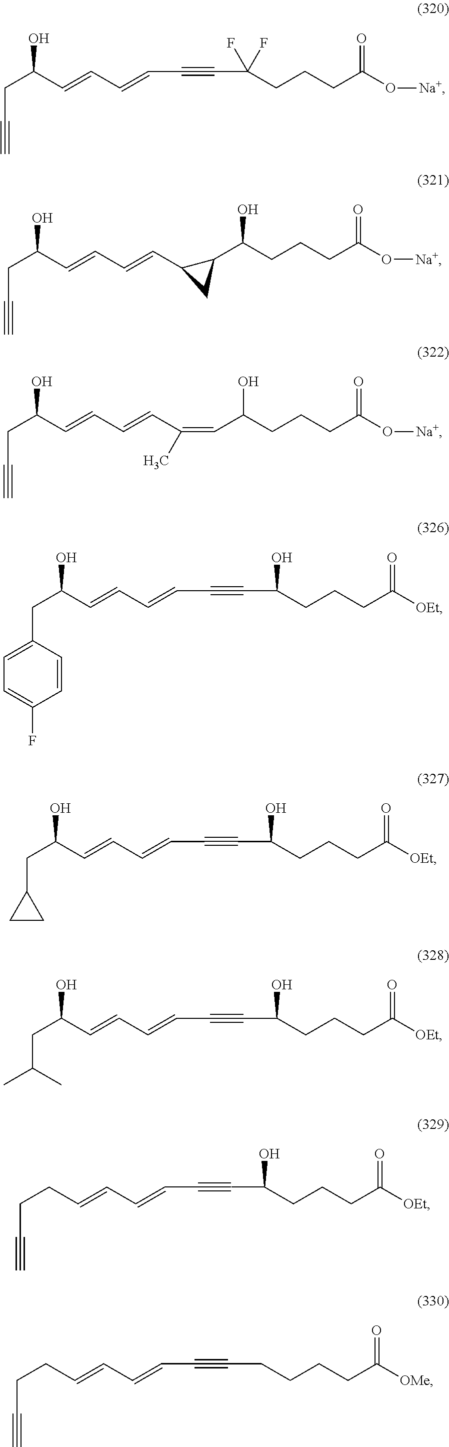

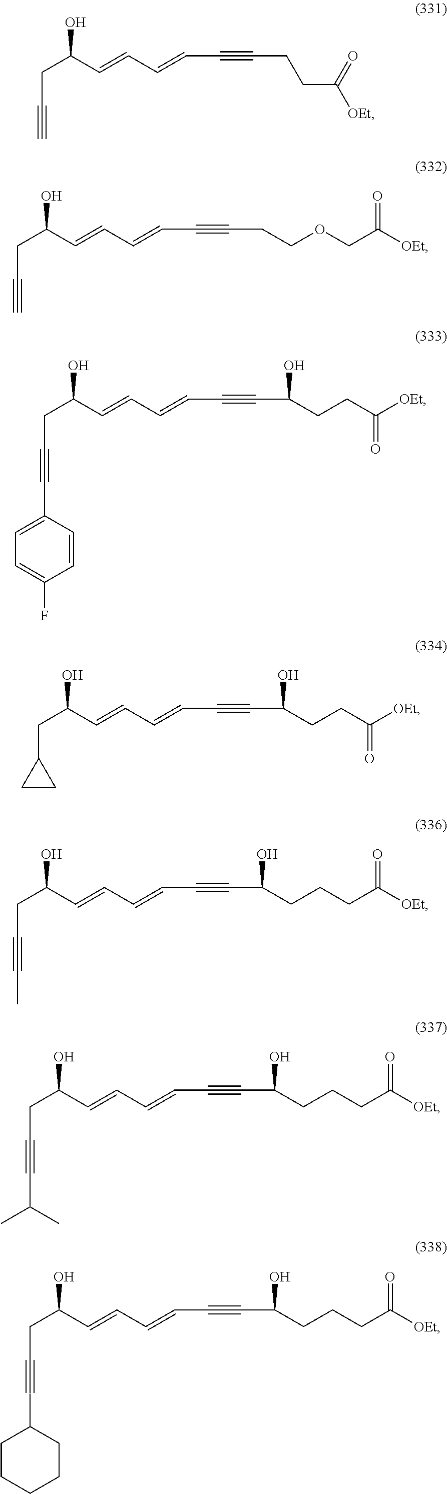

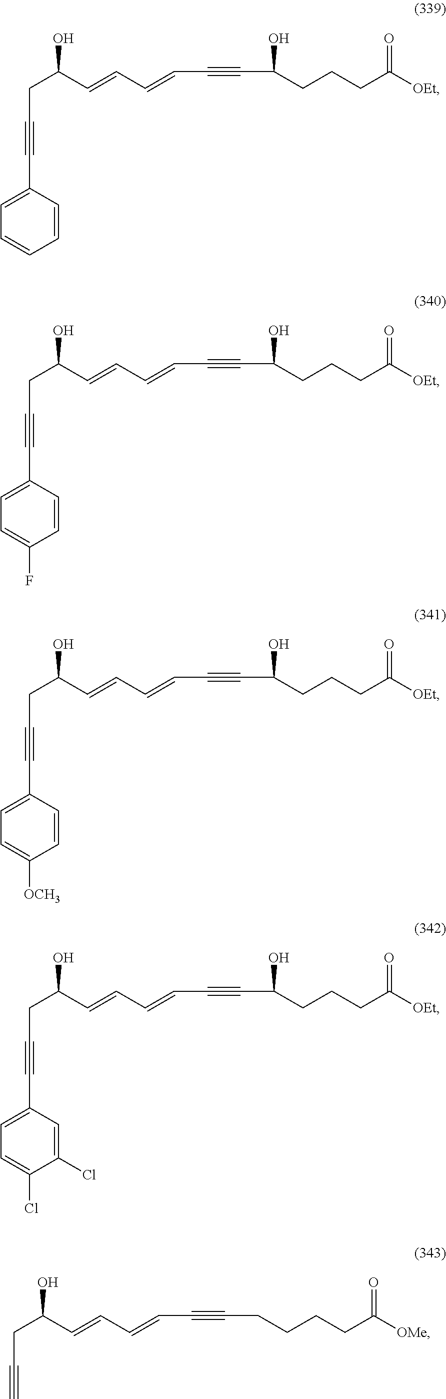

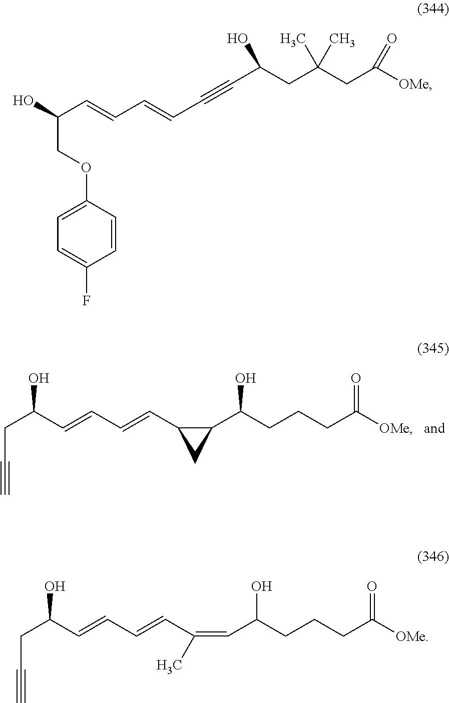

The present invention provides a compound of the formula II,

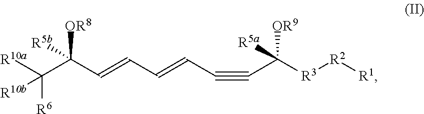

or formula III,

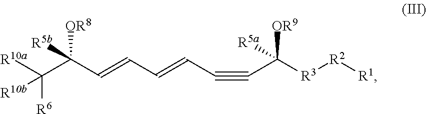

or a pharmaceutically
acceptable
salt of either of the foregoing, wherein:
R¹ is selected from —OR^{a}, —N(R^{a})—SO₂—R^{c} and —N(R^{a})(R^{b}), wherein each of R^{a} and R^{b} is independently selected from H, C₁-C₆-alkyl, aryl, aralkyl, heteroaryl, and heteroaralkyl, and R^{c} is selected from C₁-C₆-alkyl, aryl, aralkyl, heteroaryl, and heteroaralkyl;
R² is selected from —C(O)—, —SO₂—, —PO(OR)—, and tetrazole;
R is selected from hydrogen and alkyl;
R³ is selected from —(CH₂)ₙ— and —CH₂—O—CH₂, wherein n is an integer from 1 to 3; and optionally up to two hydrogen atoms in R³ are independently replaced by halo, (C₁-C₅)-alkyl, or O—(C₁-C₅)-alkyl;
each of R^{5a} and R^{5b} is independently selected from hydrogen, (C₁-C₅)-alkyl, perfluoroalkyl, aryl, and heteroaryl, preferably hydrogen and (C₁-C₅)-alkyl;
R⁶ is selected from —C≡CH, -phenyl, —(C₁-C₅)-alkyl, —(C₃-C₇)-cycloalkyl, —C≡C-phenyl, —C≡C—(C₃-C₇)-cycloalkyl, —C≡C—(C₁-C₅)-alkyl, and —O-phenyl, wherein phenyl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester, thioacyl, thioether, amino, amido, acylamino, cyano, and nitro;
each of R⁸ and R⁹ are independently selected from hydrogen, —(C₁-C₅)-alkyl, -aryl, -heteroaryl, —C(O)—(C₁-C₅)-alkyl, —C(O)-aryl, —C(O)-heteroaryl, —C(O)—O—(C₁-C₅)-alkyl, —C(O)—O-aryl, —C(O)—O-heteroaryl, and —C(O)—N(R^{a})(R^{b}), wherein any alkyl, aryl or heteroaryl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester, thioacyl, thioether, amino, amido, acylamino, cyano, and nitro;
each of R^{10a} and R^{10b} is independently selected from hydrogen, (C₁-C₅)-alkyl, perfluoroalkyl, O—(C₁-C₅)-alkyl, aryl and heteroaryl, or
R^{10a} and R^{10b} are taken together with the carbon atom to which they are bound to form a carbocyclic or heterocyclic ring; and
wherein each double bond is independently in an E- or a Z-configuration.

In certain embodiments, R¹ is —OM, where M is a cation selected from ammonium, tetra-alkyl ammonium, Na, K, Mg, and Zn.

In certain embodiments, R² and R¹ together are

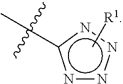

In certain embodiments, R² is —C(O)—. In certain embodiments, R¹ is —OR^{a}, wherein R^{a} is hydrogen or C₁-C₆-alkyl. In certain embodiments, R³ is —(CH₂)ₙ—, wherein n is 3. In certain embodiments, R⁶ is —C≡CH. In certain embodiments, R^{5a} is hydrogen. In certain embodiments, R^{5b} is hydrogen. In certain embodiments, R^{10a} is hydrogen. In certain embodiments, R^{10b} is hydrogen. In certain embodiments, R² is —C(O)—, R¹ is —OR^{a}, wherein R^{a} is C₁-C₆-alkyl, R³ is —(CH₂)ₙ—, wherein n is 3, R⁶ is —C≡CH, R^{5a} is hydrogen, R^{5b} is hydrogen, R^{10a} is hydrogen, and R^{10b} is hydrogen.

In certain embodiments, the compound is selected from any one of:

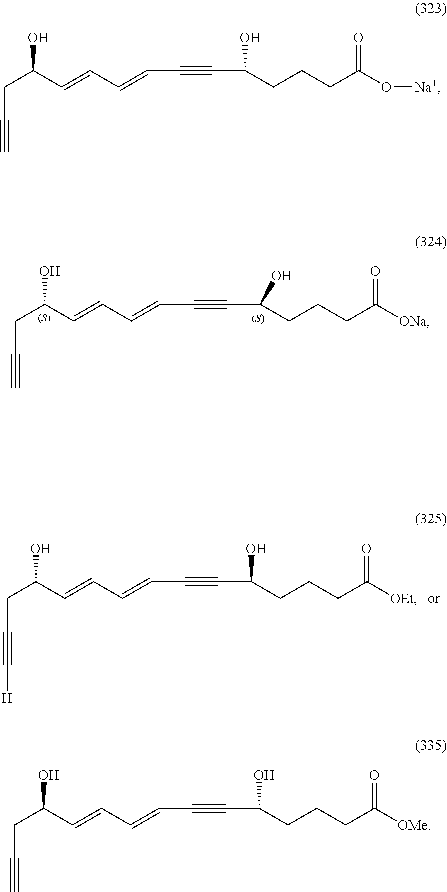

In certain embodiments, the invention contemplates any combination of the foregoing. Those skilled in the art will recognize that all specific combinations of the individual possible residues of the variable regions of the compounds as disclosed herein, e.g., R¹, R², R³, R^{5a}, R^{5b}, R⁶, R⁸, R⁹, R^{10a}, R^{10b}, R^{a}, R^{b}, R^{c}, and n, are within the scope of the invention. As an example, any of the various particular recited embodiments for R⁸ may be combined with any of the various particular recited embodiments of R⁶.

In certain embodiments, the present invention provides a pharmaceutical preparation suitable for use in a human patient, comprising an effective amount of any of the compounds shown above (e.g., a compound of the invention, such as a compound of any of formulae I-III), and one or more pharmaceutically acceptable excipients. In certain embodiments, the pharmaceutical preparations may be for use in treating or preventing a condition or disease as described herein. In certain embodiments, the pharmaceutical preparations have a low enough pyrogen activity to be suitable for use in a human patient.

Compounds of any of the above structures may be used in the manufacture of medicaments for the treatment of any diseases or conditions disclosed herein.

Bone Metabolism

The present invention provides methods of treating or preventing bone loss in a patient comprising administering a compound of the invention. In certain embodiments, conditions with which the bone loss is associated include, for example, but are not limited to any one or more of: ankylosing spondylitis, renal osteodystrophy (e.g., in patients undergoing dialysis), osteoporosis, glucocorticoid-induced osteoporosis, Paget's disease, abnormally increased bone turnover, periodontitis, periodontal disease, bone fractures, rheumatoid arthritis, osteoarthritis, periprosthetic osteolysis, osteogenesis imperfecta, metastatic bone disease, hypercalcemia of malignancy, multiple myeloma, bone loss associated with microgravity, Langerhan's Cell Histiocytosis (LHC), bone loss associated with renal tubular disorders, or bone loss associated with bed-ridden conditions.

The present invention provides a method of treating or preventing osteoporosis in a patient comprising administering a compound of the invention. In certain embodiments, the osteoporosis is medicine-induced osteoporosis. In certain embodiments, the medicine-induced osteoporosis is glucocorticoid-induced osteoporosis.

The present invention provides a method of treating or preventing diabetic osteopenia in a patient comprising administering a compound of the invention.

The present invention provides a method of treating or preventing metastatic bone disease in a patient comprising administering a compound of the invention. The present invention provides a method of decreasing the incidence of bone metastasis in a patient comprising administering a compound of the invention. The present invention further provides a method of delaying the onset of bone metastasis in a patient comprising administering a compound of the invention. In certain embodiments, a compound of the invention is administered conjointly with chemotherapy or radiation therapy.

The present invention provides a method of treating or preventing periodontitis in a patient comprising administering a compound of the invention.

The present invention provides a method of treating or preventing gingivitis in a patient comprising administering a compound of the invention.

The present invention provides a method of treating or preventing ankylosing spondylitis in a patient comprising administering a compound of the invention.

The present invention provides a method of treating or preventing renal osteodystrophy (e.g., in patients undergoing dialysis) in a patient comprising administering a compound of the invention.

In one embodiment, the method of treating or preventing bone loss may comprise administering a compound of the invention conjointly with an additional agent useful in the treatment of bone loss. In certain embodiments, the compound of the invention may be conjointly administered with a bisphosphonate (e.g., ibandronate, zolendronate, risedronate, etidronate, or alendronate), a steroid, such as an anabolic steroid (e.g., testosterone, quinbolone, oxymetholone, nandrolone hexylphenylpropionate, oxandrolone, testosterone undecanoate, mibolerone, danozol, nandrolone decanoate, trenbolone cyclohexylmethylcarbonate, methenolone acetate, methenolone enanthate, mesterolone, dihydrotestosterone, methandrostenolone, nandrolone undecanoate, boldenone undecylenate, formebolone, trenbolone acetate, fluoxymesterone, nandrolone laureate, drostanolone propionate, clostebol acetate, trestolone acetate, methandriol dipropionate, methyltestosterone, furazabol, bolasterone, norethandrolone, mepitiostane, tetrahydrogestrinone, trenbolone enanthate, and stanozolol), an estrogen (e.g., estradiol, estriol, estrone, equilin, or equilenin), a substance having estrogenic activity (e.g., xenoestrogens, phytoestrogens, or mycoestrogens), a selective estrogen receptor modulator (e.g., raloxifene), or hormone treatment (e.g., calcitonin or teriparitide). In certain embodiments, the compound of the invention may be conjointly administered with growth factors or other therapeutic agents that have a positive effect on the growth of bone or connective tissue, such as osteoprotegerin, interleukins, MMP inhibitors, beta glucans, integrin antagonists, calcitonin, proton pump inhibitors, protease inhibitors, insulin-like growth factor-1, platelet-derived growth factor, epidermal growth factor, inhibitors of transforming growth factor-alpha, transforming growth factor-beta, bone morphogenetic protein, parathyroid hormone, osteoprotegerin, a fibroblast growth factor, Vitamin D, vitronectin, plasminogen-activator inhibitor, or a protease inhibitor such as a metalloprotease inhibitor, or elements known to be beneficial to bone formation, such as calcium, fluoride, magnesium, boron, or a combination thereof.

In one embodiment, the method of treating or preventing metastatic bone disease may comprise administering a compound of the invention conjointly with a chemotherapeutic agent. Chemotherapeutic agents that may be conjointly administered with compounds of the invention include: aminoglutethimide, amsacrine, anastrozole, asparaginase, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

Many combination therapies have been developed for the treatment of cancer. In certain embodiments, compounds of the invention may be conjointly administered with a combination therapy. Examples of combination therapies with which compounds of the invention may be conjointly administered are included in Table 1.

TABLE 1
Exemplary combinatorial therapies for the treatment of cancer.

| Name | Therapeutic agents |
|---|---|
| ABV | Doxorubicin, Bleomycin, Vinblastine |
| ABVD | Doxorubicin, Bleomycin, Vinblastine, Dacarbazine |
| AC (Breast) | Doxorubicin, Cyclophosphamide |
| AC (Sarcoma) | Doxorubicin, Cisplatin |
| AC (Neuroblastoma) | Cyclophosphamide, Doxorubicin |
| ACE | Cyclophosphamide, Doxorubicin, Etoposide |
| ACe | Cyclophosphamide, Doxorubicin |
| AD | Doxorubicin, Dacarbazine |
| AP | Doxorubicin, Cisplatin |
| ARAC-DNR | Cytarabine, Daunorubicin |
| B-CAVe | Bleomycin, Lomustine, Doxorubicin, Vinblastine |
| BCVPP | Carmustine, Cyclophosphamide, Vinblastine, |
| | Procarbazine, Prednisone |
| BEACOPP | Bleomycin, Etoposide, Doxorubicin, Cyclophosphamide, Vincristine, |
| | Procarbazine, Prednisone, Filgrastim |
| BEP | Bleomycin, Etoposide, Cisplatin |
| BIP | Bleomycin, Cisplatin, Ifosfamide, Mesna |
| BOMP | Bleomycin, Vincristine, Cisplatin, Mitomycin |
| CA | Cytarabine, Asparaginase |
| CABO | Cisplatin, Methotrexate, Bleomycin, Vincristine |
| CAF | Cyclophosphamide, Doxorubicin, Fluorouracil |
| CAL-G | Cyclophosphamide, Daunorubicin, Vincristine, |
| | Prednisone, Asparaginase |
| CAMP | Cyclophosphamide, Doxorubicin, Methotrexate, |
| | Procarbazine |
| CAP | Cyclophosphamide, Doxorubicin, Cisplatin |
| CaT | Carboplatin, Paclitaxel |
| CAV | Cyclophosphamide, Doxorubicin, Vincristine |
| CAVE ADD | CAV and Etoposide |
| CA-VP16 | Cyclophosphamide, Doxorubicin, Etoposide |
| CC | Cyclophosphamide, Carboplatin |
| CDDP/VP-16 | Cisplatin, Etoposide |
| CEF | Cyclophosphamide, Epirubicin, Fluorouracil |
| CEPP(B) | Cyclophosphamide, Etoposide, Prednisone, with or |
| | without/Bleomycin |
| CEV | Cyclophosphamide, Etoposide, Vincristine |
| CF | Cisplatin, Fluorouracil or Carboplatin Fluorouracil |
| CHAP | Cyclophosphamide or Cyclophosphamide, Altretamine, |
| | Doxorubicin, Cisplatin |
| ChlVPP | Chlorambucil, Vinblastine, Procarbazine, Prednisone |
| CHOP | Cyclophosphamide, Doxorubicin, Vincristine, Prednisone |
| CHOP-BLEO | Add Bleomycin to CHOP |
| CISCA | Cyclophosphamide, Doxorubicin, Cisplatin |
| CLD-BOMP | Bleomycin, Cisplatin, Vincristine, Mitomycin |
| CMF | Methotrexate, Fluorouracil, Cyclophosphamide |
| CMFP | Cyclophosphamide, Methotrexate, Fluorouracil, |
| | Prednisone |
| CMFVP | Cyclophosphamide, Methotrexate, Fluorouracil, |
| | Vincristine, Prednisone |
| CMV | Cisplatin, Methotrexate, Vinblastine |
| CNF | Cyclophosphamide, Mitoxantrone, Fluorouracil |
| CNOP | Cyclophosphamide, Mitoxantrone, Vincristine, Prednisone |
| COB | Cisplatin, Vincristine, Bleomycin |
| CODE | Cisplatin, Vincristine, Doxorubicin, Etoposide |
| COMLA | Cyclophosphamide, Vincristine, Methotrexate, |
| | Leucovorin, Cytarabine |
| COMP | Cyclophosphamide, Vincristine, Methotrexate, Prednisone |
| Cooper Regimen | Cyclophosphamide, Methotrexate, Fluorouracil, |
| | Vincristine, Prednisone |
| COP | Cyclophosphamide, Vincristine, Prednisone |
| COPE | Cyclophosphamide, Vincristine, Cisplatin, Etoposide |
| COPP | Cyclophosphamide, Vincristine, Procarbazine, Prednisone |
| CP(Chronic | Chlorambucil, Prednisone |
| lymphocytic leukemia) | |
| CP (Ovarian Cancer) | Cyclophosphamide, Cisplatin |
| CT | Cisplatin, Paclitaxel |
| CVD | Cisplatin, Vinblastine, Dacarbazine |
| CVI | Carboplatin, Etoposide, Ifosfamide, Mesna |
| CVP | Cyclophosphamide, Vincristine, Prednisome |
| CVPP | Lomustine, Procarbazine, Prednisone |
| CYVADIC | Cyclophosphamide, Vincristine, Doxorubicin, |
| | Dacarbazine |
| DA | Daunorubicin, Cytarabine |
| DAT | Daunorubicin, Cytarabine, Thioguanine |
| DAV | Daunorubicin, Cytarabine, Etoposide |
| DCT | Daunorubicin, Cytarabine, Thioguanine |
| DHAP | Cisplatin, Cytarabine, Dexamethasone |
| DI | Doxorubicin, Ifosfamide |
| DTIC/Tamoxifen | Dacarbazine, Tamoxifen |
| DVP | Daunorubicin, Vincristine, Prednisone |
| EAP | Etoposide, Doxorubicin, Cisplatin |
| EC | Etoposide, Carboplatin |
| EFP | Etoposie, Fluorouracil, Cisplatin |
| ELF | Etoposide, Leucovorin, Fluorouracil |
| EMA 86 | Mitoxantrone, Etoposide, Cytarabine |
| EP | Etoposide, Cisplatin |
| EVA | Etoposide, Vinblastine |
| FAC | Fluorouracil, Doxorubicin, Cyclophosphamide |
| FAM | Fluorouracil, Doxorubicin, Mitomycin |
| FAMTX | Methotrexate, Leucovorin, Doxorubicin |
| FAP | Fluorouracil, Doxorubicin, Cisplatin |
| F-CL | Fluorouracil, Leucovorin |
| FEC | Fluorouracil, Cyclophosphamide, Epirubicin |
| FED | Fluorouracil, Etoposide, Cisplatin |
| FL | Flutamide, Leuprolide |
| FZ | Flutamide, Goserelin acetate implant |
| HDMTX | Methotrexate, Leucovorin |
| Hexa-CAF | Altretamine, Cyclophosphamide, Methotrexate, |
| | Fluorouracil |
| ICE-T | Ifosfamide, Carboplatin, Etoposide, Paclitaxel, Mesna |
| IDMTX/6-MP | Methotrexate, Mercaptopurine, Leucovorin |
| IE | Ifosfamide, Etoposie, Mesna |
| IfoVP | Ifosfamide, Etoposide, Mesna |
| IPA | Ifosfamide, Cisplatin, Doxorubicin |
| M-2 | Vincristine, Carmustine, Cyclophosphamide, Prednisone, |
| | Melphalan |
| MAC-III | Methotrexate, Leucovorin, Dactinomycin, |
| | Cyclophosphamide |
| MACC | Methotrexate, Doxorubicin, Cyclophosphamide, |
| | Lomustine |
| MACOP-B | Methotrexate, Leucovorin, Doxorubicin, |
| | Cyclophosphamide, Vincristine, Bleomycin, Prednisone |
| MAID | Mesna, Doxorubicin, Ifosfamide, Dacarbazine |
| m-BACOD | Bleomycin, Doxorubicin, Cyclophosphamide, Vincristine, |
| | Dexamethasone, Methotrexate, Leucovorin |
| MBC | Methotrexate, Bleomycin, Cisplatin |
| MC | Mitoxantrone, Cytarabine |
| MF | Methotrexate, Fluorouracil, Leucovorin |
| MICE | Ifosfamide, Carboplatin, Etoposide, Mesna |
| MINE | Mesna, Ifosfamide, Mitoxantrone, Etoposide |
| mini-BEAM | Carmustine, Etoposide, Cytarabine, Melphalan |
| MOBP | Bleomycin, Vincristine, Cisplatin, Mitomycin |
| MOP | Mechlorethamine, Vincristine, Procarbazine |
| MOPP | Mechlorethamine, Vincristine, Procarbazine, Prednisone |
| MOPP/ABV | Mechlorethamine, Vincristine, Procarbazine, Prednisone, |
| | Doxorubicin, Bleomycin, Vinblastine |
| MP (multiple | Melphalan, Prednisone |
| myeloma) | |
| MP (prostate cancer) | Mitoxantrone, Prednisone |
| MTX/6-MO | Methotrexate, Mercaptopurine |
| MTX/6-MP/VP | Methotrexate, Mercaptopurine, Vincristine, Prednisone |
| MTX-CDDPAdr | Methotrexate, Leucovorin, Cisplatin, Doxorubicin |
| MV (breast cancer) | Mitomycin, Vinblastine |
| MV (acute myelocytic | Mitoxantrone, Etoposide |
| leukemia) | |
| M-VAC Methotrexate | Vinblastine, Doxorubicin, Cisplatin |
| MVP Mitomycin | Vinblastine, Cisplatin |
| MVPP | Mechlorethamine, Vinblastine, Procarbazine, Prednisone |
| NFL | Mitoxantrone, Fluorouracil, Leucovorin |
| NOVP | Mitoxantrone, Vinblastine, Vincristine |
| OPA | Vincristine, Prednisone, Doxorubicin |
| OPPA | Add Procarbazine to OPA. |
| PAC | Cisplatin, Doxorubicin |
| PAC-I | Cisplatin, Doxorubicin, Cyclophosphamide |
| PA-CI | Cisplatin, Doxorubicin |
| PC | Paclitaxel, Carboplatin or Paclitaxel, Cisplatin |
| PCV | Lomustine, Procarbazine, Vincristine |
| PE | Paclitaxel, Estramustine |
| PFL | Cisplatin, Fluorouracil, Leucovorin |
| POC | Prednisone, Vincristine, Lomustine |
| ProMACE | Prednisone, Methotrexate, Leucovorin, Doxorubicin, |
| | Cyclophosphamide, Etoposide |
| ProMACE/cytaBOM | Prednisone, Doxorubicin, Cyclophosphamide, Etoposide, |
| | Cytarabine, Bleomycin, Vincristine, Methotrexate, |
| | Leucovorin, Cotrimoxazole |
| PRoMACE/MOPP | Prednisone, Doxorubicin, Cyclophosphamide, Etoposide, |
| | Mechlorethamine, Vincristine, Procarbazine, Methotrexate, |
| | Leucovorin |
| Pt/VM | Cisplatin, Teniposide |
| PVA | Prednisone, Vincristine, Asparaginase |
| PVB | Cisplatin, Vinblastine, Bleomycin |
| PVDA | Prednisone, Vincristine, Daunorubicin, Asparaginase |
| SMF | Streptozocin, Mitomycin, Fluorouracil |
| TAD | Mechlorethamine, Doxorubicin, Vinblastine, Vincristine, |
| | Bleomycin, Etoposide, Prednisone |
| TCF | Paclitaxel, Cisplatin, Fluorouracil |
| TIP | Paclitaxel, Ifosfamide, Mesna, Cisplatin |
| TTT | Methotrexate, Cytarabine, Hydrocortisone |
| Topo/CTX | Cyclophosphamide, Topotecan, Mesna |
| VAB-6 | Cyclophosphamide, Dactinomycin, Vinblastine, Cisplatin, |
| | Bleomycin |
| VAC | Vincristine, Dactinomycin, Cyclophosphamide |
| VACAdr | Vincristine, Cyclophosphamide, Doxorubicin, |
| | Dactinomycin, Vincristine |
| VAD | Vincristine, Doxorubicin, Dexamethasone |
| VATH | Vinblastine, Doxorubicin, Thiotepa, Flouxymesterone |
| VBAP | Vincristine, Carmustine, Doxorubicin, Prednisone |
| VBCMP | Vincristine, Carmustine, Melphalan, Cyclophosphamide, |
| | Prednisone |
| VC | Vinorelbine, Cisplatin |
| VCAP | Vincristine, Cyclophosphamide, Doxorubicin, Prednisone |
| VD | Vinorelbine, Doxorubicin |
| VelP | Vinblastine, Cisplatin, Ifosfamide, Mesna |
| VIP | Etoposide, Cisplatin, Ifosfamide, Mesna |
| VM | Mitomycin, Vinblastine |
| VMCP | Vincristine, Melphalan, Cyclophosphamide, Prednisone |
| VP | Etoposide, Cisplatin |
| V-TAD | Etoposide, Thioguanine, Daunorubicin, Cytarabine |
| 5 + 2 | Cytarabine, Daunorubicin, Mitoxantrone |
| 7 + 3 | Cytarabine with/, Daunorubicin or Idarubicin or |
| | Mitoxantrone |
| “8 in 1” | Methylprednisolone, Vincristine, Lomustine, |
| | Procarbazine, Hydroxyurea, Cisplatin, Cytarabine, |
| | Dacarbazine |

In certain embodiments, a compound of the invention may be conjointly administered with non-chemical methods of cancer treatment. In certain embodiments, a compound of the invention may be conjointly administered with radiation therapy. In certain embodiments, a compound of the invention may be conjointly administered with surgery, with thermoablation, with focused ultrasound therapy, or with cryotherapy.

In certain embodiments, different compounds of the invention may be conjointly administered with one or more other compounds of the invention. Moreover, such combinations may be conjointly administered with other therapeutic agents, such as other agents suitable for the treatment of a bone metabolism condition, such as the agents identified above.

In certain embodiments, the present invention provides a kit comprising: a) one or more single dosage forms of a compound of the invention; b) one or more single dosage forms of a chemotherapeutic agent as mentioned above; and c) instructions for the administration of the compound of the invention and the chemotherapeutic agent.

The present invention provides a kit comprising:
a) a pharmaceutical formulation (e.g., one or more single dosage forms) comprising a compound of the invention; and
b) instructions for the administration of the pharmaceutical formulation, e.g., for treating or preventing any of the conditions discussed above, e.g., bone loss.

In certain embodiments, the kit further comprises instructions for the administration of the pharmaceutical formulation comprising a compound of the invention conjointly with an agent suitable for the treatment or prevention of bone loss (e.g., a bisphosphonate) as mentioned above. In certain embodiments, the kit further comprises a second pharmaceutical formulation (e.g., as one or more single dosage forms) comprising an agent suitable for the treatment or prevention of bone loss (e.g., a bisphosphonate) as mentioned above.

The present invention provides a kit comprising:
a) a pharmaceutical formulation (e.g., one or more single dosage forms) comprising a compound of the invention; and
b) instructions for the administration of the pharmaceutical formulation for treating or inhibiting the development of osteoporosis, treating or inhibiting the development of periodontitis, treating or inhibiting the development of metastatic bone disease, decreasing the incidence of bone metastasis, or delaying the onset of bone metastasis.

In certain embodiments, the kit further comprises instructions for the administration of the pharmaceutical formulation comprising a compound of the invention conjointly with a chemotherapeutic agent as mentioned above. In certain embodiments, the kit further comprises a second pharmaceutical formulation (e.g., as one or more single dosage forms) comprising a chemotherapeutic agent as mentioned above.

The present invention provides a kit comprising:
a) a first pharmaceutical formulation (e.g., one or more single dosage forms) comprising an agent suitable for the treatment or prevention of bone loss (e.g., a bisphosphonate) as mentioned above; and
b) instructions for the administration of the first pharmaceutical formulation and a second pharmaceutical formulation comprising a compound of the invention for treating or preventing bone loss, treating or inhibiting the development of osteoporosis, treating or inhibiting the development of periodontitis, treating or inhibiting the development of metastatic bone disease, decreasing the incidence of bone metastasis, or delaying the onset of bone metastasis.

The present invention provides a kit comprising:
a) a first pharmaceutical formulation (e.g., one or more single dosage forms) comprising a chemotherapeutic agent as mentioned above; and
b) instructions for the administration of the first pharmaceutical formulation with a compound of the invention for treating or preventing bone loss, treating or inhibiting the development of osteoporosis, treating or inhibiting the development of periodontitis, treating or inhibiting the development of metastatic bone disease, decreasing the incidence of bone metastasis, or delaying the onset of bone metastasis.

Mucositis

The present invention provides a method of treating or preventing mucositis comprising administering a compound of the invention. Mucositis, for the purposes of this application, refers to mucosal injury induced by or associated with the administration of radiation or drugs (chemotherapy) for the treatment of cancer and related diseases. Mucositis typically manifests itself as ulcerations, tissue necrosis, and atrophy of the mucous membranes anywhere along the digestive tract, from the mouth to the anus. For example, the present methods may be used to treat ulcerations and tissue necrosis associated with radiation therapy and/or chemotherapy.

The present invention provides a method of preventing the development of chemotherapy or radiation therapy-induced mucositis comprising administering a compound of the invention. In certain embodiments, a compound of the invention is administered conjointly with chemotherapy or radiation therapy.

The present invention provides a method of improving survival rates by reducing the incidence of therapy-induced mucositis comprising administering a compound of the invention. The rate of life-threatening severe mucositis, grade 4 on WHO scale, would be expected to be reduced from an average incidence of 60% in untreated patients, to 20% or less in patents receiving a subject treatment.

In one embodiment, the method of treating or preventing mucositis may comprise administering a compound of the invention conjointly with an additional agent useful in the treatment of mucositis. In certain embodiments, the compound of the invention may be conjointly administered with an antimicrobial agent. In certain embodiments, the compound of the invention may be conjointly administered with a growth factor. In certain embodiments, the compound of the invention may be conjointly administered with an agent that inhibits the synthesis of ceramide, an agent that blocks the activity of ceramide, or an agent that degrades ceramide.

In one embodiment, the method of treating or preventing mucositis may comprise administering a compound of the invention conjointly with a chemotherapeutic agent. Chemotherapeutic agents that may be conjointly administered with compounds of the invention include any suitable chemotherapeutic agent or combination therapy as set forth above.

In certain embodiments, different compounds of the invention may be conjointly administered with one or more other compounds of the invention. Moreover, such combinations may be conjointly administered with other therapeutic agents, such as other agents suitable for the treatment or prevention of mucositis, such as the agents identified above.

In certain embodiments, the present invention provides a kit comprising: a) one or more single dosage forms of a compound of the invention; b) one or more single dosage forms of a chemotherapeutic agent as mentioned above; and c) instructions for the administration of the compound of the invention and the chemotherapeutic agent.

The present invention provides a kit comprising:
a) a pharmaceutical formulation (e.g., one or more single dosage forms) comprising a compound of the invention; and
b) instructions for the administration of the pharmaceutical formulation e.g., for treating or preventing mucositis, preventing the development of chemotherapy or radiation therapy-induced mucositis, or improving survival rates by reducing the incidence of therapy-induced mucositis.

In certain embodiments, the kit further comprises instructions for the administration of the pharmaceutical formulation comprising a compound of the invention conjointly with an agent suitable for the treatment or prevention of mucositis (e.g., an antimicrobial agent, a growth factor, an agent that inhibits the synthesis of ceramide, an agent that blocks the activity of ceramide, or an agent that degrades ceramide) as mentioned above. In certain embodiments, the kit further comprises a second pharmaceutical formulation (e.g., as one or more single dosage forms) comprising an agent suitable for the treatment of mucositis (e.g., an antimicrobial agent, a growth factor, an agent that inhibits the synthesis of ceramide, an agent that blocks the activity of ceramide, or an agent that degrades ceramide) as mentioned above.

In certain embodiments, the kit further comprises instructions for the administration of the pharmaceutical formulation comprising a compound of the invention conjointly with a chemotherapeutic agent as mentioned above. In certain embodiments, the kit further comprises a second pharmaceutical formulation (e.g., as one or more single dosage forms) comprising a chemotherapeutic agent as mentioned above.

The present invention provides a kit comprising:
a) a first pharmaceutical formulation (e.g., one or more single dosage forms) comprising an agent suitable for the treatment or prevention of mucositis (e.g., an antimicrobial agent, a growth factor, an agent that inhibits the synthesis of ceramide, an agent that blocks the activity of ceramide, or an agent that degrades ceramide) as mentioned above; and
b) instructions for the administration of the first pharmaceutical formulation with a compound of the invention, e.g., for treating or preventing mucositis, preventing the development of chemotherapy or radiation therapy-induced mucositis, or improving survival rates by reducing the incidence of therapy-induced mucositis.

The present invention provides a kit comprising:
a) a first pharmaceutical formulation (e.g., one or more single dosage forms) comprising a chemotherapeutic agent as mentioned above; and
b) instructions for the administration of the first pharmaceutical formulation with a compound of the invention, e.g., for treating or preventing mucositis, preventing the development of chemotherapy or radiation therapy-induced mucositis, or improving survival rates by reducing the incidence of therapy-induced mucositis.

Cardiovascular Disease

The present invention provides a method of treating or preventing cardiovascular disease in a patient comprising administering to said patient a compound of the invention. In certain embodiments, the method comprises optional conjoint administration with a statin.

Cardiovascular disease refers to one or more disease states of the cardiovascular tree (including the heart). Diseases of the cardiovascular tree and diseases of dependent organs include, for example, but are not limited to any one or more of:

disorders of the heart muscle (cardiomyopathy or myocarditis) such as idiopathic cardiomyopathy, metabolic cardiomyopathy which includes diabetic cardiomyopathy, alcoholic cardiomyopathy, drug-induced cardiomyopathy, ischemic cardiomyopathy, and hypertensive cardiomyopathy;
atheromatous disorders of the major blood vessels (macrovascular disease) such as the aorta, the coronary arteries, the carotid arteries, the cerebrovascular arteries, the renal arteries, the iliac arteries, the femoral arteries, and the popliteal arteries; toxic, drug-induced, and metabolic (including, but not limited to, hypertensive and/or diabetic) disorders of small blood vessels (microvascular disease) such as the retinal arterioles, the glomerular arterioles, the vasa nervorum, cardiac arterioles, and associated capillary beds of the eye, the kidney, the heart, and the central and peripheral nervous systems; and,
plaque rupture of atheromatous lesions of major blood vessels such as the aorta, the coronary arteries, the carotid arteries, the cerebrovascular arteries, the renal arteries, the iliac arteries, the fermoral arteries and the popliteal arteries.

Yet other disorders that may be treated with compounds of the invention include restenosis, e.g., following coronary intervention, and disorders relating to an abnormal level of high density and low density cholesterol.

In certain embodiments, the present invention provides methods of treating a vascular disease or disorder. In certain embodiments, the vascular disorder may include any vascular disease or disorder which comprises an autoimmune element, for example one which is caused by an autoimmune response. Exemplary vascular disorders include one or more of Raynaud's disease and phenomenon, anterior uveitis, vasculitis, obliterative vascular disorder, atheroma formation, arteriosclerosis, arteritis (e.g., Takayasu arteritis, temporal arteritis/giant cell arteritis), myointimal hyperplasia (natural or following angioplasty), inflammatory and autoimmune thickening of the intima and/or muscular layer of blood vessels, inflammatory blood vessel lesions, atherosclerotic heart disease, reperfusion injury, cardiac conduction disturbances, myocarditis, and myocardial infarction.

In certain embodiments, the present invention provides a method of treating or preventing heart attack or dysrhythmia in a patient comprising administering to said patient a compound of the invention. In certain embodiments, the present invention provides a method of preventing cardiac death in a patient comprising administering to said patient a compound of the invention. In certain embodiments, the method comprises optional conjoint administration with a statin.

Compounds of the invention are capable of resolving inflammation. Several aspects of cardiovascular disease, in particular the formation of atherosclerotic vessel wall plaques, are believed to be intimately related to inflammation. Today it is believed that serum markers of inflammation such as CRP may be as predictive of risk of cardiovascular disease as elevated levels of LDL. Thus, compounds of the invention are useful in treating or preventing cardiovascular disease. In certain embodiments, compounds of the invention are useful for the treatment or prevention of arterial inflammation and/or artherosclerosis.

Another mechanism by which compounds of the invention may be effective in treating or preventing cardiovascular disease is by inhibiting the structural and functional modifications of HDL that are an immediate effect of the acute phase response commonly seen in cardiovascular disease with active atherosclerotic vessel wall plaques. Thus, compounds of the invention may increase HDL levels (or prevent the decrease of HDL levels) and restore the LDL scavenging effects of HDL. This may lead to a lower and improved serum LDL/HDL ratio.

In addition to increasing HDL levels, statins also demonstrate anti-inflammatory activity which contributes to their ability to lower cardiovascular disease risk and treat cardiovascular disease. However, the full anti-inflammatory potential of statins cannot be utilized clinically as a monotherapy due to the high doses required, which can lead to an increased rate and severity level of treatment-limiting adverse events, notably liver toxicity.

Advantageously and surprisingly, treatment or prevention of cardiovascular disease with a combination of a statin and a compound of the invention leads to a mutual enhancement of both the anti-inflammatory properties and the serum HDL elevating properties of the two classes of compounds while avoiding the risks associated with high doses of statins alone.

In methods of the invention, wherein a compound of the invention is administered conjointly with a statin (i.e., an HMG-CoA reductase inhibitor), the statin may be chosen from any statin known in the art. Statins suitable for said conjoint administration include, but are not limited to, mevastatin ((2S)-2-methyl butanoic acid (1S,7S,8S,8aR)-1,2,3,7,8,8a-hexahydro-7-methyl-8-[2-[(2R,4R)-tetrahydro-4-hydroxy-6-oxo-2H-pyran-2-yl]ethyl]-1-naphthalenyl ester), atorvastatin ((βR,δR)-2-(4-fluorophenyl)-β,δ-dihydroxy-5-(1-methylethyl)-3-phenyl-4-[(phenylamino)carbonyl]-1H-Pyrrole-1-heptanoic acid), fluvastatin ((3R,5S,6E)-rel-7-[3-(4-fluorophenyl)-1-(1-methylethyl)-1H-indol-2-yl]-3,5-dihydroxy-6-heptenoic acid), lovastatin (2(S)-2-methyl-butanoic acid (1S,3R,7S,8S,8aR)-1,2,3,7,8,8a-hexahydro-3,7-dimethyl-8-[2-[(2R,4R)-tetrahydro-4-hydroxy-6-oxo-2H-pyran-2-yl]ethyl]-1-naphthalenyl ester), pravastatin ((βR,δR,1S,2S,6S,8S,8aR)-1,2,6,7,8,8a-hexahydro-β,β,6-trihydroxy-2-methyl-8-[(2S)-2-methyl-1-oxobutoxy]-1-naphthaleneheptanoic acid), simvastatin (2,2-dimethyl-butanoic acid (1S,3R,7S,8S,8aR)-1,2,3,7,8,8a-hexahydro-3,7-dimethyl-8-[2-[(2R,4R)-tetrahydro-4-hydroxy-6-oxo-2H-pyran-2-yl]ethyl]-1-naphthalenyl ester), rosuvastatin ((3R,5S,6E)-7-[4-(4-fluorophenyl)-6-(1-methylethyl)-2-[methyl(methylsulfonyl)amino]-5-pyrimidinyl]-3,5-dihydroxy-6-heptenoic acid), eptastatin, pitavastatin ((3R,5S,6E)-7-[2-cyclopropyl-4-(4-fluorophenyl)-3-quinolinyl]-3,5-dihydroxy-6-heptenoic acid), cerivastatin ((3R,5S,6E)-7-[4-(4-fluorophenyl)-5-(methoxymethyl)-2,6-bis(1-methylethyl)-3-pyridinyl]-3,5-dihydroxy-6-heptenoic acid), berivastatin ((R*,S*-(E)-7-(4-(4-fluorophenyl)spiro(2H-1-benzopyran-2,1′-cyclopentan)-3-yl)-3,5-dihydroxy-ethyl ester), dalvastatin ((4R,6S)-rel-6-[(1E)-2-[2-(4-fluoro-3-methylphenyl)-4,4,6,6-tetramethyl-1-cyclohexen-1-yl]ethenyl]tetrahydro-4-hydroxy-, 2H-Pyran-2-one), glenvastatin ((4R,6S)-6-[(1E)-2-[4-(4-fluorophenyl)-2-(1-methylethyl)-6-phenyl-3-pyridinyl]ethenyl]tetrahydro-4-hydroxy-2H-Pyran-2-one), RP 61969 ([2S-[2a(E),4β]]-; 4-(4-fluorophenyl)-2-(1-methylethyl)-3-[2-(tetrahydro-4-hydroxy-6-oxo-2H-pyran-2-yl)ethenyl]-1(2H)-isoquinolinone), SDZ-265859, BMS-180431 ((3R,5S,6E)-rel-9,9-bis(4-fluorophenyl)-3,5-dihydroxy-8-(1-methyl-1H-tetrazol-5-yl)-6,8-Nonadienoic acid), CP-83101 ((3R,5S,6E)-rel-3,5-dihydroxy-9,9-diphenyl-6,8-Nonadienoic acid methyl ester), dihydromevinolin ((2S)-2-methyl-butanoic acid (1S,3S,4aR,7S,8S,8aS)-1,2,3,4,4a,7,8,8a-octahydro-3,7-dimethyl-8-[2-[(2R,4R)-tetrahydro-4-hydroxy-6-oxo-2H-pyran-2-yl]ethyl]-1-naphthalenyl ester), and L-669262 (2,2-dimethyl-butanoic acid (1S,7R,8R,8aR)-1,2,6,7,8,8a-hexahydro-3,7-dimethyl-6-oxo-8-[2-[(2R,4R)-tetrahydro-4-hydroxy-6-oxo-2H-pyran-2-yl]ethyl]-1-naphthalenyl ester).

For example, statins suitable for use in the methods of this invention include statins of formula 200:

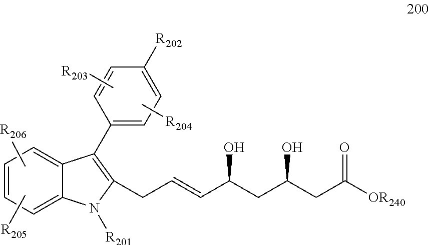

wherein
R₂₀₁ is selected from alkyl, alkenyl, alkynyl, cycloalkyl or aralkyl;
R₂₀₂, R₂₀₃ and R₂₀₄ are independently selected from hydrogen, halogen, alkyl, alkenyl or alkynyl;
R₂₀₅ and R₂₀₆ are independently selected from hydrogen, halogen, alkyl, alkenyl, alkynyl, cycloalkyl, aralkyl, alkoxy or aralkoxy; and
R₂₄₀ is selected from hydrogen, R₂₄₁ or M;
R₂₄₁ is a physiologically acceptable and hydrolyzable ester group; and
M is a pharmaceutically acceptable cation;
or enantiomers or salts or hydrates thereof.

Other statins suitable for use in the methods of this invention include statins of formula 201:

A-B

wherein
A is selected from

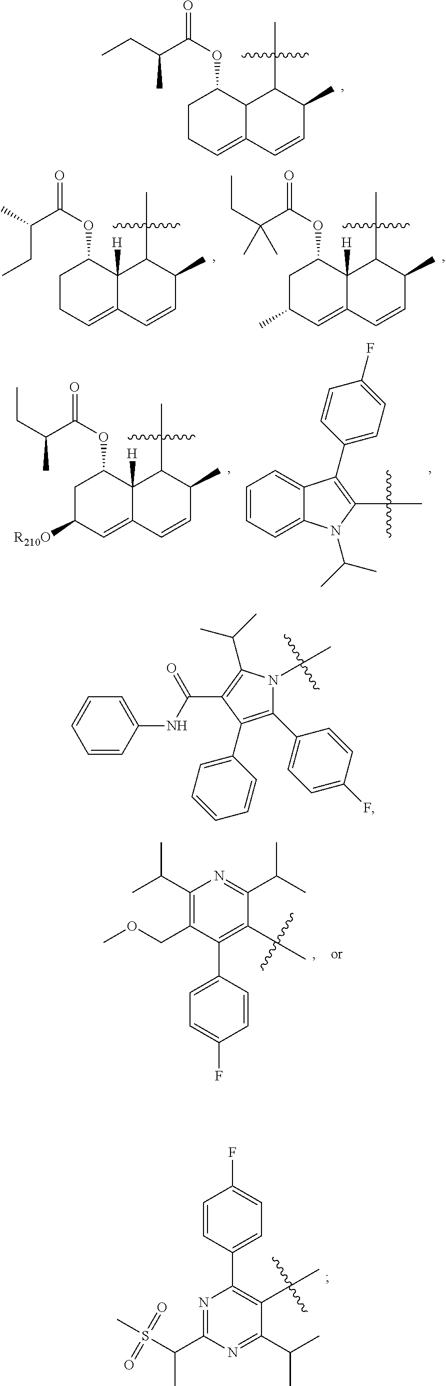

B is selected from

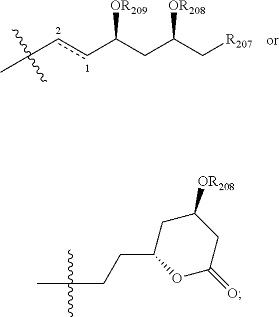

C1 and C2 are joined by a single or a double bond;
R₂₀₇ is selected from CO₂R₂₁₅, CONR₂₁₁R₂₁₂ or CH₂OR₂₁₃, or R₂₀₇ and R₂₀₉ can form a lactone;
R₂₁₅ is selected from H or a cationic salt moiety, or CO₂R₂₁₅ forms a pharmaceutically acceptable ester moiety;
R₂₀₈, R₂₀₉ and R₂₁₀ are independently selected from H, C(O)R₂₁₄ or C(O)NR₂₁₁R₂₁₂;
R₂₁₁ and R₂₁₂ are independently selected from H, alkyl, alkenyl or alkynyl;
R₂₁₃ is selected from H or C(O)R₂₁₄; and
R₂₁₄ is selected from alkyl, alkenyl or alkynyl.

Other statins suitable for use in the methods of this invention include statins of formula 202:

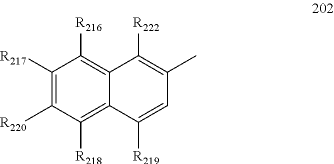

wherein
R₂₂₂ is selected from

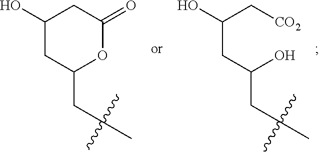

R₂₁₆ is selected from OH, C₆H₅CO₂ or R₂₂₁CO₂;
R₂₂₁ is a branched or straight C₁-C₅ alkyl, C₂-C₅ alkenyl, or C₂-C₅ alkynyl;
R₂₁₇, R₂₁₈ and R₂₁₉ are independently selected from H, C₁-C₅ alkyl, C₂-C₅ alkenyl, C₂-C₅ alkynyl or C₁-C₅ acyl; and
R₂₂₀ is selected from H or CH₃.

Other statins suitable for use in the methods of this invention include statins of formula 203:

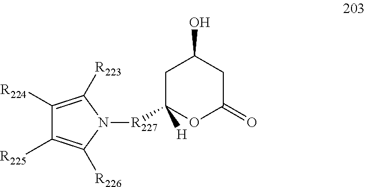

wherein
R₂₂₇ is —CH₂—, —CH₂—CH₂—, —CH₂—CH₂—CH₂— or —CH₂—CH(CH₃)—;
R₂₂₃ is 1-naphthyl; 2-naphthyl; cyclohexyl; norbornenyl; 2-, 3-, or 4-pyridinyl; phenyl, phenyl substituted with fluorine, chlorine, bromine, hydroxyl; trifluoromethyl; alkyl, alkenyl, or alkynyl of from one to four carbon atoms, alkoxy of from one to four carbon atoms, or alkanoyloxy of from two to eight carbon atoms;
Either R₂₂₄ or R₂₂₅ is —CONR₂₂₈R₂₂₉ where R₂₂₈ and R₂₂₉ are independently hydrogen; alkyl, alkenyl, or alkynyl of from one to six carbon atoms; 2-, 3-, or 4-pyridinyl; phenyl; phenyl substituted with fluorine, chlorine, bromine, cyano, trifluoromethyl, or carboalkoxy of from three to eight carbon atoms; and the other of R₂₂₄ or R₂₂₅ is hydrogen; alkyl, alkenyl, or alkynyl of from one to six carbon atoms; cyclopropyl; cyclobutyl; cyclopentyl; cyclohexyl; phenyl; or phenyl substituted with fluorine, chlorine, bromine, hydroxyl; trifluoromethyl; alkyl, alkenyl, or alkynyl of from one to four carbon atoms, alkoxy of from one to four carbon atoms, or alkanoyloxy of from two to eight carbon atoms; and
R₂₂₆ is alkyl, alkenyl, or alkynyl of from one to six carbon atoms; cyclopropyl; cyclobutyl; cyclopentyl; cyclohexyl; or trifluoromethyl;
or the hydroxyl acids, and pharmaceutically acceptable salts thereof, derived from the opening of the lactone ring.

Other statins suitable for use in the methods of this invention include statins of formula 204:

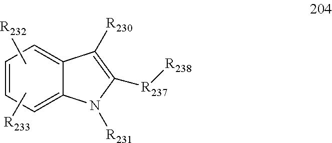

wherein
one of R₂₃₀ and R₂₃₁ is

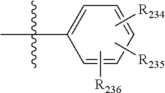

and the other is primary or secondary C₁₋₆ alkyl, alkenyl, or alkynyl not containing an asymmetric carbon atom, C₃₋₆ cycloalkyl or phenyl-(CH₂)ₘ—;
R₂₃₄ is selected from hydrogen, C₁₋₃ alkyl, C₂-C₄ alkenyl, C₂-C₄ alkynyl, n-butyl, i-butyl, t-butyl, C₁₋₃ alkoxy, n-butoxy, i-butoxy, trifluoromethyl, fluoro, chloro, phenoxy or benzyloxy;
R₂₃₅ is selected from hydrogen, C₁₋₃ alkyl, C₂-C₃ alkenyl, C₂-C₃ alkynyl, C₁₋₃ alkoxy, trifluoromethyl, fluoro, chloro, phenoxy or benzyloxy;
R₂₃₆ is selected from hydrogen, C₁₋₂ alkyl, C₂ alkenyl, C₂ alkynyl, C₁₋₂ alkoxy, fluoro or chloro;
m is selected from 1, 2 or 3, with the provisos that both R₂₃₅ and R₂₃₆ must be hydrogen when R₂₃₄ is hydrogen, R₂₃₆ must be hydrogen when R₂₃₅ is hydrogen, not more than one of R₂₃₄ and R₂₃₅ is trifluoromethyl, not more than one of R₂₃₄ and R₂₃₅ is phenoxy, and not more than one of R₂₃₄ and R₂₃₅ is benzyloxy;
R₂₃₂ is selected from hydrogen, C₁₋₃ alkyl, C₂-C₄ alkenyl, C₂-C₄ alkynyl, n-butyl, i-butyl, t-butyl, C₃₋₆ cycloalkyl, C₁₋₃ alkoxy, n-butoxy, i-butoxy, trifluoromethyl, fluoro, chloro, phenoxy or benzyloxy;
R₂₃₃ is selected from hydrogen, C₁₋₃ alkyl, C₂-C₃ alkenyl, C₂-C₃ alkynyl, C₁₋₃ alkoxy, trifluoromethyl, fluoro, chloro, phenoxy or benzyloxy, with the provisos that R₂₃₃ must be hydrogen when R₂₃₂ is hydrogen, not more than one of R₂₃₂ and R₂₃₃ is trifluoromethyl, not more than one of R₂₃₂ and R₂₃₃ is phenoxy, and not more than one of R₂₃₂ and R₂₃₃ is benzyloxy;
R₂₃₇ is selected from —(CH₂)ₙ— or —CH═CH—, wherein n is 0, 1, 2 or 3;
R₂₃₈ is selected from

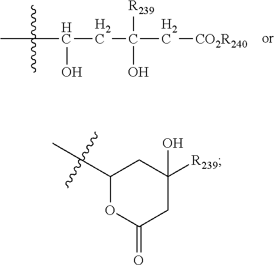

R₂₃₉ is selected from hydrogen, or C₁₋₃ alkyl, C₂-C₃ alkenyl, or C₂-C₃ alkynyl;
R₂₄₀ is selected from hydrogen, R₂₄₁ or M;
R₂₄₁ is a physiologically acceptable and hydrolyzable ester group; and
M is a pharmaceutically acceptable cation.

Other statins suitable for use in the methods of this invention include statins of formula 205:

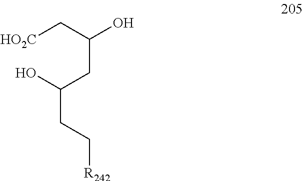

wherein
R₂₄₂ is selected from

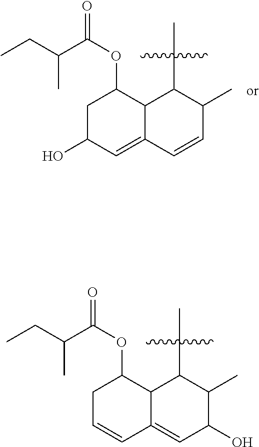

or ring-closed lactones, salts or esters thereof.

Other statins suitable for use in the methods of this invention include statins of formula 206:

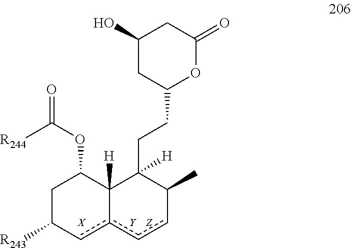

wherein
R₂₄₃ is selected from H or CH₃;
R₂₄₄ is selected from 1,1-dimethylpropyl; C₃₋₁₀cycloalkyl; C₂₋₁₀alkenyl; C₁₋₁₀CF₃-substituted alkyl; phenyl; halophenyl; phenyl-C₁₋₃alkyl; substituted phenyl-C₁₋₃alkyl in which the substituent is halo, C₁₋₃alkyl or C₁₋₃alkoxy;
the dotted lines at X, Y and Z represent possible double bonds, said double bonds, when any are present, being either X and Z in combination or X, Y or Z alone;
or the corresponding dihydroxy acid of formula 206a

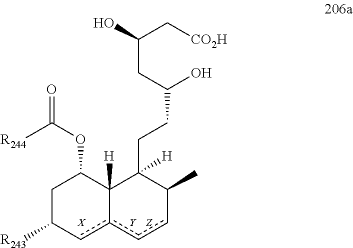

or a pharmaceutically acceptable salt of said acid, a C₁₋₄alkyl ester of said acid or a phenyldimethylamino-, or acetylamino-substituted-C₁₋₄alkyl ester of said acid.

Other statins suitable for use in the methods of this invention include statins of formula 207:

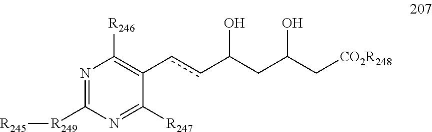

wherein
R₂₄₅ is lower alkyl, alkenyl, alkynyl, aryl or aralkyl, each of which may have one or more substituents;
R₂₄₆ and R₂₄₇ independently are selected from hydrogen, lower alkyl, alkenyl, alkynyl, or aryl, and each of said lower alkyl, alkenyl, alkynyl and aryl may have one or more substituents;
R₂₄₈ is hydrogen, lower alkyl, alkenyl, alkynyl, or a cation capable of forming a non-toxic pharmaceutically acceptable salt;
R₂₄₉ is sulfur, oxygen, or sulfonyl, or imino which may have a substituent; and
the dotted line represents the presence or absence of a double bond;
or the corresponding ring-closed lactone.

The synthesis of various statins is set forth in US RE37314 E, U.S. Pat. No. 4,444,784, U.S. Pat. No. 4,346,227, U.S. Pat. No. 5,354,772, U.S. Pat. No. 4,681,893 and US 2005/0228042.

In another embodiment, the invention provides a method of raising serum HDL concentration (or preventing a decrease in serum HDL concentration) or decreasing the serum LDL/HDL ratio in a patient, said method comprising administering to said patient a compound of the invention, optionally in combination with a statin. The patient to be treated in this method may have a total serum cholesterol level of greater than 189 mg/dl, preferably higher than 200 mg/dl and most preferably higher than 240 mg/dl; and/or a serum LDL concentration of greater than 130 mg/dl, preferably greater than 160 mg/dl, and most preferably higher than 189 mg/dl. In addition to serum cholesterol and/or LDL levels, other factors to be considered are the presence or absence of coronary disease and risk factors, such as age (45 or over for men, 55 or over for women), family history of coronary heart disease, smoking, high blood pressure, serum HDL cholesterol level, or presence of diabetes.

In certain embodiments, the invention provides a method of lowering triglyceride levels in a patient, said method comprising administering to said patient a compound of the invention, optionally in combination with a statin.

In certain embodiments, the patient to be treated in this method of the invention may already be receiving a cholesterol-lowering drug. In one preferred embodiment, the patient is already taking a statin, such as one of the statins described above; and will continue to take that drug conjointly with a compound of the invention. Alternatively, the compound of the invention may be used as a replacement for the previously administered cholesterol-lowering drug.

In a related embodiment, the invention provides a method of reducing the dose of a statin required to achieve a desired increase in serum HDL, a decrease in serum LDL/HDL ratio or serum total cholesterol level, and/or a decrease in triglyceride level. Reducing the dose of statins while maintaining potent serum lipid-reducing properties is highly desirable due to side effects associated with certain statins. Well-known side effects include, deleterious changes in liver function, muscle pain, weakness, muscle tenderness, myopathy. Other side effects of statins include reduced cognition, memory impairment, depression, irritability, non-muscle pain, peripheral neuropathy, sleep disorders, sexual dysfunction, fatigue, dizziness, swelling, shortness of breath, vision changes, changes in temperature regulation, weight change, hunger, breast enlargement, blood sugar changes, dry skin, rashes, blood pressure changes, nausea, upset stomach, bleeding, and ringing in ears or other noises.

In this embodiment, the dose of a statin is reduced by at least 5%, at least 10%, at least 15%, at least 20%, at least 25%, at least 30%, at least 40%, at least 50%, at least 60%, at least 70%, or more. The actual reduction in statin dose will depend upon the nature of the compound of the invention being administered, the amount of compound of the invention being administered, and the reduction in serum lipid and/or triglyceride level desired, as well as other factors set forth elsewhere in this application that are typically considered in treating a disease or condition. The amount of compound of the invention administered in this method will also depend upon the factors set forth above, as well as the nature and amount of statin being administered. In certain embodiments, the amount of compound of the invention administered in this method is less than 5%, less than 10%, less than 15%, less than 20%, less than 25%, less than 30%, less than 40%, less than 50%, less than 60%, less than 70%, less than 80%, or less than 90% of the dose of compound of the invention required to produce an anti-inflammatory effect. In other embodiments, the amount of compound of the invention administered is over 110%, over 120%, over 130%, over 140%, over 150%, over 160%, over 170%, over 180%, over 190%, or even over 200% of the dose of compound of the invention required to produce an anti-inflammatory effect.

In one embodiment, the method of treating or preventing cardiovascular disease according to this invention may comprise the additional step of conjointly administering to the patient another agent suitable for treating cardiovascular disease, such as, for example, a cyclooxygenase inhibitor, a thromboxane receptor antagonist, a prostacyclin mimetic, a phosphodiesterase inhibitor, a vasodilator, a cerebral protecting drug, a brain metabolic stimulant, an anticoagulant, an antiplatelet drug, a thrombolytic drug, an antihypertensive agent, a calcium channel blocker, an antianginal drug, a diuretic, a cardioplegic solution, a cardiotonic agent, an antiarrhythmic drug, a fibrinolytic agent, a sclerosing solution, a vasoconstrictor agent, a nitric oxide donor, a potassium channel blocker, a sodium channel blocker, an antihyperlipidemic drug, an immunosuppressant, or a natriuretic agent.

Examples of a cyclooxygenase inhibitor include, but are not limited to, celecoxib, rofecoxib, meloxicam, valdecoxib, aspirin or indomethacin.

An example of a thromboxane receptor antagonist is ifetroban.

Examples of vasodilators include, e.g., bencyclane, cinnarizine, citicoline, cyclandelate, cyclonicate, ebumamonine, phenoxezyl, flunarizine, ibudilast, ifenprodil, lomerizine, naphlole, nikamate, nosergoline, nimodipine, papaverine, pentifylline, nofedoline, vincamin, vinpocetine, vichizyl, pentoxifylline, prostacyclin derivatives (such as prostaglandin E1 and prostaglandin I2), an endothelin receptor blocking drug (such as bosentan), diltiazem, nicorandil, and nitroglycerin.

Examples of the cerebral protecting drug include radical scavengers (such as edaravone, vitamin E, and vitamin C), glutamate antagonists, AMPA antagonists, kainate antagonists, NMDA antagonists, GABA agonists, growth factors, opioid antagonists, phosphatidylcholine precursors, serotonin agonists, Na⁺/Ca²⁺ channel inhibitory drugs, and K⁺ channel opening drugs.

Examples of the brain metabolic stimulants include amantadine, tiapride, and gamma-aminobutyric acid.

Examples of the anticoagulant include heparins (such as heparin sodium, heparin potassium, dalteparin sodium, dalteparin calcium, heparin calcium, parnaparin sodium, reviparin sodium, and danaparoid sodium), warfarin, enoxaparin, argatroban, batroxobin, and sodium citrate.

Examples of the antiplatelet drug include ticlopidine hydrochloride, dipyridamole, cilostazol, ethyl icosapentate, sarpogrelate hydrochloride, dilazep hydrochloride, trapidil, a nonsteroidal antiinflammatory agent (such as aspirin), beraprostsodium, iloprost, and indobufene.

Examples of the thrombolytic drug include urokinase, tissue plasminogen activator (tPA), recombinant tPA, issue-type plasminogen activators (such as alteplase, tisokinase, nateplase, pamiteplase, monteplase, and rateplase), streptokinase, urokinase, prourokinase, anisoylated plasminogen streptokinase activator complex (APSAC, Eminase, Beecham Laboratories), animal salivary gland plasminogen activators, and nasaruplase.

Examples of the antihypertensive drug include angiotensin converting enzyme inhibitors (such as captopril, alacepril, lisinopril, imidapril, quinapril, temocapril, delapril, benazepril, cilazapril, trandolapril, enalapril, ceronapril, fosinopril, imadapril, mobertpril, perindopril, ramipril, spirapril, zofenopril, pentopril, randolapril and salts of such compounds), angiotensin II antagonists (such as losartan, candesartan, valsartan, eprosartan, and irbesartan), calcium channel blocking drugs (such as aranidipine, efonidipine, nicardipine, bamidipine, benidipine, manidipine, cilnidipine, nisoldipine, nitrendipine, nifedipine, nilvadipine, felodipine, amlodipine, diltiazem, bepridil, clentiazem, phendilin, galopamil, mibefradil, prenylamine, semotiadil, terodiline, verapamil, cilnidipine, elgodipine, isradipine, lacidipine, lercanidipine, nimodipine, cinnarizine, flunarizine, lidoflazine, lomerizine, bencyclane, etafenone, and perhexyline), β-adrenaline receptor blocking drugs (propranolol, pindolol, indenolol, carteolol, bunitrolol, atenolol, acebutolol, metoprolol, timolol, nipradilol, penbutolol, nadolol, tilisolol, carvedilol, bisoprolol, betaxolol, celiprolol, bopindolol, bevantolol, labetalol, alprenolol, amosulalol, arotinolol, befunolol, bucumolol, bufetolol, buferalol, buprandolol, butylidine, butofilolol, carazolol, cetamolol, cloranolol, dilevalol, epanolol, levobunolol, mepindolol, metipranolol, moprolol, nadoxolol, nevibolol, oxprenolol, practol, pronetalol, sotalol, sufinalol, talindolol, tertalol, toliprolol, xybenolol, and esmolol), α-receptor blocking drugs (such as amosulalol, prazosin, terazosin, doxazosin, bunazosin, urapidil, phentolamine, arotinolol, dapiprazole, fenspiride, indoramin, labetalol, naftopidil, nicergoline, tamsulosin, tolazoline, trimazosin, and yohimbine), sympathetic nerve inhibitors (such as clonidine, guanfacine, guanabenz, methyldopa, and reserpine), hydralazine, todralazine, budralazine, and cadralazine.

Examples of the antianginal drug include nitrate drugs (such as amyl nitrite, nitroglycerin, and isosorbide), β-adrenaline receptor blocking drugs (exemplified above), calcium channel blocking drugs (exemplified above) trimetazidine, dipyridamole, etafenone, dilazep, trapidil, nicorandil, enoxaparin, and aspirin.

Examples of the diuretic include thiazide diuretics (such as hydrochlorothiazide, methyclothiazide, bendrofluazide, chlorothiazide, trichlormethiazide, benzylhydrochlorothiazide, flumethiazide, hydroflumethiazide, bendroflumethiazide, methylchlorthiazide, polythiazide, benzthiazide and penflutizide), loop diuretics (such as furosemide, etacrynic acid, bumetanide, piretanide, azosemide, and torasemide), K⁺ sparing diuretics (spironolactone, triamterene, amiloride, and potassium canrenoate), osmotic diuretics (such as isosorbide, D-mannitol, and glycerin), nonthiazide diuretics (such as meticrane, tripamide, chlorthalidone, and mefruside), and acetazolamide.

Examples of the cardiotonic include digitalis formulations (such as digitoxin, digoxin, methyldigoxin, deslanoside, vesnarinone, lanatoside C, and proscillaridin), xanthine formulations (such as aminophylline, choline theophylline, diprophylline, and proxyphylline), catecholamine formulations (such as dopamine, dobutamine, and docarpamine), PDE III inhibitors (such as aminone, olprinone, and milrinone), denopamine, ubidecarenone, pimobendan, levosimendan, aminoethylsulfonic acid, vesnarinone, carperitide, and colforsin daropate.

Examples of the antiarrhythmic drug include ajmaline, pirmenol, procainamide, cibenzoline, disopyramide, quinidine, aprindine, mexiletine, lidocaine, phenyloin, pilsicamide, propafenone, flecamide, atenolol, acebutolol, sotalol, propranolol, metoprolol, pindolol, amiodarone, nifekalant, diltiazem, bepridil, moricizine, tocamide, encamide, propafenone, esmolol, artilide, bretylium, clofilium, isobutilide, sotalol, azimilide, dofetilide, dronedarone, ersentilide, ibutilide, tedisamil, trecetilide, digitalis, adenosine, nickel chloride, and magnesium ions and verapamil.

Examples of the antihyperlipidemic drug include atorvastatin, simvastatin, pravastatin sodium, fluvastatin sodium, clinofibrate, clofibrate, simfibrate, fenofibrate, bezafibrate, colestimide, colestyramine, mevastatin ((2S)-2-methyl butanoic acid (1S,7S,8S,8aR)-1,2,3,7,8,8a-hexahydro-7-methyl-8-[2-[(2R,4R)-tetrahydro-4-hydroxy-6-oxo-2H-pyran-2-yl]ethyl]-1-naphthalenyl ester), fluvastatin ((3R,5S,6E)-rel-7-[3-(4-fluorophenyl)-1-(1-methylethyl)-1H-indol-2-yl]-3,5-dihydroxy-6-heptenoic acid), lovastatin (2(S)-2-methyl-butanoic acid (1S,3R,7S,8S,8aR)-1,2,3,7,8,8a-hexahydro-3,7-dimethyl-8-[2-[(2R,4R)-tetrahydro-4-hydroxy-6-oxo-2H-pyran-2-yl]ethyl]-1-naphthalenyl ester), pravastatin ((βR,δR,1S,2S,6S,8S,8aR)-1,2,6,7,8,8a-hexahydro-β,β,6-trihydroxy-2-methyl-8-[(2S)-2-methyl-1-oxobutoxy]-1-naphthaleneheptanoic acid), rosuvastatin ((3R,5S,6E)-7-[4-(4-fluorophenyl)-6-(1-methylethyl)-2-[methyl(methylsulfonyl)amino]-5-pyrimidinyl]-3,5-dihydroxy-6-heptenoic acid), eptastatin, pitavastatin ((3R,5S,6E)-7-[2-cyclopropyl-4-(4-fluorophenyl)-3-quinolinyl]-3,5-dihydroxy-6-heptenoic acid), cerivastatin ((3R,5S,6E)-7-[4-(4-fluorophenyl)-5-(methoxymethyl)-2,6-bis(1-methylethyl)-3-pyridinyl]-3,5-dihydroxy-6-heptenoic acid), berivastatin ((R*,S*-(E)-7-(4-(4-fluorophenyl)spiro(2H-1-benzopyran-2,1′-cyclopentan)-3-yl)-3,5-dihydroxy-ethyl ester), dalvastatin ((4R,6S)-rel-6-[(1E)-2-[2-(4-fluoro-3-methylphenyl)-4,4,6,6-tetramethyl-1-cyclohexen-1-yl]ethenyl]tetrahydro-4-hydroxy-, 2H-Pyran-2-one), glenvastatin ((4R,6S)-6-[(1E)-2-[4-(4-fluorophenyl)-2-(1-methylethyl)-6-phenyl-3-pyridinyl]ethenyl]tetrahydro-4-hydroxy-2H-Pyran-2-one), RP 61969 ([2S-[2a(E),4β]]-; 4-(4-fluorophenyl)-2-(1-methylethyl)-3-[2-(tetrahydro-4-hydroxy-6-oxo-2H-pyran-2-yl)ethenyl]-1(2H)-isoquinolinone), SDZ-265859, BMS-180431 ((3R,5S,6E)-rel-9,9-bis(4-fluorophenyl)-3,5-dihydroxy-8-(1-methyl-1H-tetrazol-5-yl)-6,8-Nonadienoic acid), CP-83101 ((3R,5S,6E)-rel-3,5-dihydroxy-9,9-diphenyl-6,8-Nonadienoic acid methyl ester), dihydromevinolin ((2S)-2-methyl-butanoic acid (1S,3S,4aR,7S,8S,8aS)-1,2,3,4,4a,7,8,8a-octahydro-3,7-dimethyl-8-[2-[(2R,4R)-tetrahydro-4-hydroxy-6-oxo-2H-pyran-2-yl]ethyl]-1-naphthalenyl ester), and L-669262 (2,2-dimethyl-butanoic acid (1S,7R,8R,8aR)-1,2,6,7,8,8a-hexahydro-3,7-dimethyl-6-oxo-8-[2-[(2R,4R)-tetrahydro-4-hydroxy-6-oxo-2H-pyran-2-yl]ethyl]-1-naphthalenyl ester).

Examples of the immunosuppressant include azathioprine, mizoribine, cyclosporine, tacrolimus, gusperimus, and methotrexate.

In one embodiment, the method of treating or preventing cardiovascular disease according to this invention may comprise administering a compound of the invention conjointly with non-chemical means for the treatment or prevention of cardiovascular disease, such as part of a regimen including physical intervention (e.g., percutaneous transluminal coronary angioplasty, coronary surgery, or vascular surgery). In certain such embodiments, the regimen including physical intervention may further include conjointly administering another agent suitable for the treatment or prevention of cardiovascular disease, such as the agents listed above.

It should be understood that the methods of treatment or prevention of cardiovascular disease according to this invention may include conjointly administering one or more of the above agents either as a separate dosage form or as part of a composition that also comprises a statin, a compound of the invention, and optionally further comprising a statin. Moreover, the use of a composition comprising both a statin and a compound of the invention according to this invention in the treatment of cardiovascular disease, does not preclude the separate but conjoint administration of another statin.

The method of increasing serum HDL concentration, reducing serum LDL/HDL ratio, reducing total serum cholesterol concentration, and/or lowering the triglyceride level in a patient according to this invention may additionally comprise administering to said patient another active ingredient other than a statin. Such additional active ingredient may be selected from a non-statin cholesterol lowering reagent, such as bile acid sequestrants (colesevelam, cholestyramine and colestipol), niacin, fibrates (gemfibrozil, probucol and clofibrate).

In certain embodiments, different compounds of the invention may be conjointly administered with one or more other compounds of the invention. Moreover, such combinations may be conjointly administered with other therapeutic agents, such as other agents suitable for the treatment or prevention of cardiovascular disease, such as the agents identified above.

In certain embodiments, the present invention provides a kit comprising:
a) one or more single dosage forms of a compound of the invention;
b) one or more single dosage forms of a statin as mentioned above; and
c) instructions for the administration of the compound of the invention and the statin.

The present invention provides a kit comprising:
a) a pharmaceutical formulation (e.g., one or more single dosage forms) comprising a compound of the invention; and
b) instructions for the administration of the pharmaceutical formulation e.g., for treating or preventing cardiovascular disease, raising serum HDL concentration (or preventing a decrease in serum HDL concentration), decreasing the serum LDL/HDL ratio, and/or lowering triglyceride levels.

In certain embodiments, the kit further comprises instructions for the administration of the pharmaceutical formulation comprising a compound of the invention conjointly with an agent suitable for treating or preventing cardiovascular disease, raising serum HDL concentration (or preventing a decrease in serum HDL concentration), decreasing the serum LDL/HDL ratio, and/or lowering triglyceride levels (e.g., a statin or another agent suitable for treating cardiovascular disease, such as, for example, a cyclooxygenase inhibitor, a thromboxane receptor antagonist, a prostacyclin mimetic, a phosphodiesterase inhibitor, a vasodilator, a cerebral protecting drug, a brain metabolic stimulant, an anticoagulant, an antiplatelet drug, a thrombolytic drug, an antihypertensive agent, a calcium channel blocker, an antianginal drug, a diuretic, a cardioplegic solution, a cardiotonic agent, an antiarrhythmic drug, a fibrinolytic agent, a sclerosing solution, a vasoconstrictor agent, a nitric oxide donor, a potassium channel blocker, a sodium channel blocker, an antihyperlipidemic drug, an immunosuppressant, or a natriuretic agent) as mentioned above. In certain embodiments, the kit further comprises a second pharmaceutical formulation (e.g., as one or more single dosage forms) comprising an agent suitable for treating or preventing cardiovascular disease, raising serum HDL concentration (or preventing a decrease in serum HDL concentration), decreasing the serum LDL/HDL ratio, and/or lowering triglyceride levels (e.g., a statin or another agent suitable for treating cardiovascular disease, such as, for example, a cyclooxygenase inhibitor, a thromboxane receptor antagonist, a prostacyclin mimetic, a phosphodiesterase inhibitor, a vasodilator, a cerebral protecting drug, a brain metabolic stimulant, an anticoagulant, an antiplatelet drug, a thrombolytic drug, an antihypertensive agent, a calcium channel blocker, an antianginal drug, a diuretic, a cardioplegic solution, a cardiotonic agent, an antiarrhythmic drug, a fibrinolytic agent, a sclerosing solution, a vasoconstrictor agent, a nitric oxide donor, a potassium channel blocker, a sodium channel blocker, an antihyperlipidemic drug, an immunosuppressant, or a natriuretic agent) as mentioned above.

The present invention provides a kit comprising:
a) a first pharmaceutical formulation (e.g., one or more single dosage forms) comprising an agent suitable for treating or preventing cardiovascular disease, raising serum HDL concentration (or preventing a decrease in serum HDL concentration), decreasing the serum LDL/HDL ratio, and/or lowering triglyceride levels (e.g., a statin or another agent suitable for treating cardiovascular disease, such as, for example, a cyclooxygenase inhibitor, a thromboxane receptor antagonist, a prostacyclin mimetic, a phosphodiesterase inhibitor, a vasodilator, a cerebral protecting drug, a brain metabolic stimulant, an anticoagulant, an antiplatelet drug, a thrombolytic drug, an antihypertensive agent, a calcium channel blocker, an antianginal drug, a diuretic, a cardioplegic solution, a cardiotonic agent, an antiarrhythmic drug, a fibrinolytic agent, a sclerosing solution, a vasoconstrictor agent, a nitric oxide donor, a potassium channel blocker, a sodium channel blocker, an antihyperlipidemic drug, an immunosuppressant, or a natriuretic agent); and
b) instructions for the administration of the first pharmaceutical formulation with a compound of the invention, e.g., for treating or preventing cardiovascular disease, raising serum HDL concentration (or preventing a decrease in serum HDL concentration), decreasing the serum LDL/HDL ratio, and/or lowering triglyceride levels.

The present invention provides a kit comprising:
a) a first pharmaceutical formulation (e.g., one or more single dosage forms) comprising a statin as mentioned above; and
b) instructions for the administration of the first pharmaceutical formulation with a compound of the invention, e.g., for treating or preventing cardiovascular disease, raising serum HDL concentration (or preventing a decrease in serum HDL concentration), decreasing the serum LDL/HDL ratio, and/or lowering triglyceride levels.

### General Inflammatory Diseases

The present invention provides a method of treating or preventing inflammatory disease in a patient comprising administering to said patient a compound of the invention.

Examples of inflammatory conditions, which may be treated or prevented by the administration of a compound of the invention include, but are not limited to, inflammation of the lungs, joints, connective tissue, eyes, nose, bowel, kidney, liver, skin, central nervous system, vascular system and heart. In certain embodiments, inflammatory conditions which may be treated by the current invention include inflammation due to the infiltration of leukocytes or other immune effector cells into affected tissue. Other relevant examples of inflammatory conditions which may be treated by the present invention include inflammation caused by infectious agents, including, but not limited to, viruses, bacteria fungi and parasites.

Inflammatory lung conditions include, but are not limited to, asthma, adult respiratory distress syndrome, bronchitis, pulmonary inflammation, pulmonary fibrosis, and cystic fibrosis (which may additionally or alternatively involve the gastro-intestinal tract or other tissue(s)). Inflammatory joint conditions include rheumatoid arthritis, rheumatoid spondylitis, juvenile rheumatoid arthritis, osteoarthritis, gouty arthritis and other arthritic conditions. Eye diseases with an inflammatory component include, but are not limited to, uveitis (including iritis), conjunctivitis, scleritis, keratoconjunctivitis sicca, and retinal diseases, including, but not limited to, diabetic retinopathy, retinopathy of prematurity, retinitis pigmentosa, and dry and wet age-related macular degeneration. Inflammatory bowel conditions include Crohn's disease, ulcerative colitis and distal proctitis.

Inflammatory skin diseases include, but are not limited to, conditions associated with cell proliferation, such as psoriasis, eczema and dermatitis, (e.g., eczematous dermatitides, topic and seborrheic dermatitis, allergic or irritant contact dermatitis, eczema craquelee, photoallergic dermatitis, phototoxic dermatitis, phytophotodermatitis, radiation dermatitis, and stasis dermatitis). Other inflammatory skin diseases include, but are not limited to, scleroderma, ulcers and erosions resulting from trauma, burns, bullous disorders, or ischemia of the skin or mucous membranes, several forms of ichthyoses, epidermolysis bullosae, hypertrophic scars, keloids, cutaneous changes of intrinsic aging, photoaging, frictional blistering caused by mechanical shearing of the skin and cutaneous atrophy resulting from the topical use of corticosteroids. Additional inflammatory skin conditions include inflammation of mucous membranes, such as cheilitis, chapped lips, nasal irritation, mucositis and vulvovaginitis.

Inflammatory disorders of the endocrine system include, but are not limited to, autoimmune thyroiditis (Hashimoto's disease), Type I diabetes, and acute and chronic inflammation of the adrenal cortex. Inflammatory conditions of the cardiovascular system include, but are not limited to, coronary infarct damage, peripheral vascular disease, myocarditis, vasculitis, revascularization of stenosis, artherosclerosis, and vascular disease associated with Type II diabetes.

Inflammatory condition of the kidney include, but are not limited to, glomerulonephritis, interstitial nephritis, lupus nephritis, nephritis secondary to Wegener's disease, acute renal failure secondary to acute nephritis, Goodpasture's syndrome, post-obstructive syndrome and tubular ischemia.

Inflammatory conditions of the liver include, but are not limited to, hepatitis (arising from viral infection, autoimmune responses, drug treatments, toxins, environmental agents, or as a secondary consequence of a primary disorder), biliary atresia, primary biliary cirrhosis and primary sclerosing cholangitis.

Inflammatory conditions of the central nervous system include, but are not limited to, multiple sclerosis and neurodegenerative diseases such as Alzheimer's disease, Parkinson's disease, or dementia associated with HIV infection.

Other inflammatory conditions include periodontal disease, tissue necrosis in chronic inflammation, endotoxin shock, smooth muscle proliferation disorders, graft versus host disease, tissue damage following ischemia reperfusion injury, and tissue rejection following transplant surgery.

The present invention further provides a method of treating or preventing inflammation associated with post-surgical wound healing in a patient comprising administering to said patient a compound of the invention.

It should be noted that compounds of the current invention may be used to treat or prevent any disease which has an inflammatory component, such as those diseases cited above. Further, the inflammatory conditions cited above are meant to be exemplary rather than exhaustive.

Those skilled in the art would recognize that additional inflammatory conditions (e.g., systemic or local immune imbalance or dysfunction due to an injury, an insult, infection, inherited disorder, or an environmental intoxicant or perturbant to the subject's physiology) may be treated or prevented by compounds of the current invention. Thus, the methods of the current invention may be used to treat or prevent any disease which has an inflammatory component, including, but not limited to, those diseases cited above.

The present invention also provides methods for treating or preventing arthritis, inflammatory bowel disease, uveitis, ocular inflammation, asthma, pulmonary inflammation, cystic fibrosis, psoriasis, arterial inflammation, cardiovascular diseases, multiple sclerosis, or neurodegenerative disease by administering an effective amount of a compound of the invention.

The present invention also provides methods for treating ischemia by administering an effective amount of a compound of the invention. In certain embodiments, the ischemia is cardiac ischemia, cerebral ischemia, bowel ischemia (e.g., ischemic colitis or mesenteric ischemia), or cutaneous ischemia.

The present invention provides a method of treating or preventing inflammatory disease in a patient comprising administering to said patient a compound of the invention conjointly with a glucocorticoid.

The present invention provides a method of treating or preventing an inflammatory condition (e.g., any of the inflammatory conditions described above) comprising administering to said patient a compound of the invention conjointly with a glucocorticoid.

Compounds of the invention are capable of resolving inflammation. Glucocorticoids are also known for their role in treating inflammation. However, the full anti-inflammatory potential of glucocorticoids is often clinically constrained as a monotherapy due to the rate and severity of treatment-limiting adverse events that accompany high or prolonged dosing regimens. For example, the administration of glucocorticoids can result in side effects that mimic Cushing's disease. These side effects and others associated with glucocorticoid use include increased appetite and weight gain, deposits of fat in the chest, face, upper back, and stomach, water and salt retention leading to swelling and edema, high blood pressure, diabetes, slow healing of wounds, osteoporosis, cataracts, acne, muscle weakness, thinning of the skin, increased susceptibility to infection, stomach ulcers, increased sweating, mood swings, psychological problems such as depression, and adrenal suppression and crisis. Advantageously, treatment of inflammatory disease with a combination of a glucocorticoid and a compound of the invention enhances the anti-inflammatory properties of both classes of compounds while reducing the effects associated with high doses of glucocorticoids alone.

In methods of the invention, wherein a glucocorticoid is administered conjointly with a compound of the invention, the glucocorticoid may be chosen from any glucocorticoid known in the art. Glucocorticoids suitable for said conjoint administration include, but are not limited to, alclometasone, amcinonide, beclometasone, betamethasone, budesonide, ciclesonide, clobetasol, clobetasone, clocortolone, cloprednol, cortisone, cortivazol, deflazacort, desonide, desoximetasone, desoxycortone, dexamethasone, diflorasone, diflucortolone, difluprednate, fluclorolone, fludroxycortide, flumetasone, flunisolide, fluocinolone acetonide, fluocinonide, fluocortin, fluocortolone, fluorometholone, fluperolone, fluprednidene, fluticasone, formocortal, halcinonide, halometasone, hydrocortisone/cortisol, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone/prednisolone, prednylidene, rimexolone, tixocortol, triamcinolone, ulobetasol, mometasone, fluticasone propionate, beclomethasone dipropionate, fluocinolone, flunisolide hemihydrate, mometasone furoate monohydrate, desoxymethasone, diflorasone diacetate, hydrocortisone acetate, difluorocortolone, fluorocortisone, flumethasone, flunisolide, fluorocortolone, prednisolone, prednisone, cortisol, 6a-methylprednisolone, alclometasone dipropionate, fluclorolone acetonide, fluocinolone acetonide, betamethasone benzoate, fluocoritin butyl, betamethasone dipropionate, fluocortolone preparations, betamethasone valerate, fluprednidene acetate, flurandrenolone, clobetasol propionate, clobetasol butyrate, hydrocortisone, hydrocortisone butyrate, methylprednisolone acetate, diflucortolone valerate, flumethasone pivalate, or triamcinolone acetonide, or pharmaceutically acceptable salts thereof.

In certain embodiments, the patient to be treated by a method of the invention may already be receiving an anti-inflammatory drug (other than a glucocorticoid). In one preferred embodiment, the patient is already taking a glucocorticoid, such as one of the glucocorticoids described above, and will continue to take that drug conjointly with a compound of the invention. Alternatively, the compound of the invention may be used as a replacement for the previously administered anti-inflammatory drug.

In a related embodiment, the invention provides a method of reducing the dose of a glucocorticoid required to achieve a desired anti-inflammatory effect. Reducing the dose of glucocorticoid while maintaining potent anti-inflammatory properties is highly desirable due to side effects associated with certain glucocorticoids. Side effects of glucocorticoids include increased appetite and weight gain, deposits of fat in the chest, face, upper back, and stomach, water and salt retention leading to swelling and edema, high blood pressure, diabetes, slow healing of wounds, osteoporosis, cataracts, acne, muscle weakness, thinning of the skin, increased susceptibility to infection, stomach ulcers, increased sweating, mood swings, psychological problems such as depression, and adrenal suppression and crisis.

In this embodiment, the dose of a glucocorticoid is reduced by at least 5%, at least 10%, at least 15%, at least 20%, at least 25%, at least 30%, at least 40%, at least 50%, at least 60%, at least 70%, at least 80%, at lest 90%, or more. The actual reduction in glucocorticoid dose will depend upon the nature and amount of the compound of the invention being administered, the reduction in inflammation desired, and other factors set forth elsewhere in this application that are typically considered in treating a disease or condition. The amount of the compound of the invention administered in this method will also depend upon the factors set forth above, as well as the nature and amount of glucocorticoid being administered. In certain embodiments, the amount of the compound of the invention administered in this method is less than 5%, less than 10%, less than 15%, less than 20%, less than 25%, less than 30%, less than 40%, less than 50%, less than 60%, less than 70%, less than 80%, or less than 90% of the dose of the compound of the invention required to produce an anti-inflammatory effect without conjoint administration with a glucocorticoid.

In one embodiment, the method of treating or preventing inflammatory disease according to this invention may comprise the additional step of conjointly administering to the patient another anti-inflammatory agent, such as, for example, a non-steroidal anti-inflammatory drug (NSAID), a mast cell stabilizer, or a leukotriene modifier.

In certain embodiments, the use of a composition comprising a compound of the invention and a glucocorticoid according to this invention in the treatment of inflammatory disease, does not preclude the separate but conjoint administration of another corticosteroid.

In certain embodiments, the use of a composition comprising both a compound of the invention and a glucocorticoid according to this invention in the treatment of inflammatory disease, does not preclude the separate but conjoint administration of another glucocorticoid.

In certain embodiments, different compounds of the invention may be conjointly administered with one or more other compounds of the invention while conjointly administering a glucocorticoid. Moreover, such combinations may be conjointly administered with other therapeutic agents, such as other anti-inflammatory agents.

In certain embodiments, the present invention provides a kit comprising: a) one or more single dosage forms of a compound of the invention; b) one or more single dosage forms of a glucocorticoid as mentioned above; and c) instructions for the administration of the compound of the invention and the glucocorticoid.

The present invention provides a kit comprising:
a) a pharmaceutical formulation (e.g., one or more single dosage forms) comprising a compound of the invention; and
b) instructions for the administration of the pharmaceutical formulation e.g., for treating or preventing a disorder or condition as discussed above, e.g., inflammatory disease.

In certain embodiments, the kit further comprises instructions for the administration of the pharmaceutical formulation comprising a compound of the invention conjointly with a glucocorticoid as mentioned above. In certain embodiments, the kit further comprises a second pharmaceutical formulation (e.g., as one or more single dosage forms) comprising a glucocorticoidas mentioned above.

In certain embodiments, the kit further comprises instructions for the administration of the pharmaceutical formulation comprising a compound of the invention conjointly with an agent suitable for the treatment or prevention of inflammatory disease (e.g., a non-steroidal anti-inflammatory drug (NSAID), a mast cell stabilizer, or a leukotriene modifier) as mentioned above. In certain embodiments, the kit further comprises a second pharmaceutical formulation (e.g., as one or more single dosage forms) comprising an agent suitable for the treatment or prevention of inflammatory disease (e.g., a non-steroidal anti-inflammatory drug (NSAID), a mast cell stabilizer, or a leukotriene modifier) as mentioned above.

The present invention provides a kit comprising:
a) a first pharmaceutical formulation (e.g., one or more single dosage forms) comprising an agent suitable for the treatment or prevention of inflammatory disease (e.g., a non-steroidal anti-inflammatory drug (NSAID), a mast cell stabilizer, or a leukotriene modifier) as mentioned above; and
b) instructions for the administration of the first pharmaceutical formulation with a compound of the invention, e.g., for treating or preventing inflammatory disease.

The present invention provides a kit comprising:
a) a first pharmaceutical formulation (e.g., one or more single dosage forms) comprising a glucocorticoid as mentioned above; and
b) instructions for the administration of the first pharmaceutical formulation with a compound of the invention, e.g., for treating or preventing inflammatory disease.

### Metabolic Diseases

The present invention provides a method of treating or preventing complex disorders having an inflammatory component in a patient comprising administering to said patient a compound of the invention. In certain embodiments, the complex disorder having an inflammatory component is type 2 diabetes or obesity.

The present invention provides a method of treating or preventing metabolic disorders in a patient comprising administering to said patient a compound of the invention. In certain embodiments, the metabolic disorder is selected from an eating disorder, dyslipidemia, hypertrigylceridemia, hypertension, or metabolic syndrome.

The present invention further provides a method of treating or preventing type 1 diabetes in a patient, comprising administering to said patient a compound of the invention. In certain embodiments, the present invention provides a method of treating a patient at risk of developing type 1 diabetes comprising administering to said patient a compound of the invention. In certain embodiments, the present invention provides a method of treating a patient exhibiting one or more warning signs of type 1 diabetes, such as extreme thirst; frequent urination; drowsiness or lethargy; sugar in urine; sudden vision changes; increased appetite; sudden weight loss; fruity, sweet, or wine-like odor on breath; heavy, labored breathing; stupor; or unconsciousness, comprising administering to said patient a compound of the invention.

The present invention further provides a method of treating or preventing type 2 diabetes in a patient, comprising administering to said patient a compound of the invention. In certain embodiments, the present invention provides a method of treating a patient at risk of developing type 2 diabetes comprising administering to said patient a compound of the invention. In certain embodiments, the present invention provides a method of treating a patient exhibiting one or more warning signs of type 2 diabetes, such as extreme thirst; frequent urination; drowsiness or lethargy; sugar in urine; sudden vision changes; increased appetite; sudden weight loss; fruity, sweet, or wine-like odor on breath; heavy, labored breathing; stupor; or unconsciousness, comprising administering to said patient a compound of the invention.

The present invention further provides a method for protecting, e.g., promoting the growth and/or survival of, beta cells of Islets of Langerhans from lipid- or glucose-triggered toxicity in a patient comprising administering to the patient a compound of the invention.

In certain embodiments, the methods of treating or preventing a complex disorder having an inflammatory component, such as type 2 diabetes, or of treating type 1 diabetes according to this invention may comprise the additional step of conjointly administering to the patient another treatment for diabetes including, but not limited to, sulfonylureas (e.g., chlorpropamide, tolbutamide, glyburide, glipizide, acetohexamide, tolazamide, gliclazide, gliquidone, or glimepiride), medications that decrease the amount of glucose produced by the liver (e.g., metformin), meglitinides (e.g., repaglinide or nateglinide), medications that decrease the absorption of carbohydrates from the intestine (e.g., alpha glucosidase inhibitors such as acarbose), medications that effect glycemic control (e.g., pramlintide or exenatide), DPP-IV inhibitors (e.g., sitagliptin), insulin treatment, or combinations of the above.

In certain embodiments, the methods of treating or preventing a complex disorder having an inflammatory component, such as obesity, according to this invention may comprise the additional step of conjointly administering to the patient another treatment for obesity including, but not limited to, orlistat, sibutramine, phendimetrazine, phentermine, diethylpropion, benzphetamine, mazindol, dextroamphetamine, rimonabant, cetilistat, GT 389-255, APD356, pramlintide/AC137, PYY3-36, AC 162352/PYY3-36, oxyntomodulin, TM 30338, AOD 9604, oleoyl-estrone, bromocriptine, ephedrine, leptin, pseudoephedrine, or pharmaceutically acceptable salts thereof.

In certain embodiments, different compounds of the invention may be conjointly administered with one or more other compounds of the invention. Moreover, such combinations may be conjointly administered with other therapeutic agents, such as other agents suitable for the treatment or prevention of metabolic disorders, such as the agents identified above.

The present invention provides a kit comprising:
a) a pharmaceutical formulation (e.g., one or more single dosage forms) comprising a compound of the invention; and
b) instructions for the administration of the pharmaceutical formulation e.g., for treating or preventing complex disorders having an inflammatory component (e.g., type 2 diabetes or obesity), treating or preventing metabolic disorders (e.g., eating disorder, dyslipidemia, hypertrigylceridemia, hypertension, or metabolic syndrome), treating or preventing type 1 diabetes, treating a patient at risk of developing type 1 diabetes, treating a patient exhibiting warning signs of type 1 diabetes, or protecting (e.g., promoting the growth and/or survival of) beta cells of Islets of Langerhans from lipid- or glucose-triggered toxicity.

In certain embodiments, the kit further comprises instructions for the administration of the pharmaceutical formulation comprising a compound of the invention conjointly with an agent suitable for the treatment or prevention of diabetes (e.g., sulfonylureas (e.g., chlorpropamide, tolbutamide, glyburide, glipizide, acetohexamide, tolazamide, gliclazide, gliquidone, or glimepiride), medications that decrease the amount of glucose produced by the liver (e.g., metformin), meglitinides (e.g., repaglinide or nateglinide), medications that decrease the absorption of carbohydrates from the intestine (e.g., alpha glucosidase inhibitors such as acarbose), medications that effect glycemic control (e.g., pramlintide or exenatide), DPP-IV inhibitors (e.g., sitagliptin), insulin treatment, or combinations of the above) as mentioned above. In certain embodiments, the kit further comprises a second pharmaceutical formulation (e.g., as one or more single dosage forms) comprising an agent suitable for the treatment or prevention of diabetes (e.g., sulfonylureas (e.g., chlorpropamide, tolbutamide, glyburide, glipizide, acetohexamide, tolazamide, gliclazide, gliquidone, or glimepiride), medications that decrease the amount of glucose produced by the liver (e.g., metformin), meglitinides (e.g., repaglinide or nateglinide), medications that decrease the absorption of carbohydrates from the intestine (e.g., alpha glucosidase inhibitors such as acarbose), medications that effect glycemic control (e.g., pramlintide or exenatide), DPP-IV inhibitors (e.g., sitagliptin), insulin treatment, or combinations of the above) as mentioned above.

In certain embodiments, the kit further comprises instructions for the administration of the pharmaceutical formulation comprising a compound of the invention conjointly with an agent suitable for the treatment or prevention of obesity, as mentioned above. In certain embodiments, the kit further comprises a second pharmaceutical formulation (e.g., as one or more single dosage forms) comprising an agent suitable for the treatment or prevention of obesity, as mentioned above.

The present invention provides a kit comprising:
a) a first pharmaceutical formulation (e.g., one or more single dosage forms) comprising an agent suitable for the treatment or prevention of obesity as mentioned above; and
b) instructions for the administration of the first pharmaceutical formulation with a compound of the invention, e.g., for treating or preventing complex disorders having an inflammatory component (e.g., type 2 diabetes or obesity), treating or preventing metabolic disorders (e.g., eating disorder, dyslipidemia, hypertrigylceridemia, hypertension, or metabolic syndrome), treating or preventing type 1 diabetes, treating a patient at risk of developing type 1 diabetes, treating a patient exhibiting warning signs of type 1 diabetes, or protecting (e.g., promoting the growth and/or survival of) beta cells of Islets of Langerhans from lipid- or glucose-triggered toxicity.

The present invention provides a kit comprising:
a) a first pharmaceutical formulation (e.g., one or more single dosage forms) comprising an agent suitable for the treatment or prevention of diabetes as mentioned above; and
b) instructions for the administration of the first pharmaceutical formulation with a compound of the invention, e.g., for treating or preventing complex disorders having an inflammatory component (e.g., type 2 diabetes or obesity), treating or preventing metabolic disorders (e.g., eating disorder, dyslipidemia, hypertrigylceridemia, hypertension, or metabolic syndrome), treating or preventing type 1 diabetes, treating a patient at risk of developing type 1 diabetes, treating a patient exhibiting warning signs of type 1 diabetes, or protecting (e.g., promoting the growth and/or survival of) beta cells of Islets of Langerhans from lipid- or glucose-triggered toxicity.

Ophthalmic Conditions

The present invention provides a method of treating or preventing an ophthalmic condition in a patient, comprising administering to said patient a compound of the invention.

Examples of ophthalmic conditions that may be treated by administration of a compound of the invention, include, but are not limited to, AIDS-related retinal disorders; age-related macular degeneration; alkaline erosive keratoconjunctivitis; allergic keratitis; anterior ischemic optic neuropathy; anterior uveitis (iridocyclitis); Behcet's disease; blepharitis; seborrheic blepharitis; canaliculitis; cataract; central serous chorioretinopathy; chorioiditis; chronic uveitis; Coats' disease; conjunctivitis (e.g., infectious conjunctivitis, neonatal conjunctivitis, non-infectious conjunctivitis, and allergic conjunctivitis); contact lens-induced keratoconjunctivitis; contact eczema; corneal ulcer (e.g., Mooren's ulcer, corneal ulcer subsequent to chronic rheumatoid arthritis or collagen disease, Terrien's marginal degeneration, catarrhal corneal ulcer, infectious corneal ulcer); crystalline retinopathy; cyclitis; edema (e.g., cystoid macular edema); dacryoadenitis; dacryocystitis; degenerative myopia; degenerative retinoschisis; diabetic keratophathy; diabetic macular edema; diabetic retinopathy; dry eye disease (e.g., dry eye of the lacrimal system or dry eye of the cornea); dry age-related macular degeneration; endophthalmitis; episcleritis; exudative macular edema; Fuchs' Dystrophy; giant cell arteritis; giant papillary conjunctivitis; glaucoma (e.g., primary open angle glaucoma, primary angle closure glaucoma, secondary open angle glaucoma, secondary angle closure glaucoma, and childhood glaucoma); glaucoma surgery failure; graft versus host disease of the eye (often a form of dry eye); herpes zoster (shingles); hypertensive retinopathy; inflammation after cataract surgery; iridocorneal endothelial syndrome; iridocytis; iritis; keratitis (e.g., infectious keratitis, non-infectious keratitis, and neuroparalytic keratitis); keratoconjunctiva sicca; keratoconjunctival inflammatory disease; keratoconus; keratopathy; lattice dystrophy; map-dot-fingerprint dystrophy; necrotic keratitis; neovascular diseases involving the retina, uveal tract or cornea such as neovascular glaucoma, corneal neovascularization (inflammatory, transplantation, developmental hypoplasia of the iris), neovascularization resulting following a combined vitrectomy and lensectomy, neovascularization of the optic nerve, and neovascularization due to penetration of the eye or contusive ocular injury; non-infectious uveitis; ocular herpes; ocular rosacea; ophthalmic infections (e.g., corneal herpes, bacterial keratitis, bacterial conjunctivitis, mycotic keratitis, acanthamebic keratitis, infectious endophthalmitis, infectious corneal ulcer, inflammation of the conjunctiva or cornea by staphylococci, streptococci, enterococci, euterococci, bacillus, corynebacterium, chlamydia, and neisseria); ophthalmic pemphigoid; optic disc drusen; optic neuritis; panuveitis; papilledema; papillitis; pars planitis; persistent macular edema; phacoanaphylaxis; posterior uveitis (chorioentinitis); post-operative inflammation (e.g., post-LASIK inflammation of the cornea); proliferative diabetic retinopathy; proliferative sickle cell retinopathy; proliferative vitreoretinopathy; retinal artery occlusion; retinal detachment; retinal vasculitis; retinal vein occlusion; retinitis pigmentosa; retinopathy of prematurity; rubeosis iritis; scleritis; Stevens-Johnson syndrome (erythema multiforme major); sympathetic ophthalmia; temporal arteritis; toxic retinopathy; uveitis (e.g., anterior uveitis or posterior uveitis); vernal conjunctivitis; vitamin A insufficiency-induced keratomalacia; vitreitis; and wet age-related macular degeneration.

Diseases causing dry eye include Riley-Day syndrome, Shy-Drager syndrome, Sjogren syndrome, sarcoidosis, amyloidosis, sequela of radiotherapy, lagophthalmia, avitaminosis A, Stevens-Johnson syndrome, ocular pemphigoid, marginal blepharitis, meibomitis, sequela of intraocular surgery, contact-lens affection, diabetic corneal epitheliopathy, dry eye due to VDT operation, graft versus host disease, and the like. Disorders caused by corneal infective disease include, for example, viral epitheliopathy and the like. Stem cell depletion syndromes include Stevens-Johnson syndrome, ocular pemphigoid, thermal or chemical burn, drug toxicity of idoxuridine (IDU) and therapeutic agents for glaucoma, and the like.

The present invention provides a method of inhibiting COX-2 or TNF in the eye in a patient comprising administering to said patient a compound of the invention. The present invention further provides a method of protecting against goblet cell loss in the eye in a patient comprising administering to said patient a compound of the invention.

Compounds as described herein may also inhibit inflammatory mediators in the cornea, such as TNF, IL-1a, IL-1b, IL-6, and IL-8. Accordingly, these compounds may be useful in the treatment of dry eyes diseases, age-related macular degeneration, retinopathy of prematurity, uveitis, and glaucoma.

Compounds as described herein may also inhibit COX-2 in the cornea. Accordingly, these compounds may be useful in the treatment of dry eyes diseases.

Compounds as described herein may also guard against goblet cell loss. Accordingly, these compounds may be useful in the treatment of dry eye diseases, age-related macular degeneration, retinopathy of prematurity, retinitis pigmentosa, and glaucoma. Compounds as described herein may also induce increases in tear production and density of superficial epithelial cells, two endpoints relevant to the treatment of dry eye.

Compounds as described herein may also reduce vascular leakage. Accordingly, these compounds may be useful in the treatment of age-related macular degeneration.

Compounds as described herein may also inhibit CD11b+ cells. Animal models of dry eye show an increase in CD11b+ cells suggesting the increased presence of leukocytes in corneas. Accordingly, these compounds may be useful in the treatment of dry eye by decreasing the arrival of leukocytes induced by dry eye.

Compounds as described herein may also prevent pigmented retinal epithelium destruction. Accordingly, these compounds may be useful in the treatment of age-related macular degeneration, retinopathy of prematurity, retinitis pigmentosa, and glaucoma.

In certain embodiments, different compounds of the invention may be conjointly administered with other agents suitable for the treatment or prevention of an ophthalmic condition. For example, the following agents or classes of agents may be conjointly administered with a compound of the invention: doxocycline; decosahexanoic acid; angiogenesis inhibitors, e.g., VEGF inhibitors, such as pegaptanib sodium, bevacizumab, ranibizumab, AV-951, vandetanib, semaxanib, CBO-P11, axitinib, sorafenib, sunitinib, pazopanib, and TIMP3; anesthetics and pain killing agents such as lidocaine and related compounds and benzodiazepam and related compounds; anti-cancer agents such as 5-fluorouracil, adriamycin and related compounds; anti-inflammatory agents such as 6-mannose phosphate; anti-fungal agents such as fluconazole and related compounds; anti-viral agents such as trisodium phosphomonoformate, trifluorothymidine, acyclovir, ganciclovir, DDI, DDC, and AZT; cell transport/mobility impending agents such as colchicine, vincristine, cytochalasin B, and related compounds; antiglaucoma drugs such as beta-blockers: timolol, betaxol, atenalol, etc; prostaglandins such as latanoprost and travoprost, etc.; immunological response modifiers such as muramyl dipeptide and related compounds; peptides and proteins such as cyclosporin, insulin, growth hormones, insulin related growth factor, nerve growth factor (optionally in further combination with decosahexanoic acid), heat shock proteins and related compounds; estrogen treatments; corticosteroids such as dexamethasone, dexamethasone 21-phosphate, fluorometholone, medrysone, betamethasone, triamcinolone, triamcinolone acetonide, triminolone, prednisone, prednisolone, prednisolone 21-phosphate, prednisolone acetate, hydrocortisone, hydrocortisone acetate, prednicarbate, deflazacort, halomethasone, tixocortol, prednylidene (21-diethylaminoacetate), prednival, paramethasone, prednisolone, methylprednisolone, meprednisone, mazipredone, isoflupredone, halopredone acetate, halcinonide, formocortal, flurandrenolide, fluprednisolone, flurprednidine acetate, fluperolone acetate, fluocortolone, fluocortin butyl, fluocinonide, fluocinolone, fluocinolone acetonide, flunisolide, flumethasone, fludrocortisone, fluclorinide, fluoromethalone, enoxolone, difluprednate, diflucortolone, diflorasone diacetate, desoximetasone (desoxymethasone), desonide, descinolone, cortivazol, corticosterone, cortisone, cloprednol, clocortolone, clobetasone, clobetasol, chloroprednisone, cafestol, budesonide, beclomethasone, amcinonide, allopregnane acetonide, alclometasone, 21-acetoxypregnenolone, tralonide, diflorasone acetate, deacylcortivazol, RU-26988, budesonide, and deacylcortivazol oxetanone. All of the above-cited corticosteroids are known compounds. Further information about the compounds may be found, for example, in The Merck Index, Thirteenth Edition (2001), and the publications cited therein, the entire contents of which are hereby incorporated herein by reference. In certain embodiments, the corticosteroid is selected from fluocinolone acetonide, triamcinolone acetonide, dexamethasone, and related compounds, or any combination thereof; and carbonic anhydaze inhibitors.

Further examples of agents or classes of agents may be conjointly administered with a compound of the invention include: DE-104; PF-04217329; PF-03187207; AL 37807; OPC-12759; chemotherapeutic agents such as mitomycin C; synthetic structural analogs of prostaglandin such as bimatoprost; alpha 2 agonists such as brimonidine; carbonic anhydrase inhibitors such as dorzolamide HCl; prostaglandin derivatives and analogs such as tafluprost and travoprost; NMDA antagonists such as memantine; hyaluronic acid (e.g., sodium hyaluronate); corticosteroids such as loteprednol etabonate, difluprednate and rimexolone; antibiotics such as doxycycline; agents that increase mucin such as ecabet and rebamipide; lubricants such as the combination of carboxymethylcellulose sodium and glycerin; A3 adenosine receptor agonists such as CF-101; immunomodulators such as thalidomide; TNFα antagonists such as etanercept; protein kinase C-b inhibitors such as ruboxistaurin; immunosuppressants such as sirolimus; PARP inhibitors such as AG-014699; neuroprotective thrombolytic agents such as microplasmin; hyaluronidase; oxidizing agents such as carbamide; somatostatin analogs such as octreotide acetate; angiotensin II receptor antagonists such as candesartan cilexetil; disease-modifying antirheumatic drugs such as leflunomide; AEB071; TNF antagonists such as adalimumab; CD11 antagonists such as efalizumab; calcineurin inhibitors such as LX211; interferons such as interferon α-2a; and human alpha fetoproteins such as MM-093.

In addition to the above agents, other agents are suitable for administration to the eye and its surrounding tissues to produce a local or a systemic physiologic or pharmacologic beneficial effect. Such agents may be conjointly administered with a compound of the invention. Examples of such agents include neuroprotectants, such as nimodipine and related compounds; antibiotics, such as tetracycline, chlortetracycline, bacitracin, neomycin, polymyxin, gramicidin, oxytetracycline, chloramphenicol, gentamycin, and erythromycin; antibacterials, such as sulfonamides, sulfacetamide, sulfamethizole, and sulfisoxazole; antivirals, such as, idoxuridine; other antibacterial agents, such as nitrofurazone and sodium propionate; antiallergenics, such as antazoline, methapyriline, chlorpheniramine, pyrilamine, and prophenpyridamine; decongestants, such as phenylephrine, naphazoline, and tetrahydrazoline; miotics and anti-cholinesterase, such as pilocarpine, eserine salicylate, carbachol, di-isopropyl fluorophosphate, phospholine iodine, and demecarium bromide; mydriatics, such as atropine sulfate, cyclopentolate, homatropine, scopolamine, tropicamide, eucatropine, and hydroxyamphetamine; sympathomimetics, such as epinephrine; and prodrugs, such as those described in Design of Prodrugs, edited by Hans Bundgaard, Elsevier Scientific Publishing Co., Amsterdam, 1985. Reference may be made to any standard pharmaceutical textbook such as Remington's Pharmaceutical Sciences (Remington's Pharmaceutical Sciences. Mack Publishing Company, Easton, Pa., USA 1985) for the identity of other agents.

In certain embodiments, different compounds of the invention may be conjointly administered with non-chemical methods suitable for the treatment or prevention of an ophthalmic condition. In certain embodiments, different compounds of the invention may be conjointly administered with laser treatment (e.g., photocoagulation or photodynamic therapy), macular translocation surgery or with devices (e.g., brimonidine tartrate implant).

In certain embodiments, different compounds of the invention may be conjointly administered with one or more other compounds of the invention. Moreover, such combinations may be conjointly administered with other therapeutic agents, such as other agents suitable for the treatment or prevention of an ophthalmic condition, such as the agents identified above.

In certain embodiments, the present invention provides a kit comprising: a) one or more single dosage forms of a compound of the invention; b) one or more single dosage forms of an agent suitable for the treatment or prevention of an ophthalmic condition or an agent suitable for administration to the eye and its surrounding tissues to produce a local or a systemic physiologic or pharmacologic beneficial effect as mentioned above; and c) instructions for the administration of the compound of the invention and the agent suitable for the treatment or prevention of an ophthalmic condition or agent suitable for administration to the eye and its surrounding tissues to produce a local or a systemic physiologic or pharmacologic beneficial effect.

The present invention provides a kit comprising:
a) a pharmaceutical formulation (e.g., one or more single dosage forms) comprising a compound of the invention; and
b) instructions for the administration of the pharmaceutical formulation e.g., for treating or preventing an ophthalmic condition, inhibiting COX-2 or TNF in the eye, or protecting against goblet cell loss in the eye.

In certain embodiments, the kit further comprises instructions for the administration of the pharmaceutical formulation comprising a compound of the invention conjointly with an agent suitable for the treatment or prevention of an ophthalmic condition, an agent suitable for administration to the eye and its surrounding tissues to produce a local or a systemic physiologic or pharmacologic beneficial effect, or non-chemical methods suitable for the treatment or prevention of an ophthalmic condition as mentioned above. In certain embodiments, the kit further comprises a second pharmaceutical formulation (e.g., as one or more single dosage forms) comprising an agent suitable for the treatment or prevention of an ophthalmic condition or an agent suitable for administration to the eye and its surrounding tissues to produce a local or a systemic physiologic or pharmacologic beneficial effect as mentioned above.

The present invention provides a kit comprising:
a) a first pharmaceutical formulation (e.g., one or more single dosage forms) comprising an agent suitable for the treatment or prevention of an ophthalmic condition or an agent suitable for administration to the eye and its surrounding tissues to produce a local or a systemic physiologic or pharmacologic beneficial effect as mentioned above; and
b) instructions for the administration of the first pharmaceutical formulation with a compound of the invention, e.g., for treating or preventing an ophthalmic condition, inhibiting COX-2 or TNF in the eye, or protecting against goblet cell loss in the eye.

### Immune Function

The present invention provides a method of inhibiting immune function and/or suppressing an immune response in a patient, comprising administering to said patient a compound of the invention.

The present invention provides a method of treating or preventing an autoimmune disease or an autoimmune disorder in a patient, comprising administering to said patient a compound of the invention.

In certain embodiments, the autoimmune disease or autoimmune disorder is of the type where the patient's own immune system damages one or more of the patient's tissues. In certain embodiments, the autoimmune disease or autoimmune disorder may be triggered by something within the patient or something within the patient's environment.

In certain embodiments, the autoimmune disease or autoimmune disorder of the present invention may be one which follows an initiating cause. For example, the autoimmune disease or autoimmune disorder may be one which is caused by an infection and/or some other initiating cause. Potential initiating causes may include old age, infection (such as parasitic infection), treatment with steroids, repeated vaccination with alum, pregnancy and/or cancers.

In certain embodiments, the autoimmune disease or autoimmune disorder may be organ-specific or non-organ-specific. Examples of such autoimmune diseases or autoimmune disorders include multiple sclerosis, arthritis (e.g., rheumatoid arthritis or juvenile arthritis), Crohn's disease, colitis ulcerosa and aplastic anemia systemic lupus erythematosus (SLE or lupus), dermatomyositis, pernicious anemia, Addison's disease, ankylosing spondylitis, antiphospholipid syndrome, Churg-Strauss Syndrome, discoid lupus, fibromyalgia, Grave's Disease, myasthenia gravis, psoriasis, Reiter's Syndrome, rheumatic fever, sarcoidosis, scleroderma, Sjogren's Syndrome, stiff-man syndrome, thyroiditis, uveitis, vitiligo, Wegener's granulomatosis, graft rejection, and vascular disorders.

In certain embodiments wherein the autoimmune disease or autoimmune disorder is graft rejection, the graft rejection may be chronic graft rejection. In certain embodiments of the present invention wherein a compound of the invention is administered for the treatment of graft rejection, the administration of a compound of the invention modulates immune responses to grafts (e.g., allografts or xenografts) where untreated rejection would otherwise lead to graft loss. Thus, a compound of the invention may be used as a replacement for or in addition to the conventional immunosuppressant administered prior to, during and/or after transplantation. In certain embodiments, the graft rejection is in response to transplanting natural or artificial cells, islet cells, tissues (e.g., natural or artificial skin tissue), corneas, bone marrow, organs (e.g. kidney, liver, pancreas, lung, or heart), lenses, or pacemakers.

The present invention further provides a method of treating or preventing a disease, sequela or pathological condition mediated by an activation of the immune system in a patient, comprising administering to said patient a compound of the invention. In certain embodiments, diseases, sequelae and pathological conditions mediated by an activation of the immune system include, but are not limited to, capillary leakage, pulmonary failure, sepsis, endotoxic shock, or sequelae of tissue damage.

In certain embodiments, different compounds of the invention may be conjointly administered with other agents suitable for modulating immune function, suppressing immune response, treating an autoimmune disease or autoimmune disorder, or treating a disease, sequela or pathological condition mediated by an activation of the imune system. For example, the following immunosuppressive agents may be conjointly administered with a compound of the invention: cyclosporin, cyclosporin A, tacrolimus, rapamycin, everolimus, FK-506, cyclophosphamide, azathioprene, methotrexate, brequinar, leflunomide, mizoribine, mycophenolic acid, mycophenolate mofetil, 15-deoxyspergualine, triamcinolone acetonide, decadron, daclizumab, basiliximab, glatiramer acetate, infliximab, muromonab, octreotide, muramylic acid dipeptide derivatives, levamisole, niridazole, oxysuran, flagyl, and sirolimus.

In certain embodiments, different compounds of the invention may be conjointly administered with one or more other compounds of the invention. Moreover, such combinations may be conjointly administered with other therapeutic agents, such as other agents suitable for modulating immune function, suppressing immune response, treating or preventing an autoimmune disease or autoimmune disorder, or treating or preventing a disease, sequela or pathological condition mediated by an activation of the imune system, such as the agents identified above.

In certain embodiments, the present invention provides a kit comprising: a) one or more single dosage forms of a compound of the invention; b) one or more single dosage forms of an agent suitable for modulating immune function, suppressing immune response, treating or preventing an autoimmune disease or autoimmune disorder, or treating or preventing a disease, sequela or pathological condition mediated by an activation of the imune system as mentioned above; and c) instructions for the administration of the compound of the invention and the agent suitable for modulating immune function, suppressing immune response, treating or preventing an autoimmune disease or autoimmune disorder, or treating or preventing a disease, sequela or pathological condition mediated by an activation of the imune system.

The present invention provides a kit comprising:
a) a pharmaceutical formulation (e.g., one or more single dosage forms) comprising a compound of the invention; and
b) instructions for the administration of the pharmaceutical formulation, e.g., for inhibiting immune function, suppressing an immune response, treating or preventing an autoimmune disease or an autoimmune disorder, or treating or preventing a disease, sequela or pathological condition mediated by an activation of the immune system.

In certain embodiments, the kit further comprises instructions for the administration of the pharmaceutical formulation comprising a compound of the invention conjointly with an agent suitable for modulating immune function, suppressing immune response, treating or preventing an autoimmune disease or autoimmune disorder, or treating or preventing a disease, sequela or pathological condition mediated by an activation of the imune system as mentioned above. In certain embodiments, the kit further comprises a second pharmaceutical formulation (e.g., as one or more single dosage forms) comprising an agent suitable for modulating immune function, suppressing immune response, treating or preventing an autoimmune disease or autoimmune disorder, or treating or preventing a disease, sequela or pathological condition mediated by an activation of the imune system as mentioned above.

The present invention provides a kit comprising:
a) a first pharmaceutical formulation (e.g., one or more single dosage forms) comprising an agent suitable for modulating immune function, suppressing immune response, treating or preventing an autoimmune disease or autoimmune disorder, or treating or preventing a disease, sequela or pathological condition mediated by an activation of the imune system as mentioned above; and
b) instructions for the administration of the first pharmaceutical formulation with a compound of the invention, e.g., for inhibiting immune function, suppressing an immune response, treating or preventing an autoimmune disease or an autoimmune disorder, or treating or preventing a disease, sequela or pathological condition mediated by an activation of the immune system.

Pulmonary Conditions

The present invention provides a method of treating or preventing a pulmonary condition in a patient comprising administering a compound of the invention. In certain embodiments, the pulmonary condition may be selected from pulmonary inflammation, airway inflammation, asthma, chronic bronchitis, bronchiectasis, non-cystic fibrosis-related bronchiectasis, cystic fibrosis, eosinophilic lung diseases (e.g., parasitic infection, idiopathic eosinophilic pneumonias, and Churg-Strauss vasculitis), allergic bronchopulmonary aspergillosis, allergic inflammation of the respiratory tract (e.g., rhinitis and sinusitis), bronchiolitis, bronchiolitis obliterans, bronchiolitis obliterans with organizing pneumonia, eosinophilic granuloma, Wegener's granulomatosis, sarcoidosis, hypersensitivity pneumonitis, idiopathic pulmonary fibrosis, pulmonary manifestations of connective tissue diseases, acute or chorionic lung injury, adult respiratory distress syndrome, pneumonia, emphysema, pulmonary disorders, or chronic obstructive pulmonary disease.

In certain embodiments, different compounds of the invention may be conjointly administered with one or more other compounds of the invention. Moreover, such combinations may be conjointly administered with other therapeutic agents, such as other agents suitable for the treatment or prevention of a pulmonary condition, such as the conditions disclosed herein. Exemplary agents suitable for the treatment of pulmonary conditions include corticosteroids (e.g., prednisone, beclomethasone dipropionate, fluticasone propionate, and other suitable corticosteroids), beta-agonists (e.g., albuterol, metaproterenol, pirbuterol, formoterol, terbutaline, isoetharine, levalbuterol, salmeterol xinafoate, and other suitable beta-agonists), and anti-cholinergic agents (e.g., ipratropium bromide, tiotropium bromide, and other suitable anti-cholinergic agents).

In certain embodiments, the present invention provides a kit comprising: a) one or more single dosage forms of a compound of the invention; b) one or more single dosage forms of an agent suitable for the treatment or prevention of a pulmonary condition; and c) instructions for the administration of the compound of the invention and the agent suitable for the treatment or prevention of a pulmonary condition.

The present invention provides a kit comprising:
a) a pharmaceutical formulation (e.g., one or more single dosage forms) comprising a compound of the invention; and
b) instructions for the administration of the pharmaceutical formulation, e.g., for treating or preventing a condition as discussed above, e.g., a pulmonary condition.

In certain embodiments, the kit further comprises instructions for the administration of the pharmaceutical formulation comprising a compound of the invention conjointly with an agent suitable for the treatment or prevention of a pulmonary condition. In certain embodiments, the kit further comprises a second pharmaceutical formulation (e.g., as one or more single dosage forms) comprising an agent suitable for the treatment or prevention of a pulmonary condition.

In certain embodiments, the kit further comprises instructions for the administration of the one or more single dosage forms each comprising a compound of the invention conjointly with an agent suitable for the treatment or prevention of a pulmonary condition. In certain embodiments, the kit further comprises one or more single dosage forms of an agent suitable for the treatment or prevention of a pulmonary condition.

The present invention provides a kit comprising:
a) a first pharmaceutical formulation (e.g., one or more single dosage forms) comprising an agent suitable for the treatment or prevention of a pulmonary condition; and
b) instructions for the administration of the first pharmaceutical formulation with a compound of the invention, e.g., for treating or preventing a condition as discussed above, e.g., for treating or preventing a pulmonary condition.

### Gastrointestinal Conditions

The present invention provides a method of treating or preventing a gastrointestinal condition in a patient comprising administering a compound of the invention. In certain embodiments, the gastrointestinal condition may be selected from gastrointestinal inflammation, ulcerative colitis, clostridium difficile colitis, microscopic or lymphocytic colitis, collagenous colitis, Crohn's disease, irritable bowel syndrome, infectious enteritis, antibiotic associative diarrhea, colon polyps, familial polyps, familial polyposis syndrome, Gardner's syndrome, helicobacter pylori, nonspecific diarrheal illnesses, intestinal cancers, distal proctitis, inflammatory states associated with abnormal colonic muscular contraction (e.g., spastic colon and mucous colitis), allergic bowel disease (e.g., coeliac disease), esophogitis, or pancreatitis.

In certain embodiments, different compounds of the invention may be conjointly administered with one or more other compounds of the invention. Moreover, such combinations may be conjointly administered with other therapeutic agents, such as other agents suitable for the treatment or prevention of a gastrointestinal condition, such as the conditions disclosed herein. Exemplary agents suitable for the treatment or prevention of gastrointestinal conditions include immunosuppressive agents (e.g., corticosteroids), histamine-2 receptor antagonists (e.g., cimetidine, famotidine, nizatidine, and ranitidine), sucralfate, prostaglandins (e.g., misopostol), and proton pump inhibitors (e.g., omeprazole, lansoprazole, esomeprazole, pantoprazole, and rabeprazole).

In certain embodiments, the present invention provides a kit comprising: a) one or more single dosage forms of a compound of the invention; b) one or more single dosage forms of an agent suitable for the treatment or prevention of a gastrointestinal condition; and c) instructions for the administration of the compound of the invention and the agent suitable for the treatment or prevention of a gastrointestinal condition.

The present invention provides a kit comprising:
a) a pharmaceutical formulation (e.g., one or more single dosage forms) comprising a compound of the invention; and
b) instructions for the administration of the pharmaceutical formulation, e.g., for treating or preventing a condition as discussed above, e.g., a gastrointestinal condition.

In certain embodiments, the kit further comprises instructions for the administration of the pharmaceutical formulation comprising a compound of the invention conjointly with an agent suitable for the treatment or prevention of a gastrointestinal condition. In certain embodiments, the kit further comprises a second pharmaceutical formulation (e.g., as one or more single dosage forms) comprising an agent suitable for the treatment or prevention of a gastrointestinal condition.

The present invention provides a kit comprising:
a) a first pharmaceutical formulation (e.g., one or more single dosage forms) comprising an agent suitable for the treatment or prevention of a gastrointestinal condition; and
b) instructions for the administration of the first pharmaceutical formulation with a compound of the invention, e.g., for treating or preventing a condition as discussed above, e.g., a gastrointestinal condition.

### Rheumatological Conditions

The present invention provides a method of treating or preventing a rheumatological condition in a patient comprising administering a compound of the invention. In certain embodiments, the rheumatological condition may be selected from rheumatic diseases, rheumatoid arthritis, osteoarthritis, inflammatory conditions of joints (e.g., rheumatoid arthritis, rheumatoid spondylitis, osteoarthritis, gouty arthritis), ankylosing spondylitis, lupus, psoriatic arthritis, myalgias, or chronic low back pain.

In certain embodiments, different compounds of the invention may be conjointly administered with one or more other compounds of the invention. Moreover, such combinations may be conjointly administered with other therapeutic agents, such as other agents suitable for the treatment or prevention of a rheumatological condition, such as the conditions disclosed herein. Exemplary agents suitable for the treatment or prevention of rheumatological conditions include non-steroidal anti-inflammatory drugs such as salicylates (e.g., aspirin, amoxiprin, benorilate, choline magnesium salicylate, diflunisal, faislamine, methyl salicylate, magnesium salicylate, salicyl salicylate), arylalkanoic acids (e.g., diclofenac, aceclofenac, acemetacin, bromfenac, etodolac, indometacin, nabumetone, sulindac, and tolmetin), 2-arylpropionic acids (e.g., ibuprofen, carprofen, fenbufen, fenoprofen, flurbiprofen, ketoprofen, ketorolac, loxoprofen, naproxen, oxaprozin, tiaprofenic acid, and suprofen), N-arylanthranilic acids (e.g., mefenamic acid and meclofenamic acid), pyrazolidine derivatives (e.g., phenylbutazone, azapropazone, metamizole, oxyphenbutazone, and sulfinpyrazone), oxicams (e.g., piroxicam, lornoxicam, meloxicam, and tenoxicam), COX-2 inhibitors (e.g., celecoxib, etoricoxib, lumiracoxib, parecoxib, rofecoxib, and valdecoxib) and sulphonanilides (e.g., nimesulide), corticosteroids (e.g., prednisone, cortisone, solumedrol, and hydrocortisone), disease-modifying anti-rheumatic drugs (e.g., leflunomide, oral gold, sulfasalazine, mycophenolate, cyclophosphamide, azathioprine, chlorambucil, rheumatrex, minocycline, gold shots, cuprimine, and quineprox), pain medication (e.g., acetaminophen, codeine, propoxyphene, fentanyl, hydromorphone, morphine, oxycodone, pentazocine, tramadol, and hydrocodone) and biologic response modifiers (e.g., etanercept, adalimumab, anakinra, abatacept, efalizumab, infliximab, l rituximab, and natalizumab)

In certain embodiments, the present invention provides a kit comprising: a) one or more single dosage forms of a compound of the invention; b) one or more single dosage forms of an agent suitable for the treatment or prevention of a rheumatological condition; and c) instructions for the administration of the compound of the invention and the agent suitable for the treatment or prevention of a rheumatological condition.

The present invention provides a kit comprising:
a) a pharmaceutical formulation (e.g., one or more single dosage forms) comprising a compound of the invention; and
b) instructions for the administration of the pharmaceutical formulation, e.g., for treating or preventing a condition as discussed above, e.g., a rheumatological condition.

In certain embodiments, the kit further comprises instructions for the administration of the pharmaceutical formulation comprising a compound of the invention conjointly with an agent suitable for the treatment or prevention of a rheumatological condition. In certain embodiments, the kit further comprises a second pharmaceutical formulation (e.g., as one or more single dosage forms) comprising an agent suitable for the treatment or prevention of a rheumatological condition.

The present invention provides a kit comprising:
a) a first pharmaceutical formulation (e.g., one or more single dosage forms) comprising an agent suitable for the treatment or prevention of a rheumatological condition; and
b) instructions for the administration of the first pharmaceutical formulation with a compound of the invention, e.g., for treating or preventing a condition as discussed above, e.g., a rheumatological condition.

### Dermatological Conditions

The present invention provides a method of treating or preventing a dermatological condition in a patient comprising administering a compound of the invention. In certain embodiments, the dermatological condition may be selected from scleroderma, psoriasis, urticaria, vasculitis, seborrheic dermatitis, pustular dermatosis, eczema, dermatitis, ulcers and erosions resulting from trauma, burns, bullous disorders, or ischemia of the skin or mucous membranes, ichthyoses, epidermolysis bullosae, hypertrophic scars and keloids, cutaneous changes of intrinsic aging and photoaging, frictional blistering caused by mechanical shearing of the skin, cutaneous atrophy resulting from the topical use of corticosteroids, inflammation to mucous membranes (e.g., cheilitis, chapped lips, nasal irritation, or vulvovaginitis), dandruff, sunburn, poison ivy, atopic dermatitis, acne, or rosacea.

In certain embodiments, different compounds of the invention may be conjointly administered with one or more other compounds of the invention. Moreover, such combinations may be conjointly administered with other therapeutic agents, such as other agents suitable for the treatment or prevention of a dermatological condition, such as the conditions disclosed herein. Exemplary agents suitable for the treatment or prevention of dermatological conditions include immunosuppressive agents, such as cyclosporine or calcineurin inhibitors (e.g., tacrolimus or pimecrolimus), corticosteroids (e.g., prednisone and betamethasone dipropionate), antihistamines (e.g., diphenhydramine, hydroxyzine, and cyproheptadine), salicyclic acid, anthroline, calcipotriene, and tazarotene.

In certain embodiments, the present invention provides a kit comprising: a) one or more single dosage forms of a compound of the invention; b) one or more single dosage forms of an agent suitable for the treatment or prevention of a dermatological condition; and c) instructions for the administration of the compound of the invention and the agent suitable for the treatment or prevention of a dermatological condition.

The present invention provides a kit comprising:
a) a pharmaceutical formulation (e.g., one or more single dosage forms) comprising a compound of the invention; and
b) instructions for the administration of the pharmaceutical formulation, e.g., for treating or preventing a condition as discussed above, e.g., a dermatological condition.

In certain embodiments, the kit further comprises instructions for the administration of the pharmaceutical formulation comprising a compound of the invention conjointly with an agent suitable for the treatment or prevention of a dermatological condition. In certain embodiments, the kit further comprises a second pharmaceutical formulation (e.g., as one or more single dosage forms) comprising an agent suitable for the treatment or prevention of a dermatological condition.

The present invention provides a kit comprising:
a) a first pharmaceutical formulation (e.g., one or more single dosage forms) comprising an agent suitable for the treatment or prevention of a dermatological condition; and
b) instructions for the administration of the first pharmaceutical formulation with a compound of the invention, e.g., for treating or preventing a condition as discussed above, e.g., a dermatological condition.

### Neurological Conditions

The present invention provides a method of treating or preventing a neurological condition in a patient comprising administering a compound of the invention. In certain embodiments, the neurological condition may be selected from neurodegeneration or dementia associated with HIV infection, depression, Alzheimer's disease, Parkinson's disease, addiction, alcohol-related disorders, decision analysis, degenerative neurological disorders, dementia, neurological disorders, neuromuscular disorders, psychiatric disorders, brain injury, trauma, neuronal inflammation, or multiple sclerosis.

In certain embodiments, different compounds of the invention may be conjointly administered with one or more other compounds of the invention. Moreover, such combinations may be conjointly administered with other therapeutic agents, such as other agents suitable for the treatment or prevention of a neurological condition, such as the conditions disclosed herein. Exemplary agents suitable for the treatment of neurological conditions include thyroid hormone replacement, cholinesterase inhibitors (e.g., donepezil and tacrine), antipsychotic drugs (e.g., clozapine, olanzapine, quetiapine, risperidone, ziprasidone, aripiprazole, trifluoperazine, flupenthixol, loxapine, perphenazine, chlorpromazine, haloperidol, fluphenazine decanoate, and thioridazine), anxiolytics, such as benzodiazepines (e.g., lorazepam, clonazepam, alprazolam, and diazepam) and non-benzodiazepines (e.g., buspirone), drugs for the treatment of Alzheimer's disease, such as acetylcholinesterase inhibitors (e.g., donepezil, rivastigmine, and galantamine) or NMDA receptor antagonists (e.g., memantine), antidepressants, such as selective serotonin reuptake inhibitors (e.g., fluoxetine, paroxetine, escitalopram, citalopram, and sertraline), serotonin and norepinephrine reuptake inhibitors (e.g., venlafaxine and duloxetine), noradrenergic and specific serotonergic antidepressants (e.g., mirtazapine), norepinephrine reuptake inhibitors (e.g., reboxetine), norepinephrine and dopamine reuptake inhibitors (e.g., bupropion), tricyclic antidepressants (e.g., amitriptyline and desipramine), and monoamine oxidase inhibitors (e.g., phenelzine, moclobemide, and selegiline), drugs for the treatment of multiple sclerosis, such as interferons (e.g., interferon beta-1a and interferon beta-1b), glatiramer acetate, mitoxantrone, natalizumab, and corticosteroids (e.g., methylprednisolone or any of the corticosteroids referenced above), and drugs for the treatment of Parkinson's disease, such as levodopa, carbidopa, benserazide, tolcapone, entacapone; dopamine agonists, such as bromocriptine, pergolide, pramipexole, ropinirole, cabergoline, apomorphine, and lisuride; MAO-B inhibitors, such as selegiline and rasagiline, or combinations thereof.

In certain embodiments, the present invention provides a kit comprising: a) one or more single dosage forms of a compound of the invention; b) one or more single dosage forms of an agent suitable for the treatment or prevention of a neurological condition; and c) instructions for the administration of the compound of the invention and the agent suitable for the treatment or prevention of a neurological condition.

The present invention provides a kit comprising:
a) a pharmaceutical formulation (e.g., one or more single dosage forms) comprising a compound of the invention; and
b) instructions for the administration of the pharmaceutical formulation, e.g., for treating a condition as discussed above, e.g., a neurological condition.

In certain embodiments, the kit further comprises instructions for the administration of the pharmaceutical formulation comprising a compound of the invention conjointly with an agent suitable for the treatment or prevention of a neurological condition. In certain embodiments, the kit further comprises a second pharmaceutical formulation (e.g., as one or more single dosage forms) comprising an agent suitable for the treatment or prevention of a neurological condition.

The present invention provides a kit comprising:
a) a first pharmaceutical formulation (e.g., one or more single dosage forms) comprising an agent suitable for the treatment or prevention of a neurological condition; and
b) instructions for the administration of the first pharmaceutical formulation with a compound of the invention, e.g., for treating or preventing a condition as discussed above, e.g., a neurological condition.

### Cancer

The present invention provides a method of treating or preventing cancer in a patient comprising administering a compound of the invention. In certain embodiments, the cancer may be selected from breast cancer, colon cancer, leukemia, lymphoma, lung cancer, or prostate cancer.

In certain embodiments, different compounds of the invention may be conjointly administered with one or more other compounds of the invention. Moreover, such combinations may be conjointly administered with other therapeutic agents, such as other agents suitable for the treatment or prevention of cancer, such as the chemotherapeutic agents or the combination therapies as disclosed above.

In certain embodiments, a compound of the invention may be conjointly administered with non-chemical methods of cancer treatment or prevention. In certain embodiments, a compound of the invention may be conjointly administered with radiation therapy. In certain embodiments, a compound of the invention may be conjointly administered with surgery, with thermoablation, with focused ultrasound therapy, or with cryotherapy.

The present invention provides a kit comprising:
a) a pharmaceutical formulation (e.g., one or more single dosage forms) comprising a compound of the invention; and
b) instructions for the administration of the pharmaceutical formulation, e.g., for treating or preventing cancer.

In certain embodiments, the kit further comprises instructions for the administration of the pharmaceutical formulation comprising a compound of the invention conjointly with a chemotherapeutic agent, as described above. In certain embodiments, the kit further comprises a second pharmaceutical formulation (e.g., as one or more single dosage forms) comprising a chemotherapeutic agent, as described above.

The present invention provides a kit comprising:
a) a first pharmaceutical formulation (e.g., one or more single dosage forms) comprising a chemotherapeutic agent; and
b) instructions for the administration of the first pharmaceutical formulation with a compound of the invention, e.g., for treating or preventing cancer.

### Infectious Conditions

The present invention provides a method of treating or preventing an infectious condition in a patient comprising administering a compound of the invention. In certain embodiments, the infectious condition may be selected from a bacterial infection, parasitic diseases, a gram negative bacterial infection, salmonella typhimurium infection, an oral infection, a fungal infection, or a viral infection.

The present invention further provides a method of treating or preventing inflammation associated with infection in a patient comprising administering a compound of the invention. In certain such embodiments, compounds of the invention may stimulate and increase production of anti-microbial peptides expressed by human epithelial cells (e.g., bactericidal permeability increasing protein).

In certain embodiments, different compounds of the invention may be conjointly administered with one or more other compounds of the invention. Moreover, such combinations may be conjointly administered with other therapeutic agents, such as other agents suitable for the treatment or prevention of an infectious condition, such as the conditions disclosed herein. Exemplary agents suitable for the treatment or prevention of infectious conditions include aminoglycosides (e.g., amikacin, gentamicin, kanamycin, neomycin, netilmicin, streptomycin, tobramycin, and paromomycin), ansamycins (e.g., geldanamycin and herbimycin), carbacephem (e.g., loracarbef), carbapenems (e.g., ertapenem, doripenem, imipenem, and meropenem), cephalosporins (e.g., cefadroxil, cefazolin, cefalotin, cephalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefdinir, and cefepime), glycopeptides (e.g., vancomycin), macrolides (e.g., azithromycin, clarithromycin, dirithromycin, erythromycin, roxithromycin, troleandomycin, telithromycin, and spectinomycin), monobactams (e.g., aztreonam), penicillins (e.g., amoxicillin, ampicillin, azlocillin, carbenicillin, cloxacillin, dicloxacillin, flucloxacillin, mezlocillin, nafcillin, penicillin, piperacillin, and ticarcillin), polypeptides (e.g., bacitracin, colistin, and polymyxin B), quinolones (e.g., ciprofloxacin, enoxacin, gatifloxacin, levofloxacin, lomefloxacin, moxifloxacin, norfloxacin, ofloxacin, and trovafloxacin), sulfonamides (e.g., mafenide, prontosil, sulfacetamide, sulfamethizole, sulfanilimide, sulfasalazine, sulfisoxazole, and trimethoprim), tetracyclines (e.g., demeclocycline, doxycycline, minocycline, oxytetracycline, and tetracycline), arsphenamine, chloramphenicol, clindamycin, lincoamycin, ethambutol, fosfomycin, fusidic acid, furazolidone, isoniazid, linezolid, metronidazole, mupirocin, nitrofurantoin, platensimycin, pyrazinamide, quinuprisin, dalfopristin, rifampin, and tinidazole.

In certain embodiments, the present invention provides a kit comprising: a) one or more single dosage forms of a compound of the invention; b) one or more single dosage forms of an agent suitable for the treatment or prevention of an infectious condition; and c) instructions for the administration of the compound of the invention and the agent suitable for the treatment or prevention of an infectious condition.

The present invention provides a kit comprising:
a) a pharmaceutical formulation (e.g., one or more single dosage forms) comprising a compound of the invention; and
b) instructions for the administration of the pharmaceutical formulation, e.g., for treating or preventing a condition as discussed above, e.g., an infectious condition.

In certain embodiments, the kit further comprises instructions for the administration of the pharmaceutical formulation comprising a compound of the invention conjointly with an agent suitable for the treatment or prevention of an infectious condition. In certain embodiments, the kit further comprises a second pharmaceutical formulation (e.g., as one or more single dosage forms) comprising an agent suitable for the treatment or prevention of an infectious condition.

The present invention provides a kit comprising:
a) a first pharmaceutical formulation (e.g., one or more single dosage forms) comprising an agent suitable for the treatment or prevention of an infectious condition; and
b) instructions for the administration of the first pharmaceutical formulation with a compound of the invention, e.g., for treating or preventing a condition as discussed above, e.g., an infectious condition.

### Apoptotic Conditions

The present invention provides a method of inhibiting apoptosis in a patient comprising administering a compound of the invention. The present invention further provides a method of treating or preventing a condition arising from apoptosis in a patient comprising administering a compound of the invention. In certain embodiments, the condition arising from apoptosis may be selected from coronary infarct damage, tissue necrosis in chronic inflammation, smooth muscle proliferation disorders (e.g., restenosis following angioplasty), inflammatory states associated with arterial smooth muscle constriction (e.g., coronary spasm, ischemia-induced myocardial injury, cerebral spasm, or cerebral ischemia and related disorders, such as stroke), conditions associated with thrombosis (e.g., coronary thrombosis, phlebitis, or phlebothrombosis), and neurodegenerative diseases.

In certain embodiments, the present invention provides a kit comprising: a) one or more single dosage forms of a compound of the invention; b) one or more single dosage forms of an agent suitable for the treatment or prevention of a condition arising from apoptosis; and c) instructions for the administration of the compound of the invention and the agent suitable for inhibiting apoptosis or for the treatment or prevention of a condition arising from apoptosis.

The present invention provides a kit comprising:
a) a pharmaceutical formulation (e.g., one or more single dosage forms) comprising a compound of the invention; and
b) instructions for the administration of the pharmaceutical formulation, e.g., for inhibiting apoptosis or treating or preventing a condition arising from apoptosis.

In certain embodiments, the kit further comprises instructions for the administration of the pharmaceutical formulation comprising a compound of the invention conjointly with an agent suitable for inhibiting apoptosis or for the treatment or prevention of a condition arising from apoptosis. In certain embodiments, the kit further comprises a second pharmaceutical formulation (e.g., as one or more single dosage forms) comprising an agent suitable for inhibiting apoptosis or for the treatment or prevention of a condition arising from apoptosis.

The present invention provides a kit comprising:
a) a first pharmaceutical formulation (e.g., one or more single dosage forms) comprising an agent suitable for inhibiting apoptosis or for the treatment or prevention of a condition arising from apoptosis; and
b) instructions for the administration of the first pharmaceutical formulation with a compound of the invention, e.g., for inhibiting apoptosis or for treating or preventing a condition arising from apoptosis.

### Angiogenesis

The present invention provides a method of inhibiting angiogenesis in a patient comprising administering a compound of the invention. In certain embodiments, compounds of the present invention limit angiogenesis necessary for solid tumor metastasis. Since angiogenesis and neovascularization are essential steps in solid tumor growth, inhibition of angiogenesis is very useful to prevent the further growth, retard, or even regress solid tumors. Exemplary neoplasias which may be treated with compounds of the present invention include, but are not limited to, gastrointestinal tumors and gliomas.

In certain embodiments, additional disorders or diseases that may be treated or prevented by inhibition of angiogenesis (e.g., by administering a compound of the present invention) include, but are not limited to, retinopathy associated with diabetes, rheumatoid arthritis, osteoarthritis, macular degeneration, glaucoma, Keloid formation, ulcerative colitis, Krohn's disease, and psoriasis.

In certain embodiments, different compounds of the invention may be conjointly administered with one or more other compounds of the invention. Moreover, such combinations may be conjointly administered with other therapeutic agents, such as other agents suitable for inhibiting angiogenesis or for the treatment or prevention of a condition arising from angiogenesis, such as angiogenesis inhibitors including, but not limited to, siRNA's, aptamers, angiostatin, endostatin, and bevacizumab.

In certain embodiments, the present invention provides a kit comprising: a) one or more single dosage forms of a compound of the invention; b) one or more single dosage forms of an agent suitable for inhibiting angiogenesis or for the treatment or prevention of a condition arising from angiogenesis; and c) instructions for the administration of the compound of the invention and the agent suitable for inhibiting angiogenesis or for the treatment or prevention of a condition arising from angiogenesis.

The present invention provides a kit comprising:
a) a pharmaceutical formulation (e.g., one or more single dosage forms) comprising a compound of the invention; and
b) instructions for the administration of the pharmaceutical formulation, e.g., for inhibiting angiogenesis or treating or preventing a condition arising from angiogenesis.

In certain embodiments, the kit further comprises instructions for the administration of the pharmaceutical formulation comprising a compound of the invention conjointly with an agent suitable for inhibiting angiogenesis or for the treatment or prevention of a condition arising from angiogenesis. In certain embodiments, the kit further comprises a second pharmaceutical formulation (e.g., as one or more single dosage forms) comprising an agent suitable for inhibiting angiogenesis or for the treatment or prevention of a condition arising from angiogenesis.

The present invention provides a kit comprising:
a) a first pharmaceutical formulation (e.g., one or more single dosage forms) comprising an agent suitable for inhibiting angiogenesis or for the treatment or prevention of a condition arising from angiogenesis; and
b) instructions for the administration of the first pharmaceutical formulation with a compound of the invention, e.g., for inhibiting angiogenesis or for treating or preventing a condition arising from angiogenesis.

When administered alone or as part of a therapeutic regimen, in certain embodiments, the invention contemplates administration of compounds of the present invention to treat or prevent a particular primary disease, injury, disorder, or condition. In certain embodiments, the invention contemplates administration of compounds of the present invention to treat or prevent symptoms secondary to the primary disease, injury, disorder, or conditions.

### DEFINITIONS

“Angiogenesis” is defined as any enhancement of an existing vascular bed or the formation of new vasculature which benefits tissue perfusion. This includes the formation of new vessels by sprouting of endothelial cells from existing blood vessels or the remodeling of existing vessels to alter size, maturity, direction or flow properties to improve blood perfusion of tissue.

As used herein, the term “corticosteroid” refers to any of the adrenal corticosteroid hormones isolated from the adrenal cortex or produced synthetically, and derivatives thereof that are used for treatment of inflammatory diseases, as described herein. Corticosteroids include those that are naturally occurring, synthetic, or semi-synthetic in origin, and such compounds are characterized by the presence of a steroid nucleus of four fused rings, e.g., as found in cholesterol, dihydroxycholesterol, stigmasterol, and lanosterol structures.

The term “LASIK”, as used herein, is an acronym for LAser in SItu Keratomileusis. This is a type of refractive surgery in which the cornea is reshaped to change its optical power. Specifically, a disc of cornea is raised as a flap, then an excimer laser is used to reshape the middle layer of corneal tissue, producing surgical flattening. LASIK surgery may be used for correcting myopia, hyperopia, and astigmatism.

As used herein, “immunosuppressive agent” refers to agents that suppress the body's ability to elicit an immunological response to the presence of an antigen/allergen. For example, the ability to fight off disease or reject a transplanted organ. Another term for these agents is anti-rejection agents. Not only are they are used to treat organ rejection after transplantation, but many other diseases of immunological etiology such as Crohn's disease, rheumatoid arthritis, lupus, multiple sclerosis, psoriasis, and other diseases and disorders as described herein.

The term “graft”, as used herein, refers to a body part, organ, tissue, or cells. Grafts may comprise all or part of one or more organs such as liver, kidney, heart or lung; body parts such as bone or skeletal matrix; tissue such as skin, intestines, endocrine glands; or progenitor stem cells of various types.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer. Likewise, preferred cycloalkyls have from 3-10 carbon atoms in their ring structure, and more preferably have 5, 6 or 7 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-tirfluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

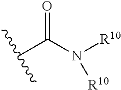

wherein each R¹⁰ independently represent a hydrogen or hydrocarbyl group, or two R¹⁰ are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

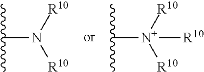

wherein each R¹⁰ independently represents a hydrogen or a hydrocarbyl group, or two R¹⁰ are taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

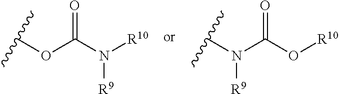

wherein R⁹ and R¹⁰ independently represent hydrogen or a hydrocarbyl group, such as an alkyl group, or R⁹ and R¹⁰ taken together with the intervening atom(s) complete a heterocycle having from 4 to 8 atoms in the ring structure.

The terms “carbocycle”, “carbocyclyl”, and “carbocyclic”, as used herein, refers to a non-aromatic saturated or unsaturated ring in which each atom of the ring is carbon. Preferably a carbocycle ring contains from 3 to 10 atoms, more preferably from 5 to 7 atoms.

The term “carbocyclylalkyl”, as used herein, refers to an alkyl group substituted with a carbocycle group.

The term “carbonate” is art-recognized and refers to a group —OCO₂—R¹⁰, wherein R¹⁰ represents a hydrocarbyl group.

The term “carboxy”, as used herein, refers to a group represented by the formula —CO₂H.

The term “ester”, as used herein, refers to a group —C(O)OR¹⁰ wherein R¹⁰ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “silyl” refers to a silicon moiety with three hydrocarbyl moieties attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

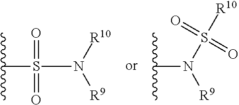

wherein R⁹ and R¹⁰ independently represents hydrogen or hydrocarbyl, such as alkyl, or R⁹ and R¹⁰ taken together with the intervening atom(s) complete a heterocycle having from 4 to 8 atoms in the ring structure.

The term “sulfoxide” is art-recognized and refers to the group —S(O)—R¹⁰, wherein R¹⁰ represents a hydrocarbyl.

The term “sulfonate” is art-recognized and refers to the group SO₃H, or a pharmaceutically acceptable salt thereof.

The term “sulfone” is art-recognized and refers to the group —S(O)₂—R¹⁰, wherein R¹⁰ represents a hydrocarbyl.

The term “thioalkyl”, as used herein, refers to an alkyl group substituted with a thiol group.

The term “thioester”, as used herein, refers to a group —C(O)SR¹⁰ or —SC(O)R¹⁰ wherein R¹⁰ represents a hydrocarbyl.

The term “thioether”, as used herein, is equivalent to an ether, wherein the oxygen is replaced with a sulfur.

The term “urea” is art-recognized and may be represented by the general formula

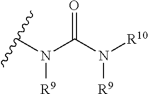

wherein R⁹ and R¹⁰ independently represent hydrogen or a hydrocarbyl, such as alkyl, or either occurrence of R⁹ taken together with R¹⁰ and the intervening atom(s) complete a heterocycle having from 4 to 8 atoms in the ring structure.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxylprotecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPPS groups), glycol ethers, such as ethylene glycol and propylene glycol derivatives and allyl ethers.

The term “healthcare providers” refers to individuals or organizations that provide healthcare services to a person, community, etc. Examples of “healthcare providers” include doctors, hospitals, continuing care retirement communities, skilled nursing facilities, subacute care facilities, clinics, multispecialty clinics, freestanding ambulatory centers, home health agencies, and HMO's.

The term “treating” refers to: preventing a disease, disorder or condition from occurring in a cell, a tissue, a system, animal or human which may be predisposed to the disease, disorder and/or condition but has not yet been diagnosed as having it; stabilizing a disease, disorder or condition, i.e., arresting its development; and relieving one or more symptoms of the disease, disorder or condition, i.e., causing regression of the disease, disorder and/or condition.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

As used herein, a “complex disorder having an inflammatory component” is a disease where the initial pathology/dysfunction in a particular tissue or organ that is vital for the systems biology function of an individual will secondarily lead to systemic metabolic derangement and/or tissue stress causing, or further enhancing, activation of the immune system leading to dysfunction in several organs vital for body homeostasis.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil or injectable organic esters. In a preferred embodiment, when such pharmaceutical compositions are for human administration, the aqueous solution is pyrogen free, or substantially pyrogen free. The excipients can be chosen, for example, to effect delayed release of an agent or to selectively target one or more cells, tissues or organs. The pharmaceutical composition can be in dosage unit form such as tablet, capsule, sprinkle capsule, granule, powder, syrup, suppository, injection or the like. The composition can also be present in a transdermal delivery system, e.g., a skin patch.

A pharmaceutically acceptable carrier can contain physiologically acceptable agents that act, for example, to stabilize or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, boluses, powders, granules, pastes for application to the tongue); absorption through the oral mucosa (e.g., sublingually); anally, rectally or vaginally (for example, as a pessary, cream or foam); parenterally (including intramusclularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatable with such fluids. A preferred route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsuled matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans, and other mammals such as equines, cattle, swine and sheep; and poultry and pets in general.

In certain embodiments, compounds of the invention may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

This invention includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium hydroxide, L-lysine, magnesium hydroxide, 4-(2-hydroxyethyl)morpholine, piperazine, potassium hydroxide, 1-(2-hydroxyethyl)pyrrolidine, sodium hydroxide, triethanolamine, tromethamine, and zinc hydroxide salts. In certain embodiments, contemplated salts of the invention include, but are not limited to, Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In certain embodiments, the invention relates to a method for conducting a pharmaceutical business, by manufacturing a formulation of a compound of the invention, or a kit as described herein, and marketing to healthcare providers the benefits of using the formulation or kit for treating or preventing any of the diseases or conditions as described herein.

In certain embodiments, the invention relates to a method for conducting a pharmaceutical business, by providing a distribution network for selling a formulation of a compound of the invention, or kit as described herein, and providing instruction material to patients or physicians for using the formulation for treating or preventing any of the diseases or conditions as described herein.

In certain embodiments, the invention comprises a method for conducting a pharmaceutical business, by determining an appropriate formulation and dosage of a compound of the invention for treating or preventing any of the diseases or conditions as described herein, conducting therapeutic profiling of identified formulations for efficacy and toxicity in animals, and providing a distribution network for selling an identified preparation as having an acceptable therapeutic profile. In certain embodiments, the method further includes providing a sales group for marketing the preparation to healthcare providers.

In certain embodiments, the invention relates to a method for conducting a pharmaceutical business by determining an appropriate formulation and dosage of a compound of the invention for treating or preventing any of the disease or conditions as described herein, and licensing, to a third party, the rights for further development and sale of the formulation.

### EXAMPLES

Unless otherwise noted, reagents and solvents were used as received from commercial suppliers. Proton nuclear magnetic resonance spectra were obtained on a Bruker AVANCE 300 spectrometer at 300 MHz or on a Bruker AVANCE 500 spectrometer at 500 MHz. Spectra are given in ppm (δ) and coupling constants, J values, are reported in Hertz. Tetramethylsilane was used as an internal standard. Mass spectra were obtained on a Perkin Elmer Sciex 100 atmospheric pressure ionization (APCI) mass spectrometer, or a Finnigan LCQ Duo LC-MS ion trap electrospray ionization (ESI) mass spectrometer. Thin-layer chromatography (TLC) was performed using Analtech silica gel plates, EMD silica gel 60 F₂₅₄ or SAI plastic backed silica gel plates and visualized by ultraviolet (UV) light, iodine, ceric ammonium molybdate or potassium permanganate solution. HPLC analyses were obtained using a BDS C18 column (4.6×250 mm) with UV detection at 254 nm using standard solvent gradient programs (Method 1 and Method 2). Preparative HPLC purifications were performed using a Luna C18 column (21.2×150 mm) with UV detection at 254 nm using various solvent gradient programs and isocratic elutions as described.

### Method 1:

| Time (min) | Flow (mL/min) | % A | % B |
|---|---|---|---|
| 0.0 | 1.0 | 90.0 | 10.0 |
| 20.0 | 1.0 | 0 | 100.0 |
| 35.0 | 1.0 | 0 | 100.0 |

A = Water with 0.05% v/v Trifluoroacetic Acid
B = Acetonitrile with 0.05% v/v Trifluoroacetic Acid.

### Example 1

### Synthesis of Bromoallylic Alcohol Reagent 403

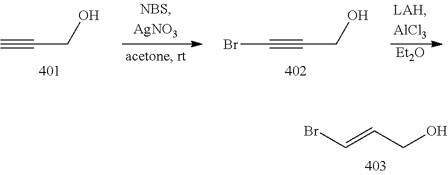

A mixture of propargyl alcohol (401; 3.26 g, 58.2 mmol), N-bromosuccinimide (11.2 g, 62.9 mmol) and silver(I) nitrate (1.00 g, 5.88 mmol) in acetone (100 mL) was stirred at room temperature for 2 h. After this time, the reaction mixture was concentrated and the residue redissolved in iced water (150 mL) and diethyl ether (200 mL), the aqueous layer was removed and extracted with diethyl ether (100 mL). The combined organic layers were washed with brine (100 mL) and dried over sodium sulfate, filtered and concentrated, to give 7.83 g of bromopropargylic alcohol 402 as an orange/yellow oil which was used crude in the next step.

A solution of aluminum trichloride (7.70 g, 57.7 mmol) in diethyl ether (40 mL) was added dropwise to a stirred suspension of lithium aluminum hydride (4.38 g, 115 mmol) in diethyl ether (40 mL) at −5° C., followed by the careful addition of bromopropargylic alcohol (402; 7.38 g, from step 1). The mixture was warmed to room temperature and heated at reflux for 3 h. After this time, the reaction was cooled to room temperature and then to −5° C. Water (4.4 mL) was added carefully and then the reaction mixture was diluted with diethyl ether (100 mL). Sodium hydroxide (15% aqueous, 4.4 mL) was added carefully, followed by water (13 mL). Diethyl ether (100 mL) and magnesium sulfate (10 g) were added, the mixture was stirred for 5 min and then filtered through diatomaceous earth and the filter cake then rinsed with diethyl ether (3×100 mL) and the combined filtrates concentrated. Purification by vacuum distillation (85-100° C., 150 mmHg) afforded the desired bromoallylic alcohol product 403 (3.90 g, 50%) as a colorless oil.

### Example 2

### Synthesis of Phosphonate Building Blocks 411 and 411a

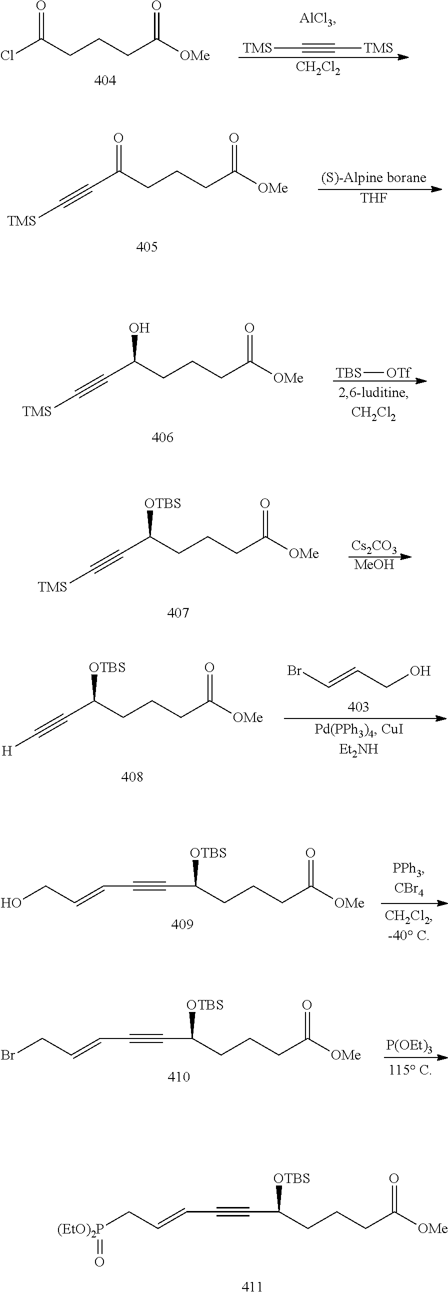

Synthesis of Compound 405. A solution of methyl 4-(chlorocarbonyl)butanoate (404, 23.0 g, 139 mmol) in methylene chloride (40 mL) was added dropwise over 10 min to a suspension of aluminum chloride (22.3 g, 167 mmol) in methylene chloride (130 mL) at 0° C. The mixture was then transferred to a dropping funnel and added to a solution of bis(trimethylsilyl)acetylene (23.7 g, 139 mmol) in methylene chloride (70 mL) at 0° C. The mixture was stirred at 0° C. for 3 h, then poured into a mixture of ice (150 mL) and 0.1 N HCl (150 mL), stirred for 5 min and then diluted with diethyl ether (450 mL) and water (100 mL). The aqueous layer was separated and extracted with diethyl ether (2×150 mL). The combined organic layers were washed with water (300 mL), saturated aqueous sodium bicarbonate (300 mL) and brine (300 mL), dried over sodium sulfate, filtered and concentrated. Purification by flash chromatography (silica, 90:10 hexanes/ethyl acetate) afforded ketone 405 (16.1 g, 51%) as an orange oil.

Synthesis of Compound 406. A mixture of 9-borabicyclo[3.3.1]nonane (26.9 g, 110 mmol) and (1S)-(−)-α-pinene (S-alpine borane; 33.0 g, 242 mmol) was stirred at 65° C. for 3.5 h. The solution was cooled to 0° C. and then a solution of 405 (15.0 g, 66.3 mmol) in tetrahydrofuran was added over 5 min. The reaction was stirred at 0° C. for 20 min and then allowed to warm to room temperature and stirred overnight. The solution was then cooled to 0° C. and acetaldehyde (10.0 mL, 178 mmol) was added and the mixture heated under vacuum at 65° C. for 1 h. The resulting residue was diluted with diethyl ether (120 mL), cooled to 0° C. and stirred with ethanolamine (6.07 g, 99.4 mmol) for 5 min. The cooling bath was removed and the mixture was stirred for an additional 30 min. The resulting precipitate was removed by filtration and the filtrate concentrated to afford a deep yellow oil. Purification by flash chromatography (silica, hexanes to 85:15 hexanes/ethyl acetate) afforded compound 406 (11.9 g, 78%) as a light yellow oil.

Synthesis of Compound 407. To a stirred solution of 406 (12.1 g, 52.9 mmol) in dry methylene chloride (250 mL) at 0° C. under nitrogen was added 2,6-lutidine (12.5 g, 116 mmol). The mixture was stirred for 5 min and then tert-butyldimethylsilyl trifluoromethanesulfonate (20.9 g, 79.5 mmol) was added over 5 min. The reaction was then warmed to room temperature and stirred overnight. The reaction was quenched by adding a saturated aqueous ammonium chloride solution (130 mL), the aqueous layer was separated and then extracted with diethyl ether (2×200 mL). The combined organic layers were dried over magnesium sulfate, filtered and concentrated. Purification by flash chromatography (silica, hexanes to 9:1 hexanes/ethyl acetate) afforded 407 (17.0 g, 93%) as a yellow oil.

Synthesis of Compound 408. To a stirred solution of 407 (14.9 g, 43.5 mmol) in dry methanol (225 mL) under nitrogen was added cesium carbonate (28.3 g, 86.9 mmol) and the mixture was stirred for 25 min. After this time, the reaction was diluted with water (200 mL) and extracted with diethyl ether (3×300 mL). The combined organic layers were dried over magnesium sulfate, filtered and concentrated. Purification by flash chromatography (silica, hexanes to 3:1 hexanes/ethyl acetate) afforded 408 (11.1 g, 94%) as a yellow oil.

Synthesis of Compound 409. To a stirred solution of bromoallylic alcohol (403; 3.44 g, 25.1 mmol) in degassed diethylamine (13 mL) under argon, was added tetrakis(triphenylphosphino) palladium(0) (0.29 g, 0.25 mmol), followed by a solution of 408 (6.78 g, 25.1 mmol) in degassed diethylamine (25 mL). Copper(I) iodide (0.24 g, 1.25 mmol) was added and the reaction mixture stirred for 16 h at room temperature. The reaction mixture was then diluted with diethyl ether (350 mL) and washed with water (4×125 mL) and brine (2×100 mL), dried over sodium sulfate, filtered and concentrated. Purification by flash chromatography (silica, hexanes to 3:1 hexanes/ethyl acetate) afforded 409 (7.33 g, 90%) as an orange oil.

Synthesis of Compound 410. Triphenylphosphine (7.65 g, 29.2 mmol) was added to a stirred solution of 409 (7.33 g, 22.4 mmol) in methylene chloride (250 mL) at −40° C. Carbon tetrabromide (8.92 g, 26.9 mmol) was then added and the mixture maintained between −35 to −45° C. for 1 h. After this time, the reaction mixture was diluted with diethyl ether (500 mL) and saturated aqueous sodium bicarbonate (250 mL). The organic layer was removed and washed with water (200 mL) and brine (200 mL), dried over sodium sulfate, filtered and concentrated. Purification by flash chromatography (silica, hexanes to 5:1 hexanes/ethyl acetate) afforded 410 (7.89 g, 90%) as a pale yellow oil.

Synthesis of Compound 411. A mixture of 410 (7.89 g, 20.2 mmol) and triethylphosphite (30 mL) was heated at 115° C. for 2 h. The reaction was then cooled to room temperature and concentrated in vacuo. Purification by flash chromatography (silica, 5:1 to 1:4 hexanes/ethyl acetate) afforded 411 (8.33 g, 92%) as a pale yellow oil.

Synthesis of Compound 411a. The ethyl ester equivalent of the phosphonate building block 411a was similarly prepared substituting ethyl 4-(chlorocarbonyl)butanoate, for methyl 4-(chlorocarbonyl)butanoate as reagent 404.

### Example 3

### Synthesis of (S)-ethyl 5-hydroxyhept-6-ynoate 412

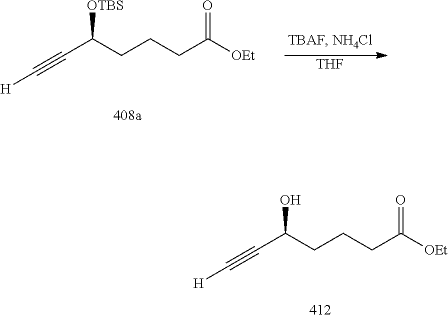

Compound 408a (26.7 g, 104 mmol) and ammonium chloride (5× molar excess) were dissolved in tetrahydrofuran (15 mL) at 0° C. and the tetrabutylammonium floride (5× molar excess of a 1.0 M solution in tetrahydrofuran) was added. The reaction mixture was stirred for 2 h at room temperature. After this time, the reaction was diluted with water (20 mL) and extracted with diethyl ether (2×45 mL). The combined organic layers were washed with brine, dried over sodium sulfate, filtered and concentrated. Purification by silica plug filtration (silica, 95:5 to 80:20 hexanes/ethyl acetate) afforded 412 (15.9 g, 83%) as a yellow oil.

### Example 4

### Synthesis of Aldehyde Building Block 418

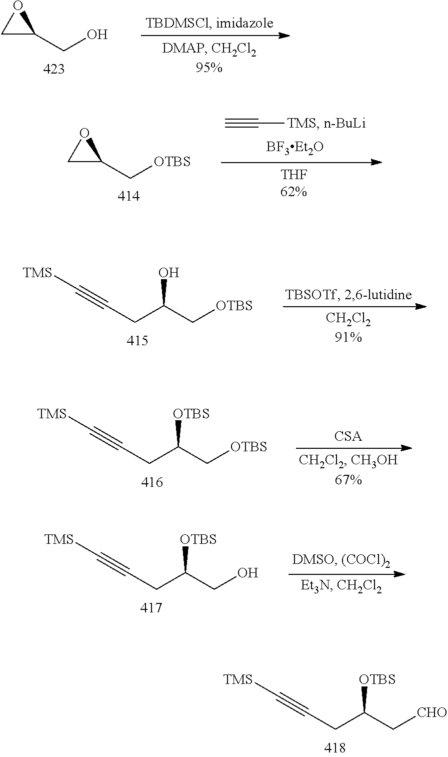

Synthesis of Compound 414. To a solution of commercially available (S)-glycidol (413, 5.10 g, 68.8 mmol) in methylene chloride (40 mL) at 0° C. was added imidazole (6.10 g, 89.5 mmol), followed by 4-dimethylaminopyridine (0.420 g, 3.40 mmol), and then stirred at 0° C. for 15 min. A solution of tert-butylchlorodimethylsilane (10.4 g, 68.8 mmol) in dry methylene chloride (20 mL) was then added dropwise over 5 min. The reaction was stirred at 0° C. for 20 min and then at room temperature for 1 h. After this time, the mixture was quenched with water (100 mL), diluted with diethyl ether (300 mL) and the layers were separated. The aqueous layer was extracted with diethyl ether (2×200 mL) and the combined organic layers were dried over magnesium sulfate, filtered and concentrated. Purification by flash chromatography (silica plug, hexanes to 95:5 hexanes/ethyl acetate) afforded 414 (11.8 g, 92%) as a light yellow oil.

Synthesis of Compound 415. To a stirred solution of trimethylsilylacetylene (4.17 g, 42.5 mmol) in tetrahydrofuran (76 mL) at −78° C. under nitrogen was added a solution of n-butyl lithium in tetrahydrofuran (1.41 M, 15.0 mL, 21.2 mmol) over 10 min. The reaction was stirred at −78° C. for 30 min then a solution of 414 (4.00 g, 21.2 mmol) in tetrahydrofuran (15 mL) was then added, followed by boron trifluoride diethyl etherate (3.10 g, 21.2 mmol). The mixture was then stirred at −78° C. for 30 min and then at room temperature for 1 h. After this time the reaction was quenched by adding a saturated aqueous ammonium chloride solution (40 mL) then diluted with diethyl ether (400 mL). The organic layer was separated, washed with brine (250 mL) and concentrated. Purification by flash chromatography (silica, hexanes to 22:3 hexanes/ethyl acetate) afforded 415 (5.13 g, 84%) as a colorless oil.

Synthesis of Compound 416. To a stirred solution of 415 (6.44 g, 22.4 mmol) in methylene chloride (65 mL) at 0° C. under nitrogen was added 2,6-lutidine (5.29 g, 49.4 mmol) and the mixture stirred for 10 min. Tert-butyldimethylsilyl trifluoromethanesulfonate (8.92 g, 33.7 mmol) was then added slowly over 10 min and the solution was allowed to warm to room temperature overnight. Saturated aqueous ammonium chloride solution (40 mL) was added, then the aqueous layer was separated and then extracted with diethyl ether (200 mL). The combined organic layers were dried over magnesium sulfate, filtered and concentrated. Purification by flash chromatography (silica, hexanes to 9:1 hexanes/ethyl acetate) afforded 416 (8.71 g, 96%) as a light yellow oil.

Synthesis of Compound 417. To a stirred solution of 416 (9.20 g, 22.9 mmol) in methylene chloride (110 mL) and methanol (110 mL) at −5° C. under nitrogen was added (±)-camphor-10-sulfonic acid (5.33 g, 22.9 mmol) and the mixture stirred for 20 min. After this time, the reactions was quenched by adding triethylamine (15 mL) and then concentrated. Purification by flash chromatography (silica, hexanes to 19:1 hexanes/ethyl acetate) afforded 417 (4.41 g, 67%) as a light yellow oil.

Synthesis of Compound 418. To a stirred solution of oxalyl chloride (3.00 g, 23.6 mmol) in methylene chloride (25 mL) at −78° C. under nitrogen was added dropwise a solution of dimethyl sulfoxide (2.20 mL, 30.7 mmol) in methylene chloride (35 mL) followed by stirring at −78° C. for 10 min. A solution of 417 (4.40 g, 15.3 mmol) in methylene chloride (45 mL) was then added and the mixture stirred at −78° C. for 1 h, followed by the addition of triethylamine (7.76 g, 76.7 mmol). The dry ice bath was removed and the reaction was stirred for 45 min. After this time, the reaction was diluted with water (30 mL), the aqueous layer separated and then extracted with diethyl ether (200 mL). The combined organic layers were dried over magnesium sulfate, filtered and concentrated. Purification by flash chromatography (silica, hexanes to 19:1 hexanes/ethyl acetate) afforded 418 (3.91 g, 89%) as a light yellow oil.

### Example 5

### Synthesis of the Vinyl Iodide Building Block 424

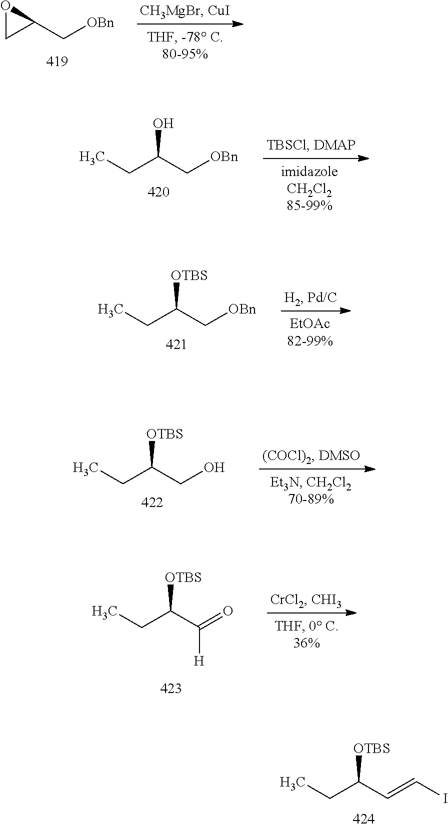

Synthesis of Compound 420. A mixture of finely crushed copper(I) iodide (2.20 g, 11.4 mmol) and tetrahydrofuran (500 mL) was cooled to −78° C. and a solution of methyl magnesium bromide (3.0 M in diethyl ether, 190 mL, 572 mmol) was added dropwise over a period of 30 min. (R)-benzyl glycidol (obtained from benzylation of (S)-glycidol; 419, 18.8 g, 114 mmol) in tetrahydrofuran (60 mL) was then added dropwise over a period of 20 min and the reaction mixture was stirred at −78° C. for 45 min. The reaction was cautiously quenched with saturated aqueous ammonium chloride (300 mL) and then allowed to warm to room temperature. The aqueous layer was removed and extracted with diethyl ether (2×200 mL). The combined organic layers were washed with brine (200 mL), dried over sodium sulfate, filtered and concentrated. Purification by flash chromatography (silica, Isco-330 g, hexanes to 5:1 hexanes/ethyl acetate) afforded 420 (19.3 g, 94%) as a colorless oil.

Synthesis of Compound 421. To a stirred solution of compound 420 (15.2 g, 84.3 mmol) in methylene chloride (60 mL) at 0° C. was added dimethylaminopyridine (0.52 g, 4.22 mmol), imidazole (7.50 g, 109 mmol) and tert-butyldimethylsilyl chloride (12.7 g, 84.3 mmol). The cooling bath was removed and the reaction stirred at room temperature for 3 h. After this time, the mixture was quenched with water (75 mL) and extracted with diethyl ether (2×100 mL). The combined organic layers were washed with brine (100 mL), dried over sodium sulfate, filtered and concentrated. Purification by flash chromatography (silica, Isco-120 g, hexanes to 97:3 hexanes/ethyl acetate) afforded 421 (21.2 g, 86%) as a colorless oil.

Synthesis of Compound 422. Palladium on carbon (1.00 g, 10 wt % (dry basis), 50% water) was added to compound 421 (10.0 g, 33.9 mmol) in ethyl acetate and shaken under an atmosphere of hydrogen (50 psi) at room temperature for 15 h. The reaction mixture was filtered through diatomaceous earth, and the filter cake was washed with ethyl acetate (800 mL). The filtrate was concentrated to afford 422 (6.80 g, 98%) as a light yellow oil.

Synthesis of Compound 423. Oxalyl chloride (4.63 g, 36.4 mmol) was added dropwise to a stirred solution of dimethyl sulfoxide (3.82 g, 48.9 mmol) in methylene chloride (70 mL) under nitrogen at −78° C. The reaction mixture was stirred at −78° C. for 5 min before a solution of compound 422 (5.00 g, 24.5 mmol) in methylene chloride (30 mL) was added over a period of 10 min. The mixture was stirred at −78° C. for 115 min and then triethylamine (11.8 g, 116 mmol) was slowly added. The dry ice bath was removed and the reaction was stirred for 90 min. After this time water (220 mL) was added, the aqueous layer separated and then extracted with diethyl ether (2×300 mL). The combined organic layers were dried over sodium sulfate, filtered and concentrated. Purification by flash chromatography (silica plug, hexanes to 9:1 hexanes/ethyl acetate) afforded 423 (4.30 g, 87%) as a pale yellow oil.

Synthesis of Compound 424. A vigorously stirred suspension of chromium (II) chloride (20.8 g, 170 mmol) in tetrahydrofuran (anhydrous, 150 mL) at 0° C., was treated dropwise with a solution of iodoform (22.5 g, 57.3 mmol) in tetrahydrofuran (40 mL) over a period of 10 min. Compound 423 (4.30 g, 21.3 mmol) in tetrahydrofuran (40 mL) was then added dropwise over a period of 10 min. The mixture was stirred at 0° C. for 110 min, the cooling bath was removed and the reaction was stirred for a further 60 min. The mixture was then poured into water (200 mL) and extracted with ethyl acetate (3×250 mL). The combined organic layers were washed with 10% aqueous sodium thiosulfate (75 mL) and brine (75 mL), dried over sodium sulfate, filtered and concentrated. Trituration with hexanes (4×) before purification removed most of the iodoform. Purification by flash chromatography (Isco-120 g, hexanes) afforded 424, followed by several triturations with hexanes (4×) and then dissolved in ethyl acetate (100 mL) and washed with 10% aqueous sodium thiosulfate (30 mL), dried over sodium sulfate filtered and concentrated to afford 424 (2.87 g, 41%) as a light yellow oil.

### Example 6

### Synthesis of Aldehyde Building Block 425

Synthesis of Compound 425. The O-TBDPS protected aldehyde

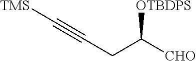

425 was prepared as in Example 4, above, substituting tert-butylchlorodiphenylsilane for tert-butylchlorodimethylsilane in the first reaction step.

### Example 7

### Synthesis of 1-(((R,5E,7E)-8-iodo-1-(trimethylsilyl)octa-5,7-dien-1-yn-4-yloxy)(tert-butyl)(phenyl)silyl)benzene 427

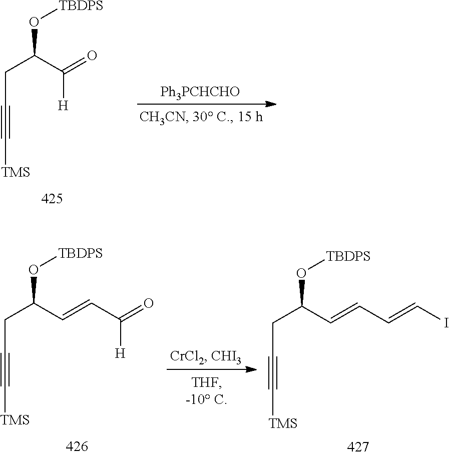

Synthesis of 426. A mixture of 425 (46.6 g, 114 mmol) and (triphenylphosphoranylidene)acetaldehyde (34.8 g, 114 mmol) in acetonitrile (250 mL) was stirred at 30° C. for 15 h. The reaction mixture was concentrated to dryness. The crude product was suspended in hexanes (30 mL) and purified by passing through a plug of silica gel (18×15 cm) and eluted with hexanes (2 L), 2% ethyl acetate/hexanes (20 L), 5% ethyl acetate/hexanes (4 L) to afford 426 (43.2 g, 90%) as a viscous yellow oil.

Synthesis of 427. A dry 1 L flask was charged with chromium (II) chloride (23.4 g, 190 mmol) and heated (with a heat gun) under high vacuum for 15 minutes. The flask was allowed to cool to room temperature under argon, then charged with tetrahydrofuran (250 mL) and sonicated for 15 minutes with occasional stirring. The flask was equipped with a mechanical stirrer, cooled to −10° C. and a solution of iodoform (25.3 g, 64.2 mmol) in tetrahydrofuran (50 mL) was added drop-wise, followed by a solution of 426 (10.1 g, 24.0 mmol) in tetrahydrofuran (50 mL). The reaction mixture was stirred between −10° C. and 0° C. for 1 h under an argon atmosphere and then poured into a mixture of ice and water (600 mL) and diluted with ether (500 mL). The organic layer was separated and the emulsions present in the organic layer were broken by washing with brine (100 mL). The aqueous layer was extracted with ether (2×200 mL) and then the combined organic layers were washed with brine, dried over sodium sulfate, filtered and concentrated. The crude product was purified by passing through a short plug of silica gel (10×4 cm, hexanes) that was pre-treated with 5% triethylamine/hexanes and rinsed with hexanes to afford the desired product which contained iodoform. The resulting yellow residue was triturated with hexanes (4×10 mL) and the combined supernatants were concentrated. This process of trituration was repeated (2×) to afford 427 as a yellow oil (12.32 g). The E:Z ratio of 427 was determined by ¹H NMR as 6.7:1.

Synthesis of Compound 428. The corresponding TBS-protected dienyl iodide

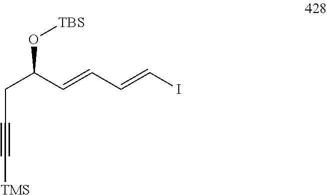

was synthesized as in Example 7, substituting 418 for 425 in the first step.

### Example 8

### Synthesis of Aldehyde Intermediate 432

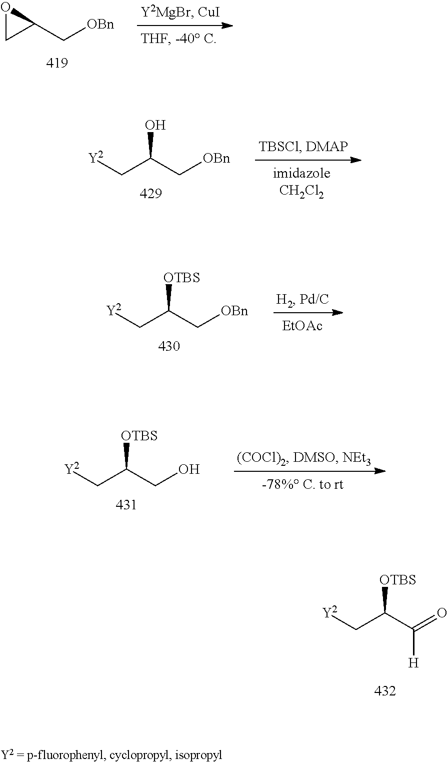

Synthesis of Compound 429. A mixture of finely crushed copper(I) iodide (10% molar amount) and tetrahydrofuran (500 mL) was cooled to −78° C. and a solution of the appropriate magnesium bromide (5× molar excess) was added dropwise over a period of 30 min. Compound 419 in tetrahydrofuran (60 mL) was then added dropwise over a period of 20 min and the reaction mixture was stirred at −78° C. for 45 min. The reaction was cautiously quenched with saturated aqueous ammonium chloride (300 mL) and then allowed to warm to room temperature. The aqueous layer was separated and extracted with diethyl ether (2×200 mL). The combined organic layers were washed with brine (200 mL), dried over sodium sulfate, filtered and concentrated. Purification by flash chromatography (silica, hexanes to 85:15 hexanes/ethyl acetate) afforded 429.

Synthesis of Compound 430. To a stirred solution of compound 429 in methylene chloride (60 mL) at 0° C. was added a 5% molar amount of 4-dimethylaminopyridine, a 1.25× molar excess of imidazole and a molar equivalent of tert-butyldimethylsilyl chloride. The cooling bath was removed and the reaction stirred at room temperature for 3 h. After this time, the mixture was quenched with water (75 mL) and extracted with diethyl ether (2×100 mL). The combined organic layers were washed with brine (100 mL), dried over sodium sulfate, filtered and concentrated. Purification by flash chromatography (silica, hexanes to 96:4 hexanes/ethyl acetate) afforded 430.

Synthesis of Compound 431. Palladium on carbon (10 wt % (dry basis), 50% water) was added to compound 430 in ethyl acetate and shaken under an atmosphere of hydrogen (50 psi) at room temperature until hydrogen uptake had ceased. The reaction mixture was filtered through diatomaceous earth, and the filter cake was washed with ethyl acetate (800 mL). Purification by flash chromatography (silica, hexanes to 80:20 hexanes/ethyl acetate) afforded 431.

Synthesis of Compound 432. Oxalyl chloride (1.5× molar excess) was added dropwise to a stirred solution of dimethyl sulfoxide (2× molar excess) in methylene chloride (70 mL) under nitrogen at −78° C. The reaction mixture was stirred at −78° C. for 5 min before a solution of compound 431 (5.00 g, 24.5 mmol) in methylene chloride (30 mL) was added over a period of 10 min. The mixture was stirred at −78° C. for 115 min and then triethylamine (4.75× molar excess) was slowly added. The dry ice bath was removed and the reaction was stirred for 90 min. After this time water (220 mL) was added, the aqueous layer was separated and then extracted with diethyl ether (2×300 mL). The combined organic layers were dried over sodium sulfate, filtered and concentrated. Purification by flash chromatography afforded 432.

### Example 9

### Synthesis of Compounds 303, 304, 305, 326, 327 and 328

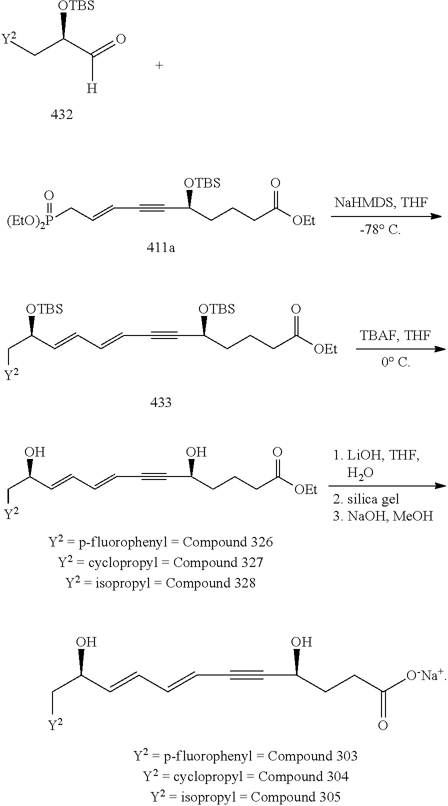

Synthesis of Compound 433. To a stirred solution of phosphonate 411a in tetrahydrofuran (80 mL) at −78° C. was added sodium (bistrimethylsilyl)amide (7.6 mL of a 1.0 M solution in tetrahydrofuran) over 15 min. A solution of the appropriate aldehyde 432 (1.7× molar excess) in tetrahydrofuran (20 mL) was added immediately. The resulting solution was stirred at −78° C. for 2 h and then allowed to warm slowly to 0° C. over 14 h. The reaction was then diluted with diethyl ether (300 mL) and 10% aqueous ammonium chloride solution (100 mL). The aqueous layer was separated and extracted with diethyl ether (2×50 mL). The combined organic layers were washed with brine (100 mL), dried over sodium sulfate, filtered, and concentrated. Purification by flash chromatography (silica, hexanes to 96:4 hexanes/ethyl acetate) afforded 433.

Syntheses of Compounds 326, 327 and 328. Compound 433 was deprotected as described in Example 3 for the synthesis of Compound 412.

Compound 326 was purified by flash chromatography (silica, methylene chloride to 96:4 methylene chloride/methanol) resulting in 78% yield: ¹H NMR (500 MHz, CD₃OD) δ 7.21 (dd, J=8.5, 5.5 Hz, 2H), 7.00-6.93 (m, 2H), 6.53 (dd, J=15.5, 10.8 Hz, 1H), 6.20 (dd, J=15.2, 10.8 Hz, 1H), 5.82 (dd, J=15.2, 6.5 Hz, 1H), 5.62 (d, J=15.5 Hz, 1H), 4.43 (t, J=6.5 Hz, 1H), 4.30 (q, J=6.5 Hz, 1H), 4.12 (q, J=7.1 Hz, 2H), 2.90-2.70 (m, 2H), 2.36 (t, J=7.0 Hz, 2H), 1.80-1.62 (m, 4H), 1.23 (t, J=7.1 Hz, 3H).

Compound 327 was purified by flash chromatography (silica, methylene chloride to 96:4 methylene chloride/methanol) resulting in 59% yield: ¹H NMR (500 MHz, CDCl₃) δ 6.58 (dd, J=15.5, 10.8 Hz, 1H), 6.29 (dd, J=15.2, 10.8 Hz, 1H), 5.86 (dd, J=15.2, 6.5 Hz, 1H), 5.66 (d, J=15.6 Hz, 1H), 4.44 (td, J=6.5, 1.6 Hz, 1H), 4.18 (q, J=6.5 Hz, 1H), 4.12 (q, J=7.1 Hz, 2H), 2.36 (t, J=7.0 Hz, 2H), 1.80-1.62 (m, 4H), 1.53-1.45 (m, 1H), 1.35-1.28 (m, 1H), 1.24 (t, J=7.1 Hz, 3H), 0.80-0.70 (m, 1H), 0.50-0.40 (m, 2H), 0.13-0.00 (m, 2H); HPLC (Method 1) t_{R}=14.8 min., 86.4% (AUC).

Compound 328 was purified by RP preparative chromatography (57:43 to 60:40 methanol/water) resulting in 30% yield: ¹H NMR (500 MHz, CDCl₃) δ 6.56 (dd, J=15.5, 10.8 Hz, 1H), 6.28 (dd, J=15.2, 10.8 Hz, 1H), 5.78 (dd, J=15.2, 6.5 Hz, 1H), 5.66 (d, J=15.6 Hz, 1H), 4.44 (td, J=6.5, 1.6 Hz, 1H), 4.16 (q, J=6.5 Hz, 1H), 4.12 (q, J=7.2 Hz, 2H), 2.36 (t, J=7.0 Hz, 2H), 1.80-1.62 (m, 5H), 1.47-1.41 (m, 1H), 1.32-1.21 (m, 4H), 0.92 (dd, J=6.6, 1.8 Hz, 6H).

Syntheses of Compounds 303, 304 and 305. Lithium hydroxide (2× molar excess) was added to a solution of the appropriate methyl ester in tetrahydrofuran (1.6 mL) and water (0.4 mL). After stiffing at room temperature for 15 h, the reaction mixture was concentrated and run through a small silica plug (silica, dichloromethane to 85:15 dichloromethane/methanol). The resulting free acid was dissolved in methanol (2 mL) and sodium hydroxide (0.1 M in methanol, 1.35 mL) was added. The solution was concentrated to provide compound the desired sodium salt.

Compound 303 was produced in 95% yield: ¹H NMR (500 MHz, CD₃OD) δ 7.20 (dd, J=8.5, 5.5 Hz, 2H), 7.00-6.93 (m, 2H), 6.52 (dd, J=15.5, 10.8 Hz, 1H), 6.19 (dd, J=15.3, 10.8 Hz, 1H), 5.80 (dd, J=15.2, 6.5 Hz, 1H), 5.60 (d, J=15.5 Hz, 1H), 4.45-4.41 (m, 1H), 4.29 (q, J=6.0 Hz, 1H), 2.85-2.70 (m, 2H), 2.19 (t, J=7.5 Hz, 2H), 1.78-1.60 (m, 4H); ESI MS m/z 355 [M+Na]⁺; HPLC (Method 1) t_{R}=13.3 min., 97.9% (AUC).

Compound 304 was produced in 90% yield: ¹H NMR (500 MHz, CD₃OD) δ 6.56 (dd, J=15.5, 10.8 Hz, 1H), 6.28 (dd, J=15.3, 10.8 Hz, 1H), 5.85 (dd, J=15.2, 6.5 Hz, 1H), 5.64 (d, J=15.5 Hz, 1H), 4.46-4.39 (m, 1H), 4.20-4.14 (m, 1H), 2.19 (t, J=7.1 Hz, 2H), 1.80-1.63 (m, 4H), 1.53-1.45 (m, 1H), 1.35-1.28 (m, 1H), 0.80-0.70 (m, 1H), 0.49-0.38 (m, 2H), 0.11-0.00 (m, 2H); ESI MS m/z 279 [M+Na]⁺; HPLC (Method 1) t_{R}=12.1 min., 98.7% (AUC).

Compound 305 was produced in 95% yield: ¹H NMR (500 MHz, CD₃OD) δ 6.55 (dd, J=15.5, 10.8 Hz, 1H), 6.26 (dd, J=15.2, 10.8 Hz, 1H), 5.78 (dd, J=15.2, 6.6 Hz, 1H), 5.64 (d, J=15.6 Hz, 1H), 4.46-4.41 (m, 1H), 4.15 (q, J=6.5 Hz, 1H), 2.19 (t, J=7.1 Hz, 2H), 1.80-1.64 (m, 5H), 1.48-1.40 (m, 1H), 1.32-1.24 (m, 1H), 0.92 (dd, J=6.6, 2.0 Hz, 6H); ESI MS m/z 279 [M+H]⁻; HPLC (Method 1) t_{R}=13.0 min., 98.9% (AUC).

### Example 10

### Synthesis of Aldehyde Intermediate 437

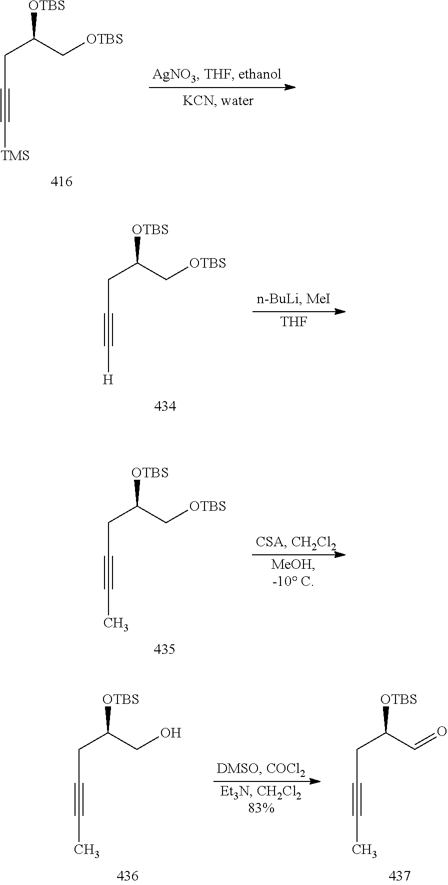

Synthesis of Compound 434. To a stirred solution of compound 416 (1.03 g, 2.56 mmol) in tetrahydrofuran (12.9 mL) and absolute ethanol (6.5 mL) at 0° C. under nitrogen was added dropwise a solution of silver(I) nitrate (0.689 g, 4.06 mmol) in tetrahydrofuran (6.5 mL) and water (6.5 mL) and a yellow precipitate was formed. The ice-bath was replaced with a room temperature water bath and the reaction mixture was stirred for 1.5 h. The reaction mixture was then cooled to 0° C. and a solution of potassium cyanide (0.451 g, 6.92 mmol) in water (6.5 mL) was added dropwise. The ice-bath was removed and the reaction was stirred for 15 min and then filtered through diatomaceous earth. The filter cake was washed with diethyl ether (50 mL), water (50 mL) then with ethyl acetate (50 mL) and finally with water (50 mL). The aqueous layer of the filtrate was separated and extracted with ethyl acetate (50 mL). The combined organic layers were washed with brine (50 mL), dried over magnesium sulfate, filtered and concentrated. Purification by flash chromatography (silica, 95:5 hexanes/ethyl acetate) afforded 434 (0.64 g, 76%) as colorless oil.

Synthesis of Compound 435. To a solution of 434 (0.504 g, 1.53 mmol) in anhydrous tetrahydrofuran (15 mL) at −78° C. was added dropwise n-butyl lithium (1.04 mL 1.77 M in hexanes). After stirring for 25 min, iodomethane (0.19 mL, 3.06 mmol) was added and then the reaction mixture was allowed to warm slowly to room temperature. After stiffing for a further 6 h, the reaction was quenched by the addition of aqueous ammonium chloride. The mixture was extracted with ether (2×50 mL), and the organic layers were combined, dried over sodium sulfate, filtered, and concentrated. Purification by flash chromatography (silica, 95:5 hexanes/ethyl acetate) afforded 435 (0.449 g, 85%) as a light yellow oil.

Synthesis of Compound 436. Compound 435 was deprotected to form Compound 436 as described in Example 4 for the production of Compound 417. Purification by flash chromatography (silica, hexanes to 95:5 hexanes/ethyl acetate) afforded 436 in 55% yield.

Synthesis of Compound 437. Compound 436 was oxidized to Compound 437 as described in Example 4 for the production of Compound 418. Purification by flash chromatography (silica, 94:6 hexanes/ethyl acetate) afforded 437 in 83% yield.

### Example 11

### Synthesis of 1,2-dichloro-4-ethynylbenzene 440

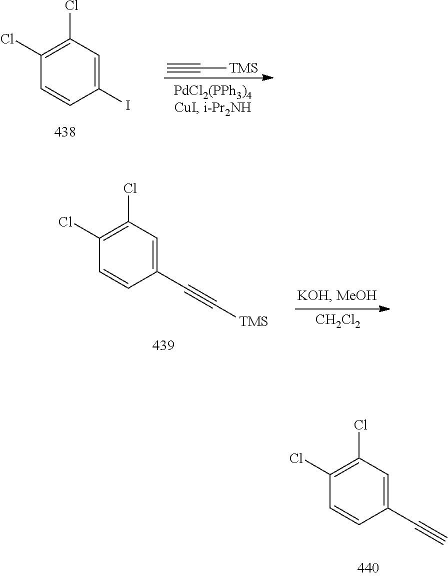

Synthesis of Compound 439. A mixture of 438 (5.03 g, 18.4 mmol), (bistriphenylphosiphino)palladium(II) chloride (0.323 g, 0.461 mmol) and copper(I) iodide (0.088 g, 0.461 mmol) in diisopropylamine (40 mL) was heated to 40° C. and trimethylsilyl acetylene (1.99 g, 20.2 mmol) was added. After stirring at 40° C. for 18 h, the reaction mixture was cooled to room temperature, poured into water (120 mL) and then extracted with methylene chloride (3×40 mL). The combined organic extracts were dried over magnesium sulfate, filtered and concentrated. Purification by flash chromatography (silica, hexanes) afforded 439.

Synthesis of Compound 440. A mixture of 439 (2.54 g, 10.4 mmol) and potassium hydroxide (1.17 g, 20.9 mmol) in methanol (20 mL) and methylene chloride (10 mL) was stirred at room temperature for 1 h. After this time, the reaction mixture was poured into water (30 mL) and extracted with methylene chloride (3×30 mL). The combined organic extracts were dried over magnesium sulfate, filtered and concentrated to afford 440 (1.68 g, 94%) which was used as an appropriate alkyne in Example 12 without further purification.

### Example 12

### Synthesis of Aldehyde Intermediate 444

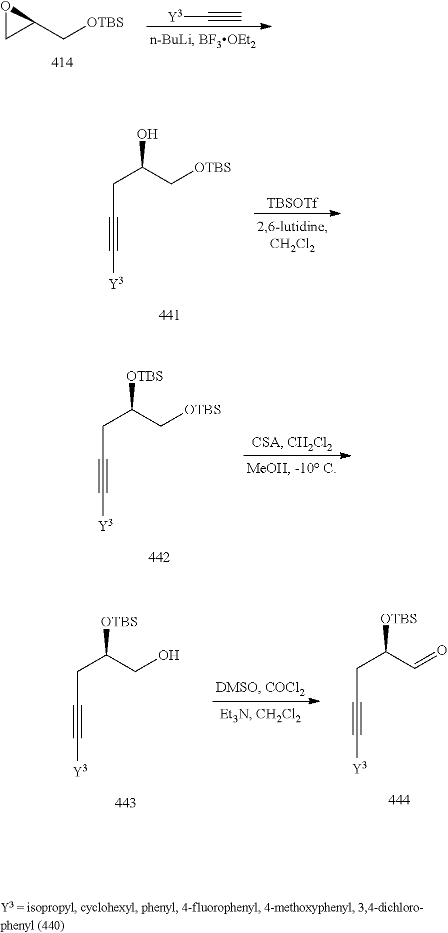

Synthesis of Compound 441. The epoxide opening of compound 414 was performed according to the procedure described for the production of Compound 415 in Example 4 using the appropriate alkyne. Purification by flash chromatography (silica, 95:5 hexanes/ethyl acetate when Y³=isopropy and cyclohexyl; 9:1 hexanes/ethyl acetate when Y³=phenyl, 4-fluorophenyl, 4-methoxyphenyl or 3,4,-dichlorophenyl) afforded 441.

Synthesis of Compound 442. The protection of compound 441 was performed according to the procedure described for the production of Compound 416 in Example 4. Purification by flash chromatography (silica, 95:5 hexanes/ethyl acetate when Y³=isopropy and cyclohexyl; 98:2 hexanes/ethyl acetate when Y³=phenyl; 4:1 hexanes/ethyl acetate when Y³=4-fluorophenyl, 4-methoxyphenyl or 3,4,-dichlorophenyl) afforded 442.

Synthesis of Compound 443. The deprotection of compound 442 was performed according to the procedure described for the production of Compound 417 in Example 4. Purification by flash chromatography (silica, 95:5 hexanes/ethyl acetate when Y³=isopropy and cyclohexyl; 3:7 hexanes/ethyl acetate when Y³=phenyl, 4-fluorophenyl, 4-methoxyphenyl or 3,4,-dichlorophenyl) afforded 443.

Synthesis of Compound 444. The oxidation of compound 443 was performed according to the procedure described for the production of Compound 418 in Example 4. Purification by flash chromatography (silica, 95:5 hexanes/ethyl acetate when Y³=isopropy and cyclohexyl; 9:1 hexanes/ethyl acetate when Y³=phenyl, 4-fluorophenyl, 4-methoxyphenyl or 3,4,-dichlorophenyl) afforded 444.

### Example 13

### Syntheses of Compounds 306, 307, 308, 309, 310, 311, 314, 336, 337, 338, 339, 340, 341 and 342

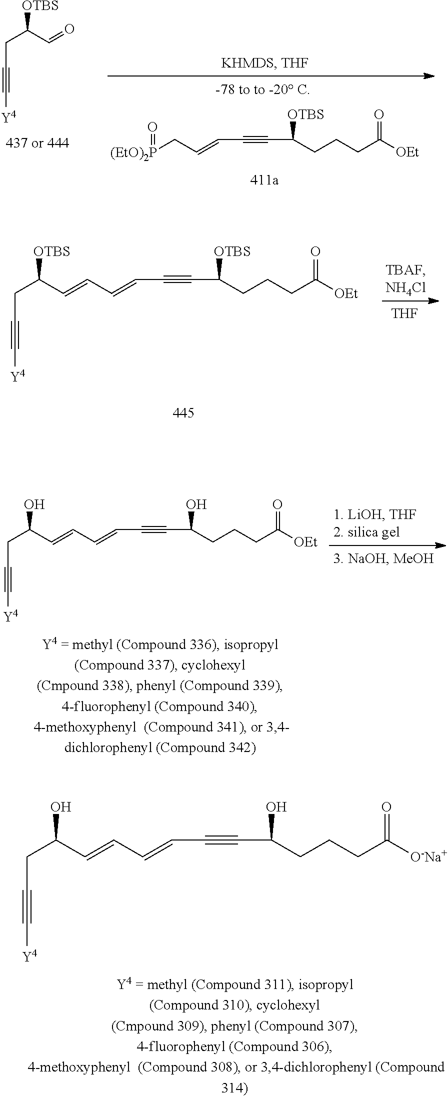

Synthesis of Compound 445. The coupling of compound 437 or 444 with compound 411a was performed according to procedure used to produce compound 433 in Example 9. Purification by flash chromatography (silica, hexanes to 95:5 hexanes/ethyl acetate) afforded 445.

Synthesis of Compounds 336, 337, 338, 339, 340, 341 and 342. The desilylation of compound 445 was performed according to the method used to produce compounds 326, 327 and 328 in Example 9. Purification by flash chromatography (silica, hexanes to 7:3 hexanes/ethyl acetate when Y⁴=isopropyl or cyclohexyl; 3:2 hexanes/ethyl acetate when Y⁴=phenyl, 4-fluorophenyl, 4-methoxyphenyl, or 3,4-dichlorophenyl) afforded the desired compound.

Compound 337: 45% yield: ¹H NMR (500 MHz, MeOD) δ 6.56 (dd, J=15.5, 10.5 Hz, 1H), 6.33 (dd, J=15.2, 10.7 Hz, 1H), 5.87 (dd, J=15.2, 6.2 Hz, 1H), 5.67 (dd, J=15.5, 1.0 Hz, 1H), 4.44 (td, J=6.2, 1.7 Hz, 1H), 4.18 (q, J=6.5 Hz, 1H), 4.12 (q, J=7.0 Hz, 2H), 2.53-2.46 (m, 1H), 2.39 (AB ddd, J=16.5, 5.5, 2.5 Hz, 1H), 2.36 (t, J=7.5 Hz, 2H), 2.29 (AB ddd, J=16.3, 7.2, 2.2 Hz, 1H), 1.81-1.67 (m, 4H), 1.24 (t, J=7.2 Hz, 3H), 1.11 (d, J=6.5 Hz, 6H).

Compound 338: 89% yield: ¹H NMR (500 MHz, MeOD) δ 6.56 (dd, J=15.2, 10.7 Hz, 1H), 6.33 (dd, J=15.0, 11.0 Hz, 1H), 5.87 (dd, J=15.2, 6.2 Hz, 1H), 5.67 (dd, J=15.5, 1.0 Hz, 1H), 4.44 (td, J=6.2, 1.5 Hz, 1H), 4.19 (q, J=6.3 Hz, 1H), 4.12 (q, J=7.2 Hz, 2H), 2.42 (AB ddd, J=16.0, 5.5, 2.0 Hz, 1H), 2.36 (t, J=7.2 Hz, 2H), 2.35-2.28 (m, 2H), 1.79-1.66 (m, 8H), 1.49-1.28 (m, 6H), 1.24 (t, J=7.2 Hz, 3H); ESI MS m/z 395 [M+Na⁺]⁺.

Compound 339: 71% yield: ¹H NMR (500 MHz, CDCl₃) δ 7.45-7.36 (m, 2H), 7.35-7.28 (m, 3H), 6.60 (dd, J=15.6, 10.9 Hz, 1H), 5.92 (dd, J=15.3, 5.9 Hz, 1H), 5.67 (dd, J=15.4, 1.3 Hz, 1H), 4.54 (qd, J=7.2, 1.6 Hz, 1H), 4.44 (pentet, J=5.5 Hz, 1H), 4.13 (q, J=7.2 Hz, 2H), 2.73 (dd, J=16.7, 5.5 Hz, 1H), 2.67 (dd, J=16.7, 6.5 Hz, 1H), 2.37 (t, J=6.7 Hz, 2H), 2.13 (d, J=4.9 Hz, 1H), 1.95 (d, J=5.5 Hz, 1H), 1.88-1.70 (m, 4H), 1.26 (t, J=7.2 Hz, 3H); ¹³C NMR (125 MHz, CDCl₃) δ 173.5, 141.2, 136.6, 131.7, 130.1, 128.3, 128.1, 123.1, 111.3, 92.6, 85.1, 84.1, 83.5, 70.3, 62.6, 60.4, 37.1, 33.8, 28.7, 20.6, 14.2.

Compound 340: 56% yield: ¹H NMR (500 MHz, CDCl₃) δ 7.37 (dd, J=8.8, 5.4 Hz, 2H), 6.99 (t, J=8.7 Hz, 2H), 6.60 (dd, J=15.5, 10.9 Hz, 1H), 6.38 (dd, J=15.2, 10.8 Hz, 1H), 5.91 (dd, J=15.3, 5.9 Hz, 1H), 5.66 (dd, J=15.5, 1.2 Hz, 1H), 4.54 (br q, J=5.5 Hz, 1H), 4.44 (pentet, J=5.3 Hz, 1H), 4.14 (q, J=7.2 Hz, 2H), 2.71 (dd, J=16.7, 5.6 Hz, 1H), 2.66 (dd, J=16.7, 6.5 Hz, 1H), 2.37 (t, J=7.0 Hz, 2H), 2.08 (d, J=4.9 Hz, 1H), 1.94 (d, J=5.4 Hz, 1H), 1.87-1.69 (m, 4H), 1.26 (t, J=7.1 Hz, 3H); ¹³C NMR (125 MHz, CDCl₃) δ 173.5, 161.4, 141.1, 136.5, 133.6, 133.5, 130.2, 119.2, 115.5, 115.4, 111.4, 92.6, 84.8, 84.0, 82.4, 70.3, 62.6, 60.4, 37.1, 33.8, 28.5, 20.6, 14.2.

Compound 341: 78% yield: ¹H NMR (500 MHz, CDCl₃) δ 7.33 (d, J=8.8 Hz, 2H), 6.82 (d, J=8.8 Hz, 2H), 6.60 (dd, J=15.5, 10.9 Hz, 1H), 6.37 (dd, J=15.2, 10.8 Hz, 1H), 5.91 (dd, J=15.3, 5.9 Hz, 1H), 5.65 (dd, J=15.5, 1.2 Hz, 1H), 4.53 (br s, 1H), 4.42 (br s, 1H), 4.14 (q, J=7.1 Hz, 2H), 3.81 (s, 3H), 2.71 (dd, J=16.6, 5.4 Hz, 1H), 2.65 (dd, J=16.6, 6.6 Hz, 1H), 2.37 (t, J=6.7 Hz, 2H), 2.13 (d, J=4.4 Hz, 1H), 1.92 (d, J=4.7 Hz, 1H), 1.87-1.71 (m, 4H), 1.26 (t, J=7.2 Hz, 3H); ¹³C NMR (125 MHz, CDCl₃) δ 173.5, 159.4, 141.2, 136.7, 133.1, 130.0, 122.4, 121.4, 120.0, 119.9, 115.2, 113.9, 111.2, 92.5, 84.1, 83.4, 83.3, 70.3, 62.6, 60.4, 55.3, 37.1, 33.8, 28.7, 20.6, 14.2.

Compound 342: 89% yield: ¹H NMR (500 MHz, CDCl₃) δ 7.48 (d, J=1.9 Hz, 1H), 7.36 (d, J=8.3 Hz, 1H), 7.21 (dd, J=8.3, 1.9 Hz, 1H), 6.59 (dd, J=15.5, 10.9 Hz, 1H), 6.37 (dd, J=15.3, 10.9 Hz, 1H), 5.89 (dd, J=15.2, 6.0 Hz, 1H), 5.67 (dd, J=15.6, 1.4 Hz, 1H), 4.58-4.49 (m, 1H), 4.44 (pentet, J=5.3 Hz, 1H), 4.13 (q, J=7.1 Hz, 2H), 2.72 (dd, J=16.8, 5.6 Hz, 1H), 2.67 (dd, J=16.8, 6.4 Hz, 1H), 2.37 (t, J=6.7 Hz, 2H), 2.01 (d, J=4.8 Hz, 1H), 1.92 (d, J=5.5 Hz, 1H), 1.87-1.70 (m, 4H), 1.26 (t, J=7.2, 3H); ¹³C NMR (125 MHz, CDCl₃) δ 173.5, 141.0, 136.3, 133.4, 132.5, 130.9, 130.3, 123.2, 111.6, 92.8, 84.0, 70.2, 62.6, 60.4, 37.1, 33.8, 28.5, 20.6, 14.3.

Synthesis of Compound 336. The desilylation of compound 445 was performed according to the method used to produce compounds 326, 327 and 328 in Example 9. Purification by flash chromatography (silica, 7:3 to 3:2 hexanes/ethyl acetate) afforded an intermediate that was isomerized by dissolving in methylene chloride (50 mL), adding iodine crystals (0.050 g, 0.197 mmol) and stiffing room temperature for 15 min in a dark hood. Then a 10% (wt/v) solution of aqueous sodium thiosulfate (50 mL) was added. The organic layer was separated and washed with water (2×100 mL), dried over sodium sulfate, filtered, and concentrated. Purification by flash chromatography (silica, 5:45:50 to 20:30:50 methyl tert-butyl ether/hexanes/dichloromethane) and careful peak splitting afforded Compound 336 in 38% yield: ¹H NMR (500 MHz, MeOD) δ 6.56 (dd, J=15.5, 11.0 Hz, 1H), 6.33 (dd, J=15.0, 11.0 Hz, 1H), 5.88 (dd, J=15.2, 5.8 Hz, 1H), 5.68 (d, J=15.5 Hz, 1H), 4.45-4.42 (m, 1H), 4.19 (q, J=6.0 Hz, 1H), 4.12 (q, J=7.0 Hz, 2H), 2.36 (t, J=7.0 Hz, 2H), 2.36-2.26 (m, 2H), 1.79-1.64 (m, 4H), 1.74 (t, J=2.5 Hz, 3H), 1.24 (t, J=7.2 Hz, 3H).

Synthesis of Compounds 306, 307, 308, 309, 310, and 314. The hydrolysis of the foregoing ethyl esters was performed according to the procedure for producing compounds 303, 304 and 305 in Example 9.

Compound 306: 95% yield: ¹H NMR (500 MHz, CD₃OD) δ 7.29 (dd, J=8.8, 5.4 Hz, 2H), 6.93 (t, J=8.8 Hz, 2H), 6.50 (dd, J=15.5, 10.8 Hz, 1H), 6.29 (dd, J=15.2, 10.8 Hz, 1H), 5.83 (dd, J=15.2, 6.2 Hz, 1H), 5.59 (dd, J=15.2, 6.2 Hz, 1H), 4.35 (m, 1H), 4.23 (q, J=6.1 Hz, 1H), 2.54 (dd, J=16.6, 6.1 Hz, 1H), 2.49 (dd, J=16.7, 6.6 Hz, 1H), 2.10 (br t, J=6.9, 2H), 1.72-1.49 (m, 4H); APCI MS m/z 355 [M−H]⁻; HPLC (Method 1), 98.0% (AUC).

Compound 307: 86% yield: ¹H NMR (500 MHz, CD₃OD) δ 7.41-7.32 (m, 2H), 7.32-7.22 (m, 3H), 6.60 (dd, J=15.5, 10.8 Hz, 1H), 6.39 (dd, J=15.3, 10.9 Hz, 1H), 5.94 (dd, J=15.2, 6.2 Hz, 1H), 5.70 (d, J=15.5 Hz, 1H), 4.78-4.40 (m, 1H), 4.33 (q, J=6.1 Hz, 1H), 2.65 (dd, J=16.6, 6.0 Hz, 1H), 2.59 (dd, J=16.6, 6.6 Hz, 1H), 2.19 (t, J=7.1 Hz, 2H), 1.81-1.63 (m, 4H); APCI MS m/z 337 [M−H]⁻; HPLC 97.9% (AUC).

Compound 308: 95% yield: ¹H NMR (500 MHz, CD₃OD) δ 7.19 (d, J=8.8 Hz, 2H), 6.74 (d, J=8.8 Hz, 2H), 6.50 (dd, J=15.5, 10.8 Hz, 1H), 6.29 (dd, J=15.4, 11.0 Hz, 1H), 5.84 (dd, J=15.2, 6.2 Hz, 1H), 5.59 (dd, J=15.6, 1.1 Hz, 1H), 4.34 (m, 1H), 4.22 (q, J=6.1 Hz, 1H), 3.68 (s, 3H), 2.53 (dd, J=16.6, 6.0 Hz, 1H), 2.47 (dd, J=16.6, 6.7 Hz, 1H), 2.10 (t, J=6.9 Hz, 2H), 1.72-1.52 (m, 4H); APCI MS m/z 367 [M−H]⁻; HPLC (Method 1) 95.6% (AUC).

Compound 309: 97% yield: ¹H NMR (500 MHz, MeOD) δ 6.56 (dd, J=15.5, 11.0 Hz, 1H), 6.33 (dd, J=15.2, 10.7 Hz, 1H), 5.87 (dd, J=15.2, 6.2 Hz, 1H), 5.67 (d, J=15.5 Hz, 1H), 4.46-4.44 (m, 1H), 4.19 (q, J=6.3 Hz, 1H), 2.42 (AB ddd, J=16.0, 5.5, 2.0 Hz, 1H), 2.37-2.28 (m, 2H), 2.19 (t, J=7.0 Hz, 2H), 1.77-1.66 (m, 8H), 1.52-1.28 (m, 6H); ESI MS m/z 343 [M−H]⁻; HPLC (Method 1)>97.2% (AUC).

Compound 310: 53% yield: ¹H NMR (500 MHz, MeOD) δ 6.56 (dd, J=15.5, 11.0 Hz, 1H), 6.32 (dd, J=15.2, 10.7 Hz, 1H), 5.86 (dd, J=15.2, 6.2 Hz, 1H), 5.67 (dd, J=14.5 Hz, 1H), 4.45-4.44 (m, 1H), 4.18 (q, J=6.4 Hz, 1H), 2.55-2.46 (m, 1H), 2.39 (AB ddd, J=16.4, 6.0, 2.0 Hz, 1H), 2.30 (AB ddd, J=16.3, 7.2, 2.0 Hz, 1H), 2.20 (t, J=6.5 Hz, 2H), 1.81-1.67 (m, 4H), 1.11 (d, J=7.0 Hz, 6H); ESI MS m/z 303 [M−H]⁻; HPLC (Method 1) 95.7% (AUC).

Compound 311: 89% yield: ¹H NMR (500 MHz, MeOD) δ 6.56 (dd, J=15.5, 10.5 Hz, 1H), 6.32 (dd, J=15.2, 11.2 Hz, 1H), 5.88 (dd, J=15.5, 6.0 Hz, 1H), 5.67 (d, J=15.5 Hz, 1H), 4.45-4.44 (m, 1H), 4.18 (q, J=6.2 Hz, 1H), 2.39-2.27 (m, 2H), 2.19 (t, J=7.2 Hz, 2H), 1.77-1.71 (m, 4H), 1.74 (t, J=2.5 Hz, 3H); ESI MS m/z 299 [M+Na]⁺; HPLC (Method 1) 96.6% (AUC).

Compound 314: 95% yield: ¹H NMR (500 MHz, CD₃OD) δ 7.43 (d, J=1.9 Hz, 1H), 7.35 (d, J=8.3 Hz, 1H), 7.19 (dd, J=8.3, 1.9 Hz, 1H), 6.50 (dd, J=15.5, 10.8 Hz, 1H), 6.29 (dd, J=15.3, 10.9 Hz, 1H), 5.82 (dd, J=15.2, 6.2 Hz, 1H), 5.61 (dd, J=15.6, 1.1 Hz, 1H), 4.38-4.31 (m, 1H), 4.25 (q, J=6.2 Hz, 1H), 2.56 (dd, J=16.8, 6.2 Hz, 1H), 2.52 (dd, J=16.8, 6.3 Hz, 1H), 2.10 (t, J=7.0 Hz, 2H), 1.71-1.53 (m, 4H); APCI MS m/z 405 [M−H]⁻; HPLC (Method 1) 98.5% (AUC).

### Example 14

### Synthesis of Compounds 301 and 343

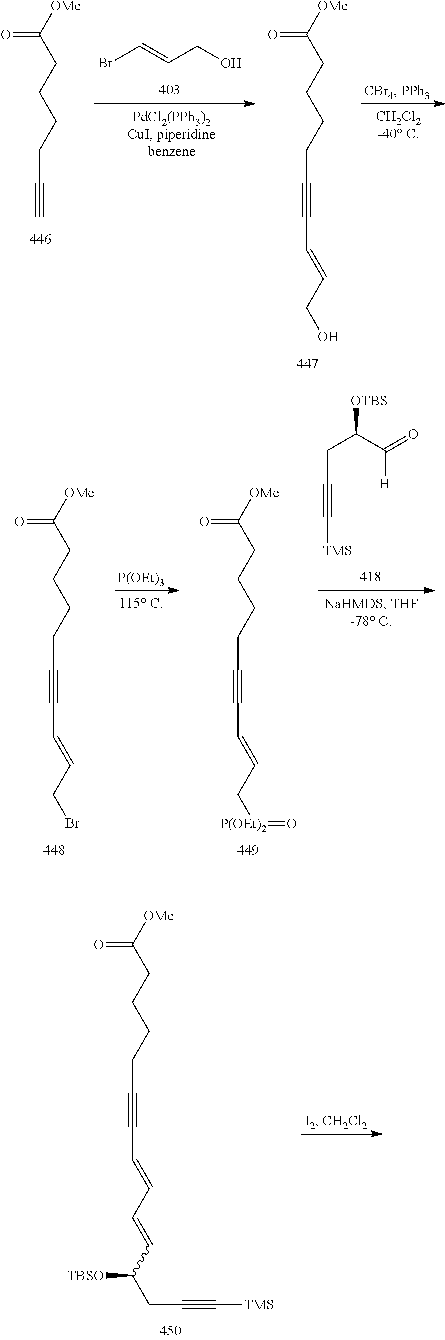

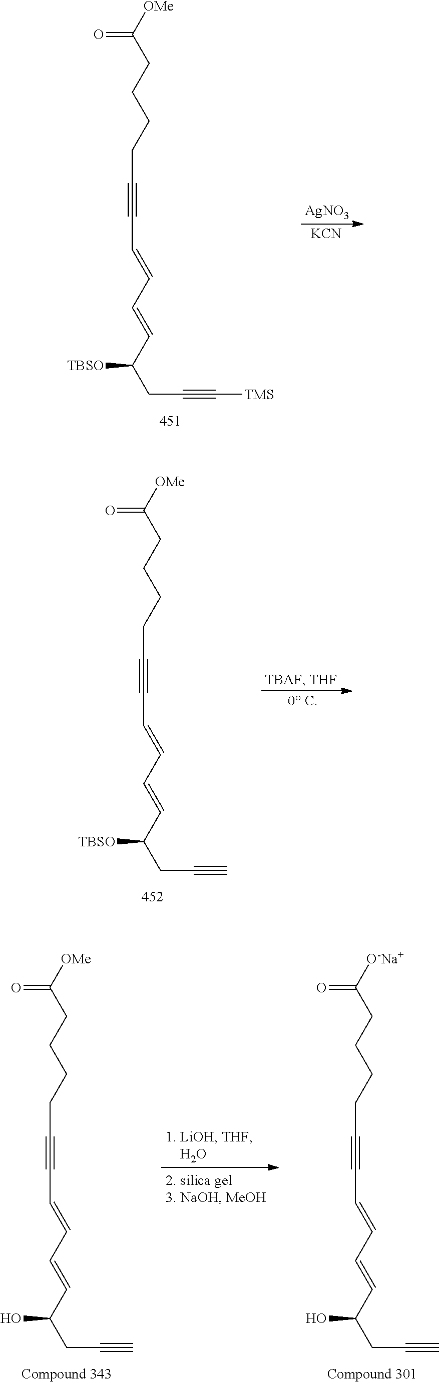

Synthesis of Compound 447. A solution of the acetylene 446 was added to a stirred mixture of the bromo allylic alcohol 403 (1.3× molar excess), bisdiphenylphosphino palladium(II) chloride (0.036 g, 0.05 mmol) and copper(I) iodide (10% molar amount) in benzene (25 mL) under an inert atmosphere of argon. Piperidine (5× molar excess) was then added and the reaction mixture stirred at room temperature and monitored by tlc until complete. The reaction was then diluted with diethyl ether (100 mL) and water (25 mL). The organic layer was separated and washed with brine (50 mL), dried over sodium sulfate, filtered and concentrated. Purification by flash chromatography (silica, hexanes to 3:1 hexanes/ethyl acetate) afforded 447 in 83% yield.

Synthesis of Compound 448. The bromination of compound 447 was performed according to the method used to produce compound 410 in Example 2. Purification by flash chromatography (silica, hexanes to 3:1 hexanes/ethyl acetate) afforded 448 in 89% yield.

Synthesis of Compound 449. The formation of phosphonate 449 from compound 448 was performed according to the method used to produce compound 411 in Example 2. Purification by flash chromatography (silica, hexanes to 3:7 hexanes/ethyl acetate) afforded 449 in 81% yield.

Synthesis of Compound 450. The coupling of compounds 450 and 418 was performed according to the method used to produce compound 433 in Example 9. Purification by flash chromatography (silica, hexanes to 19:1 hexanes/ethyl acetate) afforded 450 in 29% yield.

Synthesis of Compound 451. The isomerisation of compound 450 was performed according to the method used in the production of Compound 336 in Example 13 and afforded 451 in quantitative yield.

Synthesis of Compound 452. To a stirred solution of compound 451 in tetrahydrofuran (12.9 mL) and absolute ethanol (6.5 mL) at 0° C. under nitrogen was added dropwise a solution of silver(I) nitrate (0.689 g, 4.06 mmol) in tetrahydrofuran (6.5 mL) and water (6.5 mL) and a yellow precipitate was formed. The ice-bath was replaced with a room temperature water bath and the reaction mixture was stirred for 1.5 h. The reaction mixture was then cooled to 0° C. and a solution of potassium cyanide (0.451 g, 6.92 mmol) in water (6.5 mL) was added dropwise. The ice-bath was removed and the reaction was stirred for 15 min and then filtered through diatomaceous earth. The filter cake was washed with diethyl ether (50 mL), water (50 mL) then with ethyl acetate (50 mL) and finally with water (50 mL). The aqueous layer of the filtrate was separated and extracted with ethyl acetate (50 mL). The combined organic layers were washed with brine (50 mL), dried over magnesium sulfate, filtered and concentrated. Purification by flash chromatography (silica, hexanes to 85:15 hexanes/ethyl acetate) afforded 452 (0.272 g, 52%) as a colorless oil: ¹H NMR (500 MHz, CDCl₃) δ 6.50 (dd, J=15.5, 10.8 Hz, 1H), 6.24 (dd, J=15.1, 10.8 Hz, 1H), 5.80 (dd, J=15.1, 10.8 Hz, 1H), 5.58 (d, J=15.5 Hz, 1H), 4.31 (q, J=6.3 Hz, 1H), 3.67 (s, 3H), 2.38-2.67 (m, 5H), 1.98 (t, J=2.6 Hz, 1H), 1.80-1.68 (m, 2H), 1.62-1.52 (m, 2H), 0.89 (s, 9H), 0.08 (s, 3H), 0.05 (s, 3H); ¹³C NMR (125 MHz, CDCl₃) δ 173.9, 139.9, 136.6, 129.5, 112.0, 92.5, 81.0, 80.1, 71.5, 70.1, 51.5, 33.6, 28.5, 28.1, 25.8, 24.1, 19.3, 18.2, −4.6, −4.8.

Synthesis of Compound 343. The desilylation of compound 452 was performed according to the method used to produce Compounds 326, 327 and 328 in Example 9. Purification by flash chromatography (silica, hexanes to 85:15 hexanes/ethyl acetate) afforded Compound 343 in 66% yield. ¹H NMR (500 MHz, CDCl₃) δ 6.50 (dd, J=15.5, 10.8 Hz, 1H), 6.32 (dd, J=15.3, 10.8 Hz, 1H), 5.80 (dd, J=15.2, 6.1 Hz, 1H), 5.62 (d, J=15.5 Hz, 1H), 4.35 (dq, J=5.4, 5.4 Hz, 1H), 3.67 (s, 3H), 2.48 (ABdd, J_{AB}=16.6 Hz, J=5.4, 2.6 Hz, 2H), 2.40-2.29 (m, 4H), 2.07 (t, J=2.6 Hz, 1H), 2.01 (d, J=4.8 Hz, 1H), 1.81-1.68 (m, 2H), 1.63-1.49 (m, 2H); ¹³C NMR (125 MHz, CDCl₃) δ 173.9, 139.5, 134.9, 134.7, 112.9, 93.0, 80.0, 79.9, 71.1, 70.1, 51.5, 33.6, 28.1, 27.6, 24.1, 19.3.

Synthesis of Compound 301. The hydrolysis of compound 343 was performed according to the method used to produce compounds 326, 327 and 328 in Example 9. Purification by RP preparative chromatography (3:7 acetonitrile/water) afforded 35 in 17% yield: ¹H NMR (500 MHz, CDCl₃) δ 6.45 (dd, J=15.4 Hz, 10.8 Hz, 1H), 6.30 (dd, J=15.1, 11.2 Hz, 1H), 5.82 (dd, 15.1, 6.2 Hz, 1H), 5.61 (d, J=15.4 Hz, 1H), 4.22 (q, J=6.3 Hz, 1H), 2.38 (obscured ABdd, J_{AB}=16.5 Hz, J=6.0, 2.6 Hz, 2H), 2.33 (td, J=7.1, 1.8 Hz, 2H), 2.27 (td, J=2.7, 0.7 Hz, 1H), 2.17 (t, J=7.5 Hz, 2H), 1.76-1.64 (m, 2H), 1.59-1.48 (m, 2H); ESI MS m/z 245 [M−H]⁻; HPLC (Method 1)>99% (AUC).

### Example 15

### Synthesis of Compounds 302 and 329

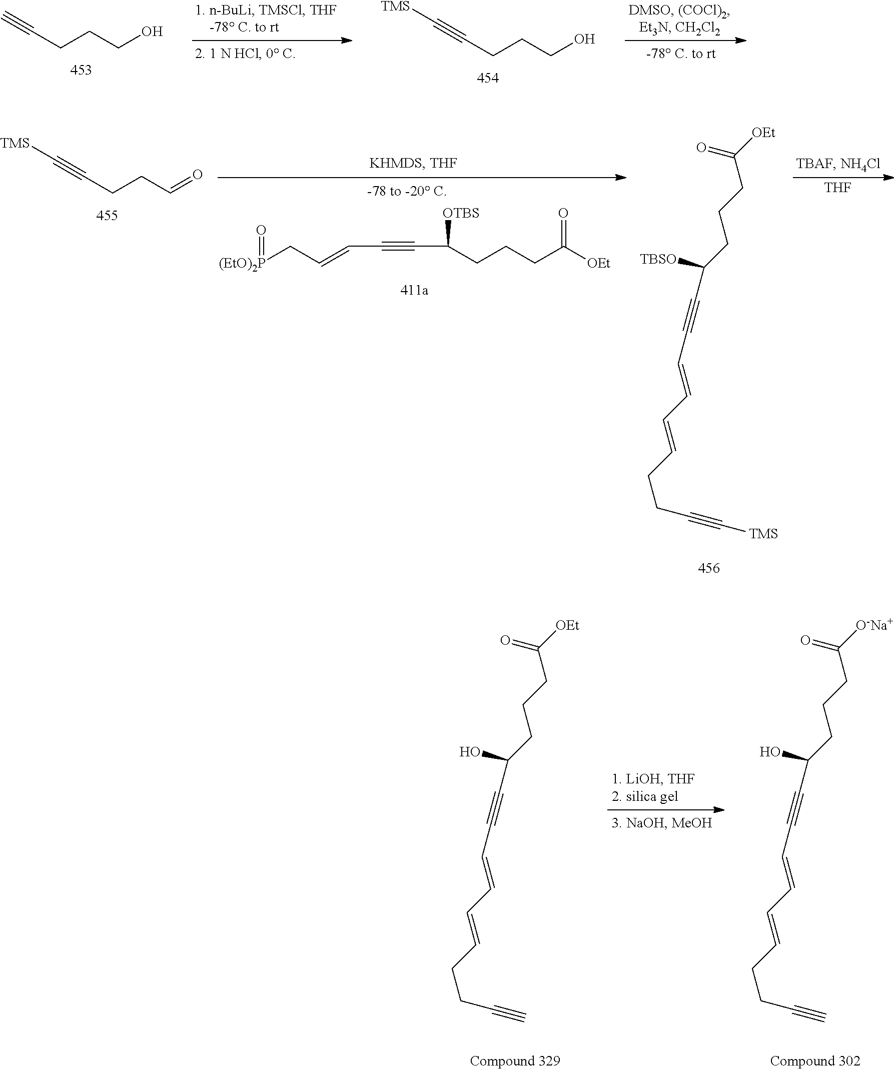

Synthesis of Compound 454. To a solution of 453 (5.64 g, 67.0 mmol) in tetrahydrofuran (60 mL) at −78° C. was added dropwise n-butyl lithium (1.56 M in hexanes, 86.0 mL, 134.0 mmol). The reaction mixture turned into a bright yellow thick gel and after 5 min it became thinner and turned from yellow to white. After a further 25 min, chlorotrimethylsilane (25.4 mL, 201.0 mmol) was added and a white precipitate was generated. The reaction mixture was allowed to warm to room temperature and stirred for 18 h. After this time the reaction mixture was cooled to 0° C., 1 N aqueous hydrochloric acid solution was added and the reaction was monitored by TLC until hydrolysis of the trimethylsilyl ether was complete. The mixture was extracted with diethyl ether (2×100 mL), and the combined organic layers were washed with aqueous sodium bicarbonate solution (75 mL) and brine (75 mL), dried over sodium sulfate, filtered and concentrated. Purification by flash chromatography (silica, 80:20 hexanes/ethyl acetate) afforded 454 (7.90 g, 75%) as a light yellow oil.

Synthesis of Compound 455. The oxidation of compound 454 was performed according to the method used to produce compound 418 in Example 4. Purification by flash chromatography (silica, hexanes to 85:15 hexanes/ethyl acetate) afforded 455 in 75% yield.

Synthesis of Compound 456. Coupling of compound 455 and compound 411a was performed according to the procedure for producing Compound 433 in Example 9 using KHMDS in place of NaHMDS. Purification by flash chromatography (silica, hexanes to 95:5 hexanes/ethyl acetate) and careful peak splitting afforded 456 in 9% yield.

Synthesis of Compound 329. Desilylation of compound 456 was performed according to the method used to produce Compounds 326, 327 and 328 in Example 9. Purification by flash chromatography (silica, 5:1 hexanes/ethyl acetate) afforded Compound 329 in 89% yield: ¹H NMR (500 MHz, CDCl₃) δ 6.56 (dd, J=15.7, 10.7 Hz, 1H), 6.14 (dd, J=15.2, 10.7 Hz, 1H), 5.84 (dt, J=15.5, 6.7 Hz, 1H), 5.54 (d, J=15.5 Hz, 1H), 4.54-4.51 (m, 1H), 4.14 (q, J=7.0 Hz, 2H), 2.39-2.27 (m, 6H), 1.97 (t, J=2.5 Hz, 1H), 1.84-1.73 (m, 4H), 1.26 (t, J=7.5 Hz, 3H).

Synthesis of Compound 302. Hydrolysis of compound 329 was performed according to the method used to produce Compounds 303, 304 and 305 in Example 9 to afford Compound 302 in 95% yield: ¹H NMR (500 MHz, MeOD) δ 6.51 (dd, J=15.5, 10.5 Hz, 1H), 6.17 (dd, J=15.0, 11.0 Hz, 1H), 5.84 (dt, J=15.0, 6.7 Hz, 1H), 5.58 (d, J=15.5 Hz, 1H), 4.44-4.41 (m, 1H), 2.31-2.24 (m, 4H), 2.22 (t, J=2.5 Hz, 1H), 2.19 (t, J=7.0 Hz, 2H), 1.77-1.66 (m, 4H); ESI MS m/z 245 [M−H]⁻; HPLC (Method 1) 98.3% (AUC).

### Example 16

### Synthesis of Compounds 318 and 330

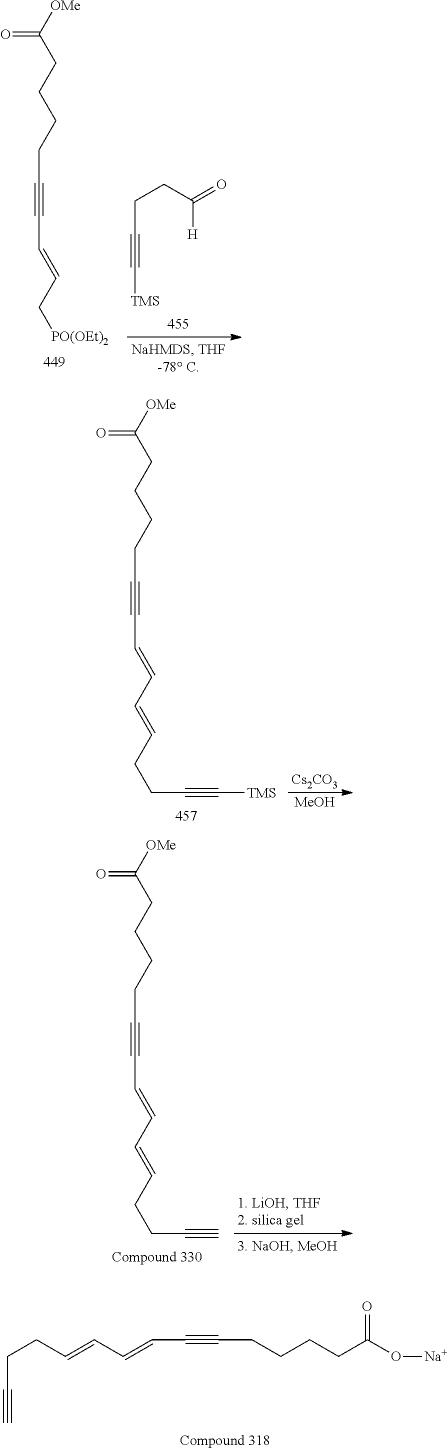

Synthesis of Compound 457. The coupling of compounds 449 and 455 was performed according to the method used to produce compound 433 in Example 9. Purification by flash chromatography (silica, 95:5 hexanes/ethyl acetate) afforded 457 in 57% yield.

Synthesis of Compound 330. The deprotection of compound 457 was performed according to the method used to produce compound 408 in Example 2. Purification by flash chromatography (silica, hexanes to 95:5 hexanes/ethyl acetate) afforded Compound 330 in 23% yield: ¹H NMR (500 MHz, CDCl₃) δ 6.48 (dd, J=15.5, 10.7 Hz, 1H), 6.12 (dd, J=15.1, 10.7 Hz, 1H), 5.80-5.74 (m, 1H), 5.51 (d, J=15.5 Hz, 1H), 3.67 (s, 3H), 2.36-2.28 (m 8H), 1.97 (t, J=2.5 Hz, 1H), 1.78-1.72 (m, 2H), 1.60-1.54 (m, 2H).

Synthesis of Compound 318. The hydrolysis of compound 330 was performed according to the method used to produce compounds 303, 304 and 305 in Example 9 and afforded Compound 318 in 95% yield: ¹H NMR (500 MHz, CD₃OD) δ 6.45 (dd, J=15.5, 10.6 Hz, 1H), 6.17 (dd, J=15.1, 11.1 Hz, 1H), 5.82-5.79 (m, 1H), 5.54 (d, J=15.5 Hz, 1H), 2.36-2.18 (m 9H), 1.76-1.71 (m, 2H), 1.60-1.56 (m, 2H).

ESI MS m/z 229 [M−H]⁻; HPLC (Method 1) 95.5% (AUC).

### Example 17

### Synthesis of Compounds 313 and 331

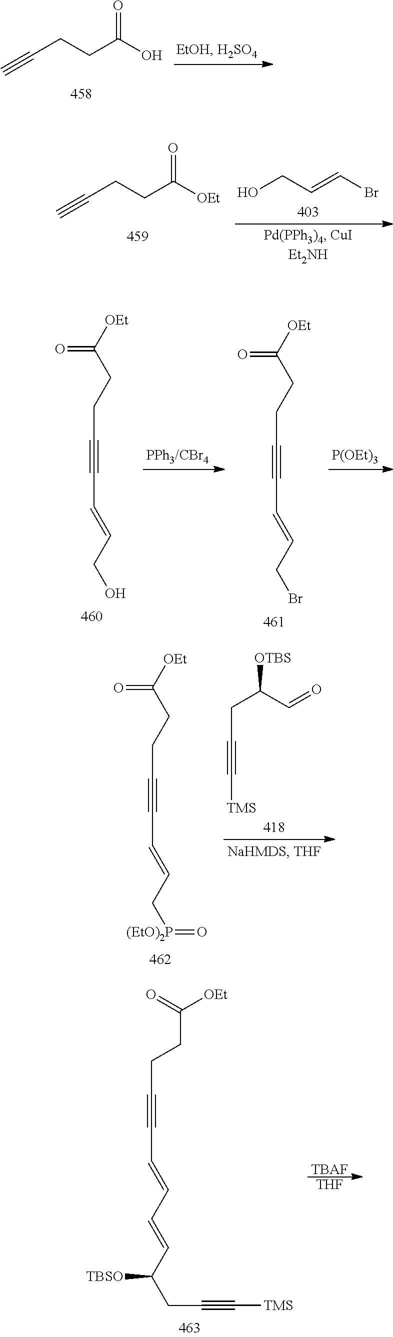

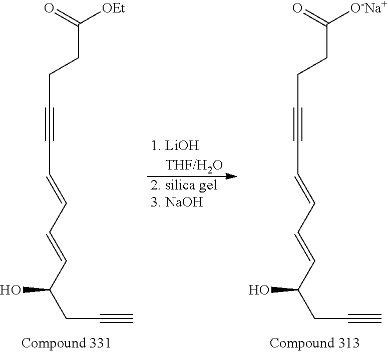

Synthesis of Compound 459. Compound 459 was prepared according to a literature procedure (Tetrahedron, 2000, 56, 5735) and was afforded in 47% yield.

Synthesis of Compound 460. The coupling of compound 459 and 403 was performed according to the method used to produce compound 409 in Example 2. Purification by flash chromatography (silica, hexanes to 2:3 hexanes/ethyl acetate) afforded 460 in 82% yield.

Synthesis of Compound 461. The bromination of compound 460 was performed according to the method used to produce compound 410 in Example 2. Purification by flash chromatography (silica, 4:1 to 2:3 hexanes/ethyl acetate) afforded 461 in 84% yield.

Synthesis of Compound 462. The synthesis of compound 462 was performed according to the method used to produce compound 411 in Example 2. Purification by flash chromatography (silica, hexanes to ethyl acetate) afforded 462 in 90% yield.

Synthesis of Compound 463. The coupling of compound 462 and 418 was performed according to the method used to produce compound 433 in Example 9. Purification by flash chromatography (silica, hexanes to 95:5 hexanes/ethyl acetate) afforded 463 in 49% yield.

Synthesis of Compound 331. The desilylation of compound 463 was performed according to the method used to produce compounds 303, 304, and 305 in Example 9. Purification by flash chromatography (silica, hexanes to 7:3 hexanes/ethyl acetate) afforded Compound 331 in 80% yield: ¹H NMR (500 MHz, C₆D₆) δ 6.62 (dd, J=15.5, 10.9 Hz, 1H), 6.19 (dd, J=15.5, 11.1 Hz, 1H), 5.66 (d, J=15.5 Hz, 1H), 5.60 (dd, J=15.2, 5.7 Hz, 1H), 4.06 (br s, 1H), 4.02 (q, J=6.0 Hz, 2H), 2.61 (td, J=7.3, 2.6 Hz, 2H), 2.37 (t, J=7.3 Hz, 2H), 2.21 (dd, J=2.6, 0.4 Hz, 2H), 1.84 (t, J=2.6 Hz, 1H), 1.70 (br s, 1H), 1.03 (t, J=7.1 Hz, 3H).

Synthesis of Compound 313. The hydrolysis of compound 331 was performed according to the method used to produce compounds 326, 327 and 328 in Example 9 and afforded Compound 313 in 95% yield: ¹H NMR (500 MHz, CD₃OD) δ 6.46 (dd, J=15.4, 10.8 Hz, 1H), 6.30 (dd, J=15.3, 10.8 Hz, 1H), 5.81 (dd, J=15.2, 6.2 Hz, 1H), 5.60 (d, J=15.5 Hz, 1H), 4.22 (q, J=6.1 Hz, 1H), 2.56 (td, J=7.5, 2.0 Hz, 2H), 2.45-2.30 (m, 5H); ESI MS m/z 217 [C₁₃H₁₄O₃−H]⁻; HPLC (Method 1) t_{R}=12.9 min., >99% (AUC).

### Example 18

### Synthesis of Compounds 312 and 332

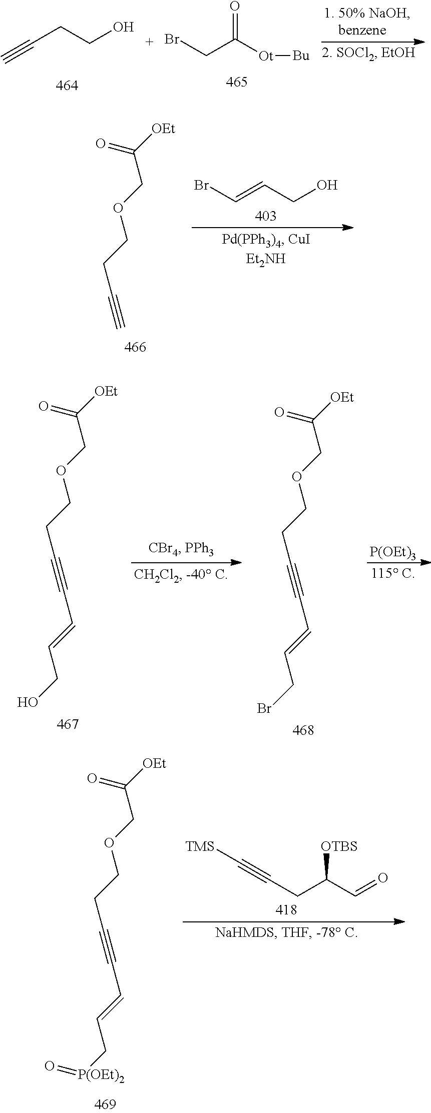

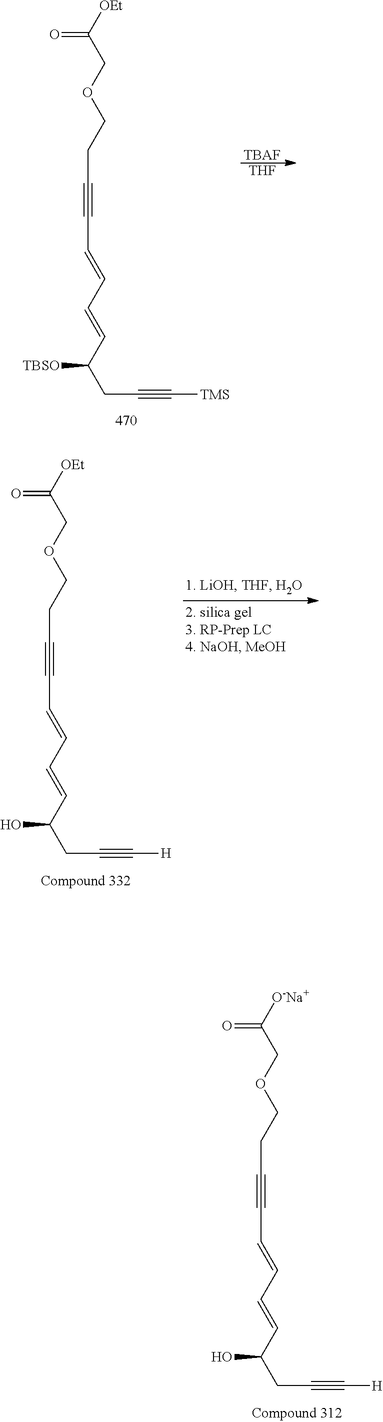

Synthesis of Compound 466. A stirred mixture of 464 (3.00 g, 42.8 mmol) and tetrabutyl ammonium sulfate (1.45 g, 4.28 mmol) in benzene (50 mL) and 50% aqueous sodium hydroxide (50 mL) was cooled to 0° C., followed by the slow addition of 465 (12.5 g, 64.2 mmol). The reaction was then warmed to room temperature and stirred for 18 h. After this time, pentane (100 mL) and water (50 mL) was added, the aqueous layer was separated and extracted with diethyl ether (2×100 mL). The combined organic layers were dried over sodium sulfate, filtered and concentrated. The residue was then dissolved in ethanol (50 mL), cooled to 0° C. and then thionyl chloride (9.3 mL, 128.4 mmol) was added. The reaction mixture was stirred at 0° C. for 1 h, then warmed to room temperature and stirred for a further 18 h and then concentrated. Purification by flash chromatography (silica, Isco-120 g, hexanes to 90:10 hexanes/ethyl acetate) afforded 466 (5.76 g, 86%) as a colorless oil

Synthesis of Compound 467. The coupling of compound 466 with 403 was performed according to the method used to produce compound 409 in Example 2. Purification by flash chromatography (silica, hexanes to 3:2 hexanes/ethyl acetate) afforded 467 in 77% yield.

Synthesis of Compound 468. The bromination of compound 467 was performed according to the method used to produce compound 410 in Example 2. Purification by flash chromatography (silica, hexanes to 9:1 hexanes/ethyl acetate) afforded 468 in 84% yield.

Synthesis of Compound 469. The synthesis of compound 469 was performed according the method used to produce compound 411 in Example 2. Purification by flash chromatography (silica, 4:1 hexanes/ethyl acetate to ethyl acetate) afforded 469 in 89% yield.

Synthesis of Compound 470. The coupling of compounds 469 and 418 was performed according to the method used to produce compound 433 in Example 9. Purification by flash chromatography (silica, hexanes to 9:1 hexanes/ethyl acetate) afforded 470 in 23% yield.

Synthesis of Compound 332. The desilylation of compound 470 was performed according to the method used to produce compounds 326, 327 and 328 in Example 9. Purification by flash chromatography (silica, 9:1 to 1:1 hexanes/ethyl acetate) afforded Compound 332 in 80% yield: ¹H NMR (500 MHz, CD₃OD) δ 6.50 (dd, J=15.5, 10.8 Hz, 1H), 6.32 (dd, J=15.2, 10.8 Hz, 1H), 5.86 (dd, J=15.2, 6.1 Hz, 1H), 5.62 (d, J=15.5 Hz, 1H), 4.27-4.20 (m, 1H), 4.21 (q, J=7.2 Hz, 2H), 4.15 (s, 2H), 3.66 (t, J=6.8 Hz, 2H), 2.62 (td, J=6.8, 2.1 Hz, 2H), 2.41 (ddd, J=16.6, 6.0, 2.6 Hz, 1H), 2.36 (ddd, J=16.6, 6.7, 2.6 Hz, 1H), 2.28 (t, J=2.5 Hz, 1H), 1.28 (t, J=7.2 Hz, 3H).

Synthesis of Compound 312. The hydrolysis of compound 332 was performed according to the method used to produce compounds 303, 304 and 305 in Example 9. Purification by RP preparative chromatography (9:1 to 1:1 water/acetonitrile) afforded Compound 312 in 28% yield: ¹H NMR (500 MHz, CD₃OD) δ 6.49 (dd, J=15.4, 10.8 Hz, 1H), 6.32 (dd, J=15.2, 10.8 Hz, 1H), 5.85 (dd, J=15.2, 6.2 Hz, 1H), 5.62 (d, J=15.5 Hz, 1H), 4.24 (q, J=6.2 Hz, 1H), 3.92 (s, 2H), 3.62 (t, J=7.0 Hz, 2H), 2.63 (td, J=6.8, 1.8 Hz, 2H), 2.41 (dd, J=16.5, 2.6 Hz, 1H), 2.36 (dd, J=16.5, 2.7 Hz, 1H), 2.27 (t, J=2.6 Hz, 1H); ESI MS m/z 271 [M+Na⁺]⁺; HPLC (Method 1)>99% (AUC).

### Example 19

### Synthesis of Phosphonate Intermediate 478

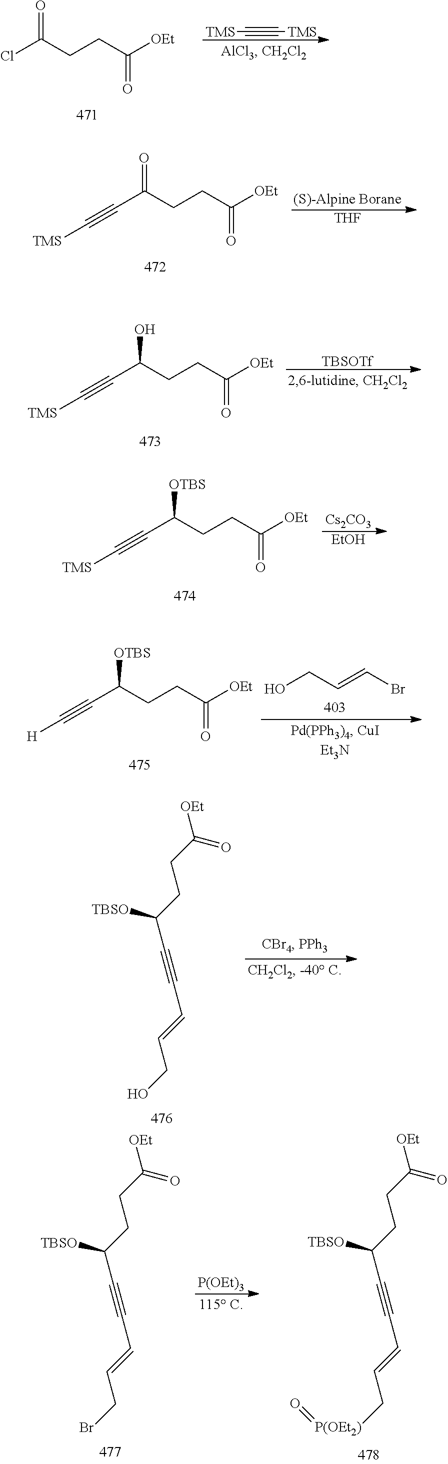

Synthesis of Compound 472. The coupling of ethyl 3-(chlorocarbonyl)propanoate, 471, and bis(trimethylsilyl)acetylene was performed according to the method used to produce compound 405 in Example 2. Purification by flash chromatography (silica, hexanes to 9:1 hexanes/ethyl acetate) afforded 472 in 40% yield.

Synthesis of Compound 473. The chiral reduction of compound 472 was performed according to the method used to produce compound 406 in Example 2. Purification by flash chromatography (silica, hexanes to 95:5 hexanes/ethyl acetate) afforded 473 in 61% yield.

Synthesis of Compound 474. The silylation of compound 473 was performed according to the method used to produce compound 407 in Example 2. Purification by flash chromatography (silica, hexanes to 92:8 hexanes/ethylacetate) afforded 474 in 94% yield. Synthesis of Compound 475. The deprotection of compound 474 was performed according to the method used to produce compound 408 in Example 2. Purification by flash chromatography (silica, hexanes to 9:1 hexanes/ethyl acetate) afforded 475 in 81% yield. Synthesis of Compound 476. The coupling of compound 475 and 403 was performed according to the method used to produce compound 409 in Example 2. Purification by flash chromatography (silica, hexanes to 75:25 hexanes/ethyl acetate) afforded 476 in 93% yield.

Synthesis of Compound 477. The bromination of compound 476 was performed according to the method used to produce compound 410 in Example 2. Purification by flash chromatography (silica, hexanes to 9:1 hexanes/ethyl acetate) afforded 477 in 95% yield. Synthesis of Compound 478. The conversion of compound 477 to the corresponding phosphonate was performed according to the method used to produce compound 411 in Example 2. Purification by flash chromatography (silica, hexanes to 3:7 hexanes/ethyl acetate) afforded 478 in 91% yield.

### Example 20

### Synthesis of Compounds 316 and 333

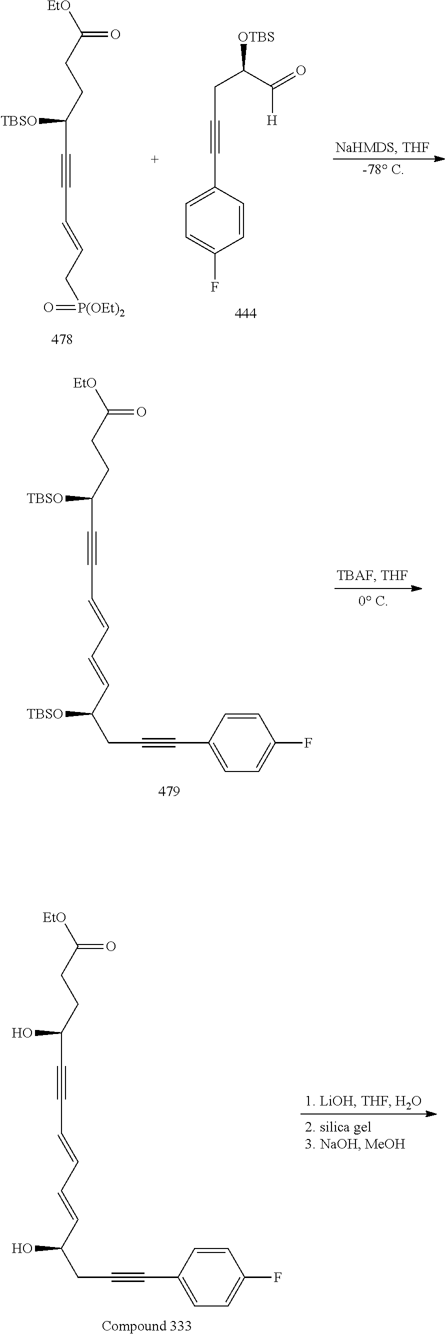

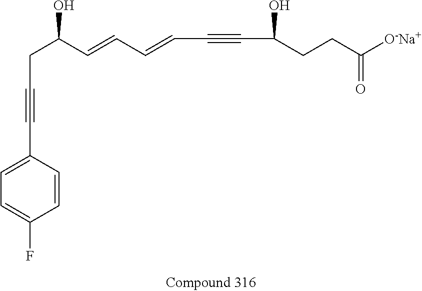

Synthesis of Compound 479. The coupling of compounds 478 (0.22 g, 0.71 mmol) and 444 (0.21 g, 0.47 mmol) was performed according to the method used to produce compound 433 in Example 9. Purification by flash column chromatography (silica, 80:20 hexanes/ethyl acetate) afforded 479 (0.065 g, 25%) as a yellow oil

Synthesis of Compound 333. The deprotection of 479 (0.10 g, 0.17 mmol) was performed according to the method used to produce compounds 326, 327 and 328 in Example 9. Purification by flash column chromatography (silica, 5:40:50 methyl tert-butyl ether/hexanes/methylene chloride to 20:30:50 methyl tert-butyl ether/hexanes/methylene chloride) afforded Compound 333 (0.03 g, 48%) as a colorless oil: ¹H NMR (500 MHz, CDCl₃) δ 7.40 (dd, J=9.0, 5.5 Hz, 2H), 7.03 (t, J=8.5 Hz, 2H), 6.60 (dd, J=15.5, 11.0 Hz, 1H), 6.40 (dd, J=15.0, 11.0 Hz, 1H), 5.96 (dd, J=15.5, 6.5 Hz, 1H), 5.69 (d, J=15.5 Hz, 1H), 4.48-4.51 (m, 1H), 4.34 (q, J=6.0 Hz, 1H), 4.13 (q, J=7.0 Hz, 2H), 2.62 (dd, J=10.5, 6.0 Hz, 2H), 2.47 (t, J=7.5 Hz, 2H), 1.93-1.98 (m, 2H), 1.24 (t, J=7.0 Hz, 3H).

Synthesis of Compound 316. The hydrolysis of compound 333 (0.018 g, 0.05 mmol) was performed according to the method used to produce compounds 303, 304 and 305 in Example 9 and afforded Compound 316 (0.016 g, 89%) as a light yellow film: ¹H NMR (500 MHz, CD₃OD) δ 7.43 (dd, J=8.8, 5.4 Hz, 2H), 7.07 (t, J=8.8 Hz, 2H), 6.65 (dd, J=15.5, 10.9 Hz, 1H), 6.43 (dd, J=15.1, 10.7 Hz, 1H), 5.98 (dd, J=15.2, 6.1 Hz, 1H), 5.73 (d, J=15.5 Hz, 1H), 4.52-4.55 (m, 1H), 4.38 (q, J=6.1 Hz, 1H), 2.65 (dd, J=8.0, 6.1 Hz, 2H), 2.33-2.47 (m, 2H), 1.98 (dd, J=15.0, 7.5 Hz, 2H).

### Example 21

### Synthesis of Compounds 315 and 334

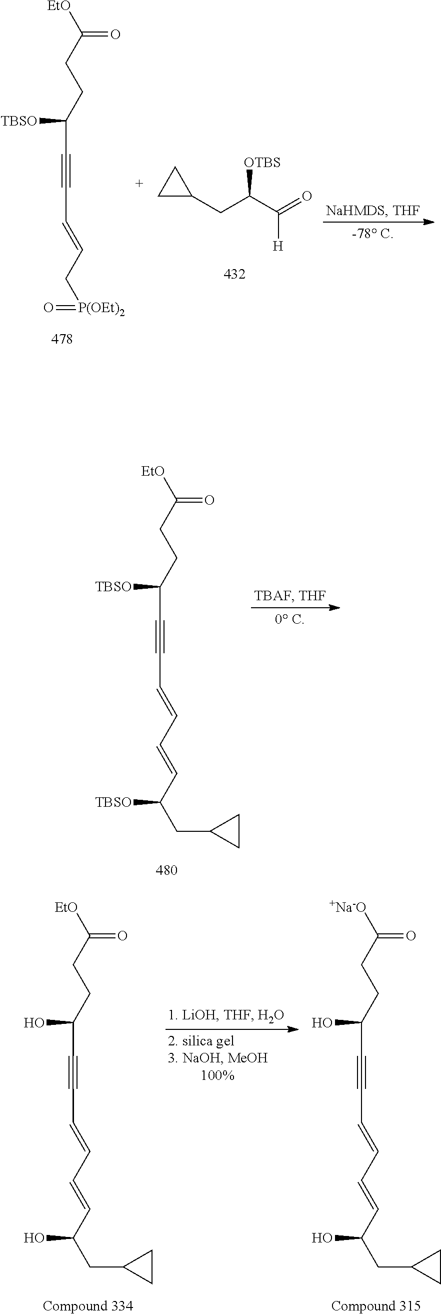

Synthesis of Compound 480. The coupling of 478 and 432 was performed according to the method used to produce compound 433 in Example 9. Purification by flash chromatography (silica, 0 to 50% ethyl acetate/hexanes) afforded 480 in 62% yield.

Synthesis of Compound 334. The deprotection of 480 was performed according to the method used to produce compounds 326, 327 and 328 in Example 9. Purification by flash chromatography (silica, 0 to 2% methanol/methylene chloride) afforded Compound 334 in 31% yield: ¹H NMR (500 MHz, CDCl₃) δ 6.57 (dd, J=15.5, 10.8 Hz, 1H), 6.26 (dd, J=15.0, 10.9 Hz, 1H), 5.87 (dd, J=15.2, 6.3 Hz, 1H), 5.64 (d, J=14.3 Hz, 1H), 4.50 (td, J=6.5, 1.6 Hz, 1H), 4.30 (dd, 12.8, 6.5 Hz, 1H), 4.13 (dd, J=14.2, 7.1 Hz, 2H), 2.48 (t, J=7.4 Hz, 2H), 1.99-1.93 (m, 2H), 1.54-1.47 (m, 2H), 1.31-1.26 (m, 2H), 1.25 (t, J=7.1 Hz, 3H), 0.80-0.72 (m, 1H), 0.50-0.39 (m, 2H), 0.07-0.04 (m, 2H).

Synthesis of Compound 315. The hydrolysis of Compound 334 was performed according to the method used to produce compounds 303, 304 and 305 in Example 9. Purification by flash chromatography (silica, 0 to 15% methanol/methylene chloride) afforded Compound 315 in 100% yield: ¹H NMR (500 MHz, CD₃OD) δ 6.57 (dd, J=15.5, 10.7 Hz, 1H), 6.29 (dd, J=15.2, 10.8 Hz, 1H), 5.85 (dd, J=15.2, 6.4 Hz, 1H), 5.65 (d, J=15.5 Hz, 1H), 4.49 (dt, J=6.3, 1.7 Hz, 1H), 4.18 (dd, J=12.9, 6.5 Hz, 1H), 2.43-2.29 (m, 2H), 1.98-1.92 (m, 2H), 1.54-1.47 (m, 1H), 1.35-1.28 (m, 1H), 0.80-0.72 (m, 1H), 0.48-0.42 (m, 2H), 0.10-0.03 (m, 2H); APCI MS, m/z 263 [M−H]⁻; HPLC (Method 1) 96.7% (AUC).

### Example 22

### Synthesis of Compounds 323 and 335

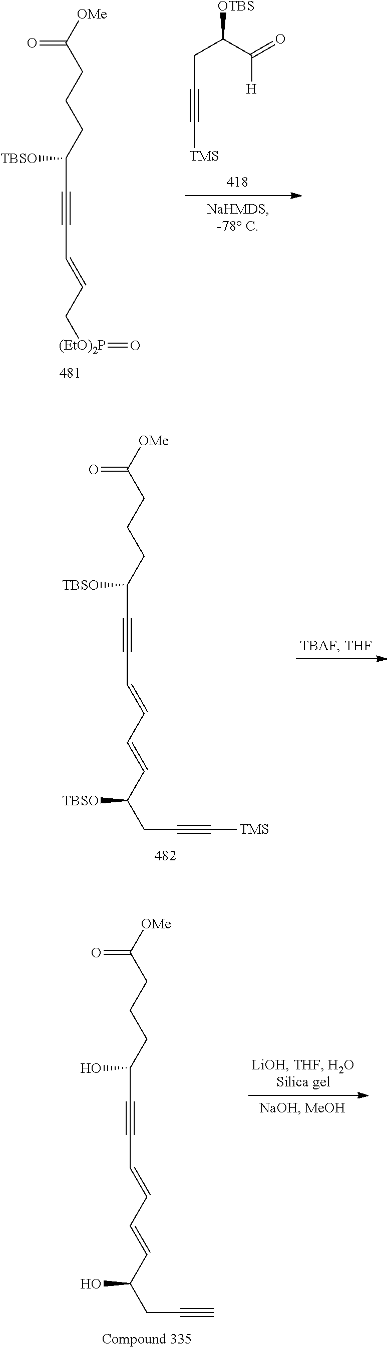

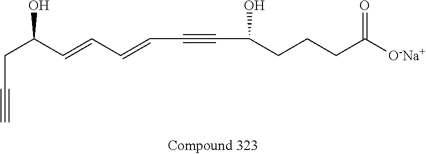

Synthesis of Compound 481. Compound 481 was synthesized in an analogous manner to the scheme for producing Compound 411 according to Example 2, substituting (R)-Alpine Borane for (S)-Alpine Borane in the reduction step.

Synthesis of Compound 482. The coupling of compound 481 and 418 was performed according to the method for producing Compound 433 in Example 9. Purification by flash chromatography (silica, hexanes to 85:15 hexanes/[hexanes/ethylacetate (9:1)] gave lot 1 (0.082 g, 21%) and lot 2 (0.034 g, 9%) of 482, which contained 12% and 3% cis-isomer respectively. Lot 1 was re-subjected to flash chromatography as above to give lot 3 (0.038 g, 10%) which contained <4% cis-isomer.

Synthesis of Compound 335. The desilylation of compound 482 was performed according to the method used to produce compounds 326, 327 and 328 in Example 9. Purification by flash chromatography (silica, hexanes to 60:40 hexanes/ethylacetate) afforded Compound 335 in 61% yield: ¹H NMR (500 MHz, CDCl₃) δ 6.57 (dd, J=15.6, 10.8 Hz, 1H), 6.34 (dd, J=15.3, 10.8 Hz, 1H), 5.86 (dd, J=15.3, 5.9 Hz, 1H), 5.65 (dd, J=15.6, 1.2 Hz, 1H), 4.53 (dq, J=1.6, 6.4 Hz, 1H), 4.37 (dq, J=5.3, 5.3 Hz, 1H), 3.68 (s, 3H), 2.48 (ABdd, J_{AB}=16.6 Hz, J=5.4, 2.6 Hz, 2H), 2.39 (t, J=7.3 Hz, 2H), 2.08 (t, J=2.6 Hz, 1H), 2.04 (d, J=4.9 Hz, 1H), 1.90 (d, J=5.4 Hz, 1H), 1.86-1.68 (m, 4H); ¹³C NMR (125 MHz, CDCl₃) δ 173.9, 141.0, 136.2, 130.2, 111.5, 92.6, 84.0, 79.9, 71.3, 70.0, 62.6, 51.6, 37.0, 33.6, 27.6, 20.6.

Synthesis of Compound 323. The hydrolysis of compound 335 was performed according to the method used to produce compounds 303, 304 and 305 in Example 9 and afforded Compound 323 in 94% yield: ¹H NMR (500 MHz, CDCl₃) δ 6.55 (dd, J=15.5, 10.8 Hz, 1H), 6.34 (dd, J=15.3, 11.1, 1H), 5.88 (dd, J=15.2, 6.1 Hz, 1H), 5.67 (d, J=15.5 Hz, 1H), 4.43 (m, 1H), 4.24 (q, J=6.1 Hz, 1H), 2.39 (ddd, J=16.5, 6.0, 2.6 Hz, 2H), 2.28 (t, J=2.6 Hz, 1H), 2.19 (t, J=7.0 Hz, 2H), 1.80-1.63 (m, 4H). ESI MS m/z 261 [M−H]⁻. HPLC (Method 1)>99% (AUC).

### Example 23

### Synthesis of Phosphonate Intermediate 490

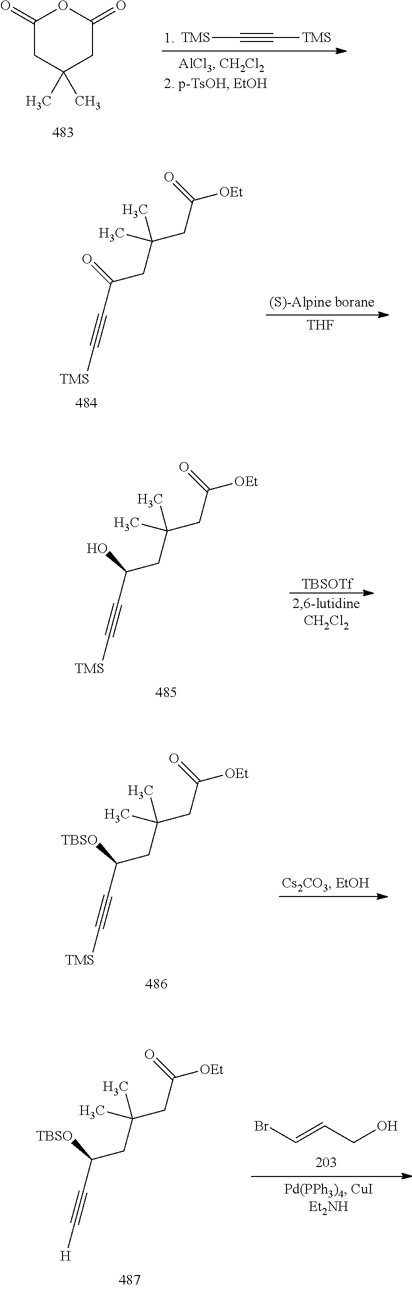

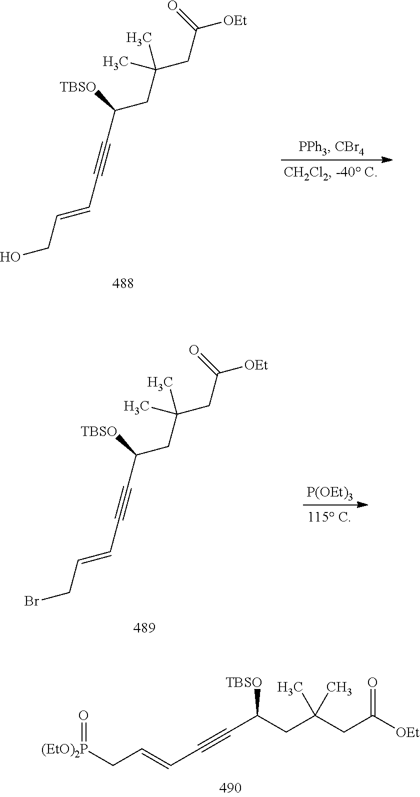

Synthesis of Compound 484. A mixture of 3,3-dimethylglutaric anhydride (483, 1.00 g, 7.03 mmol) and bis trimethylsilyl acetylene (1.20 g, 7.03 mmol) in methylene chloride was cooled to 0° C. and aluminum chloride (0.985 g, 7.38 mmol) was added portion-wise. The reaction mixture was then allowed to warm slowly to room temperature and stirred for 16 h. After this time, the reaction mixture was cooled to 0° C., 1 N hydrochloric acid (25 mL) was added and the mixture stirred for 10 minutes. The organic layer was separated and washed with 1 N hydrochloric acid (2×40 mL), water (40 mL) and brine (40 mL), dried over sodium sulfate, filtered and concentrated. Purification by flash chromatography (silica, 0-15% methanol/methylene chloride) afforded the carboxylic acid (1.46 g, 86%). The carboxylic acid was then dissolved in ethanol (65 mL), para-toluene sulfonic acid (0.115 g, 0.6 mmol) was added and the reaction mixture was stirred at 45° C. for 16 h and then at reflux for a further 24 h. The reaction was quenched by the addition of solid sodium bicarbonate and the volatile solvents were removed. The residue was diluted with diethyl ether (50 mL) and water (100 mL) and the layers were separated. The aqueous layer was extracted with diethyl ether (100 mL) and then the combined layers were washed with brine (100 mL), dried over sodium sulfate, filtered and concentrated. Purification by flash chromatography (silica, 0-10% hexanes/ethyl acetate) afforded 484 (0.76 g, 47%) as a colorless oil.

Synthesis of Compound 485. The reduction of compound 484 was performed according to the method used to produce compound 406 in Example 2. Purification by flash chromatography (silica, 0-8% ethyl acetate/hexanes) afforded 485 in 67% yield.

Synthesis of Compound 486. The protection of compound 485 was performed according to the method used to produce compound 407 in Example 2. Purification by flash chromatography (silica, 0-10% ethyl acetate/hexanes) afforded 486 in 67% yield.

Synthesis of Compound 487. The deprotection of compound 486 was performed according to the method used to produce compound 408 in Example 2. Purification by silica plug (silica, 0-5% ethyl acetate/hexanes) afforded 487 in 95% yield.

Synthesis of Compound 488. The coupling of compound 487 with bromoallylic alcohol 403 was performed according to the method used to produce compound 409 in Example 2. Purification by flash chromatography (silica, 0-25% ethyl acetate/hexanes) afforded 488 in 94% yield.

Synthesis of Compound 489. The bromination of compound 488 was performed according to the method used to produce compound 410 in Example 2. Purification by flash chromatography (silica, 0-10% ethyl acetate/hexanes) afforded 489 in 95% yield.

Synthesis of Compound 490. The synthesis of compound 490 was performed according to the method used to produce compound 411 in Example 2. Purification by flash chromatography (silica, 0-60% ethyl acetate/hexanes) afforded 490 in 95% yield.

### Example 24

### Synthesis of Compounds 344 and 317

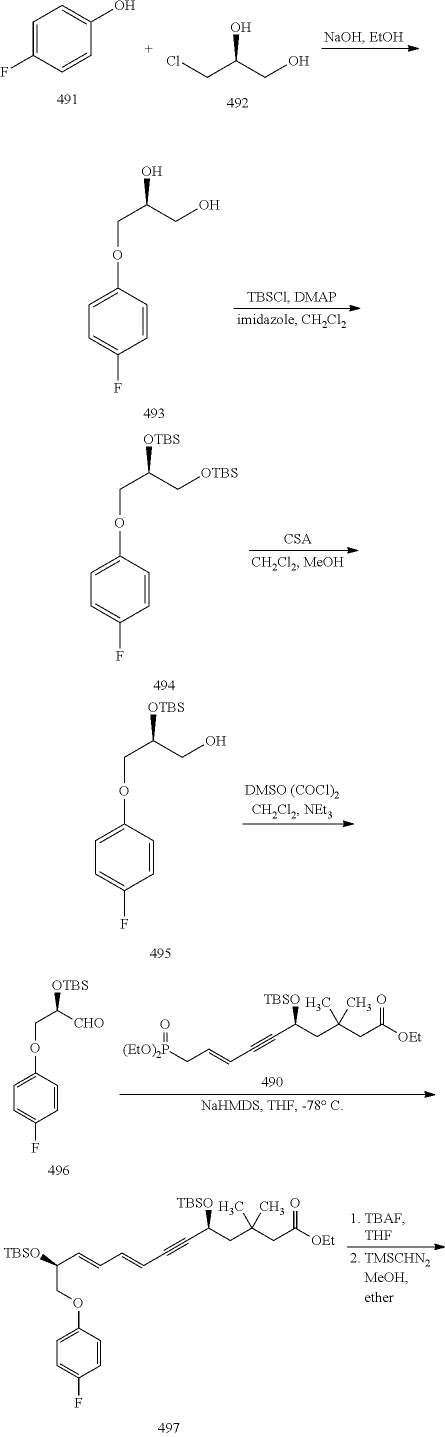

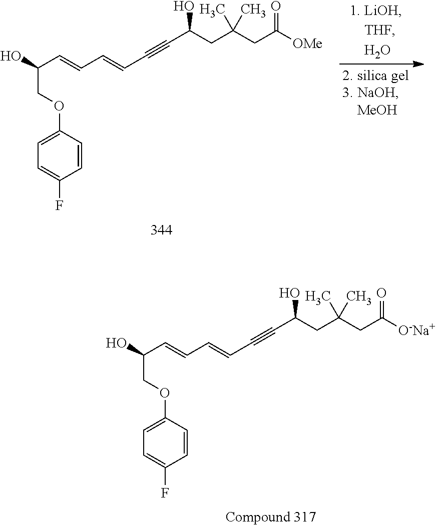

Synthesis of Compound 493. A mixture of 4-fluorophenol (491; 2.36 g, 21.1 mmol) and sodium hydroxide (1.05 g, 26.3 mmol) in ethanol was heated at reflux for 15 minutes and then (S)-3-chloro-1,2-propanediol (492; 2.80 g, 25.3 mmol) was added and the mixture stirred at reflux for 16 h. After this time, the reaction was cooled to room temperature and concentrated. The residue was dissolved in diethyl ether (200 mL) and washed with water (75 mL) and brine (75 mL), dried over sodium sulfate, filtered and concentrated. The residue was crystallized from hexanes/diethyl ether and afforded 493 (3.30 g, 84%) as a white solid.

Synthesis of Compound 494. A mixture of compound 493 (3.05 g, 16.3 mmol) and imidazole (3.34 g, 49.1 mmol) in methylene chloride (40 mL) was cooled to 0° C. and then tert-butyldimethylsilyl chloride (6.17 g, 41.0 mmol) and 4-dimethylaminopyridine (0.20 g, 1.63 mmol) were added. The reaction was stirred at room temperature for 3 h and then diluted with diethyl ether (200 mL) and water (75 mL). The organic layer was separated and then washed with water (75 mL) and brine (75 mL), dried over sodium sulfate, filtered and concentrated. Purification by flash chromatography (silica, 100:0 to 75:25 hexanes/ethyl acetate) afforded 494.

Synthesis of Compound 495. The deprotection of compound 494 (4.38 g, 10.6 mmol) was performed according to the method used to produce compound 417 in Example 4. Purification by flash chromatography (silica, 100:0 to 20:80 hexanes/ethyl acetate) afforded 495 (1.25 g, 39%) as a pale yellow oil.

Synthesis of Compound 496. The oxidation of compound 495 (0.95 g, 3.17 mmol) was performed according to the method used to produce compound 418 in Example 4. Synthesis of Compound 497. The coupling of compounds 490 (0.227 g, 0.540 mmol) and 496 (0.180 g, 0.360 mmol) was performed according to the method used to produce compound 433 in Example 9. Purification by flash chromatography (silica, 100:0 to 90:10 hexanes/ethyl acetate) afforded 497 (0.051 g, 18%) as a pale yellow oil.

Synthesis of Compound 344. The deprotection of compound 497 (0.051 g, 0.060 mmol) was performed according to the method used to produce compounds 326, 327 and 328 in Example 9, except for the omission of ammonium chloride, resulting in the formation of the corresponding carboxylic acid (not shown). The acid was re-esterified to the methyl ester 344 with TMS-diazomethane. Tetrabutylammonium fluoride (1.0 M in tetrahydrofuran, 5× molar excess) was added to a stirred solution of 497 in tetrahydrofuran (20 mL) at 0° C. and stirred for 5 h. The reaction was then poured into water (100 mL) and diluted with diethyl ether (150 mL). The aqueous layer was separated and extracted with diethyl ether (100 mL). The combined organic layers were then washed with brine (75 mL), dried over sodium sulfate, filtered and concentrated. The residue was redissolved in diethyl ether (20 mL), cooled to 0° C. and a solution of trimethylsilyl diazomethane (5× molar excess) was added and the reaction stirred for 20 min. The reaction mixture was then diluted with ethyl acetate and silica gel was added to quench the excess diazomethane. The reaction mixture was stirred for 10 min, then the insoluble material was removed by filtration and the filter cake was then washed with ethyl acetate and the filtrate concentrated. Purification by flash chromatography (silica, 100:0 to 95:5 methylene chloride/methanol) afforded a mixture of compound 344 and the corresponding lactone.

Synthesis of Compound 317. The mixture of compound 344 and the corresponding lactone from the previous step was hydrolyzed according to method used to produce Compounds 303, 304 and 305 in Example 9 to afford Compound 317 (0.014 g, 58%) as a light yellow film: ¹H NMR (500 MHz, CD₃OD) δ 7.01-6.96 (m, 2H), 6.95-6.90 (m, 2H), 6.56 (dd, J=15.4, 10.8 Hz, 1H), 6.44 (dd, J=16.0, 10.8 Hz, 1H), 5.90 (dd, J=15.2, 5.9 Hz, 1H), 5.70 (d, J=16.6 Hz, 1H), 4.65-4.61 (m, 1H), 4.50-4.46 (m, 1H), 3.94 (dd, J=9.6, 4.2 Hz, 1H), 3.87 (dd, J=9.7, 6.9 Hz, 1H), 2.36 (d, J=12.1 Hz, 1H), 2.08 (d, J=12.1 Hz, 1H), 2.06 (dd, J=14.6, 8.7 Hz, 1H), 1.71 (dd, J=14.6, 3.7 Hz, 1H), 1.07 (s, 6H).

### Example 25

### Synthesis of Compound 319 and 320

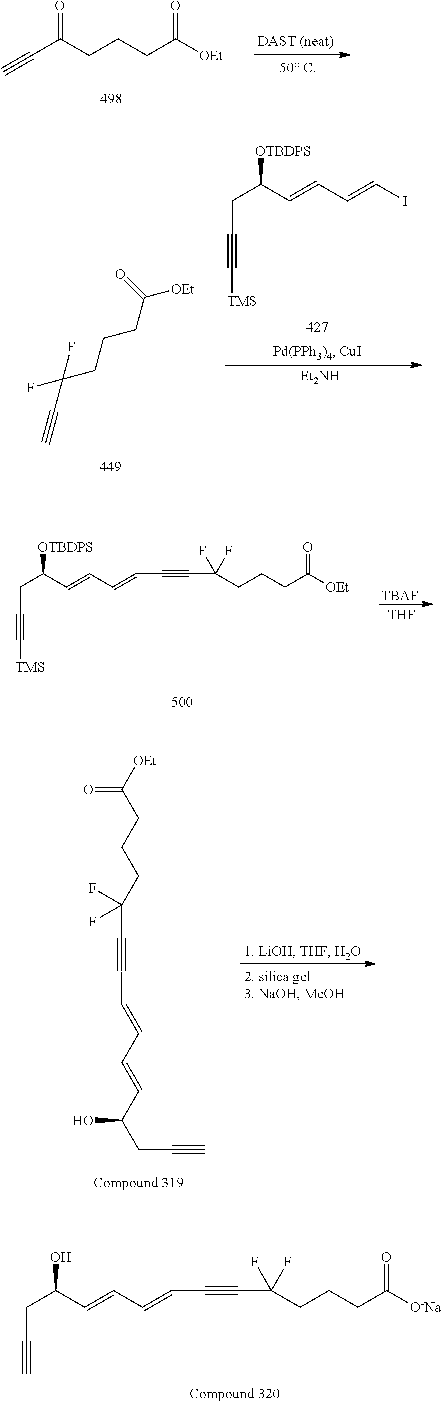

Synthesis of Compound 499. Compound 498 (2.00 g, 11.9 mmol) and DAST (3.80 g, 23.8 mmol) were combined in a polyethylene flask and heated at 50° C. for 3 h. The reaction mixture was then cooled to room temperature and then quenched by carefully pouring into a saturated aqueous sodium carbonate solution (50 mL) at 0° C. whilst maintaining vigorous stiffing. The mixture was then extracted with methylene chloride (2×50 mL). The combined organic layers were washed with brine (50 mL), dried over sodium sulfate, filtered and concentrated. Purification by flash chromatography (silica, 0 to 10% ether/pentane) afforded 499 (0.490 g, 22%) as a light yellow residue.

Synthesis of Compound 500. The coupling of 499 and 427 was performed according to method described for producing compound 409 in Example 2. Purification by flash chromatography (silica, 0 to 10% ethyl acetate/hexanes) afforded 500 in 65% yield.

Synthesis of Compound 319. The deprotection of 500 was performed according to method used to produce compounds 326, 327 and 328 in Example 9. Purification by flash chromatography (silica, 0 to 10% ethyl acetate/hexanes) afforded Compound 319 in 54% yield: ¹H NMR (500 MHz, CDCl₃) δ 6.74 (dd, J=15.7, 10.9 Hz, 1H), 6.38 (dd, J=15.4, 11.0 Hz, 1H), 5.95 (dd, J=15.2, 5.6 Hz, 1H), 5.65 (dt, J=15.6, 4.0 Hz, 1H), 4.40 (pentet, J=11.0 Hz, 1H), 4.15 (q, J=7.1 Hz, 2H), 2.53 (ddd, J=16.6, 5.4, 2.6 Hz, 1H), 2.46 (ddd, J=16.6, 6.5, 2.6 Hz, 1H), 2.39 (t, J=7.4 Hz, 2H), 2.02-2.17 (m, 4H), 1.94-1.87 (m, 2H), 1.27 (t, J=7.1, 3H); HPLC (Method 1) 95.5% (AUC).

Synthesis of Compound 320. The hydrolysis of Compound 319 was performed according to the method described for producing compounds 303, 304 and 305 in Example 9. Purification by flash chromatography (silica, 0 to 15% methanol/methylene chloride) afforded Compound 320 in 90% yield: ¹H NMR (500 MHz, CD₃OD) δ 6.80 (dd, J=15.6, 10.8 Hz, 1H), 6.41 (dd, J=15.4, 10.9 Hz, 1H), 6.05 (dd, J=15.2, 5.8 Hz, 1H), 5.75 (dt, J=15.6, 3.8 Hz, 1H), 4.29 (q, J=6.2 Hz, 1H), 2.43 (ddd, J=16.5, 6.0, 2.6 Hz, 1H), 2.39 (dd, J=6.8, 2.7 Hz, 1H), 2.37 (t, J=7.2 Hz, 3H), 2.30 (t, J=2.6 Hz, 1H), 2.16-2.06 (m, 2H), 1.87-1.81 (m, 2H); APCI MS, m/z 281 [M−H]⁻; HPLC (Method 1) 97.6% (AUC).

### Example 26

### Synthesis of Compound 321 and 345

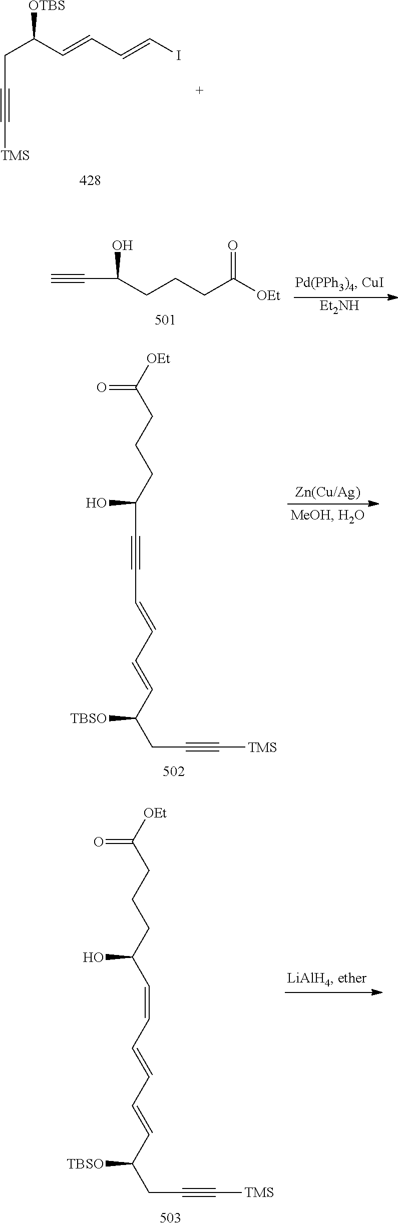

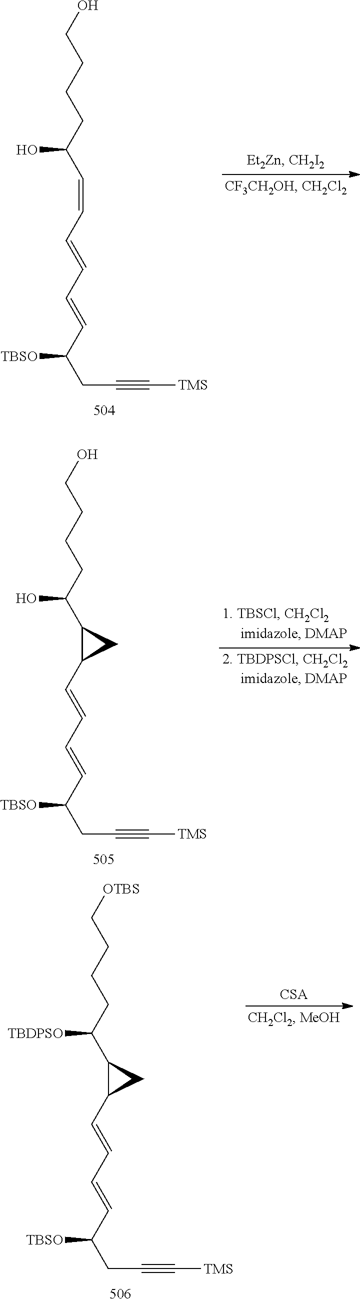

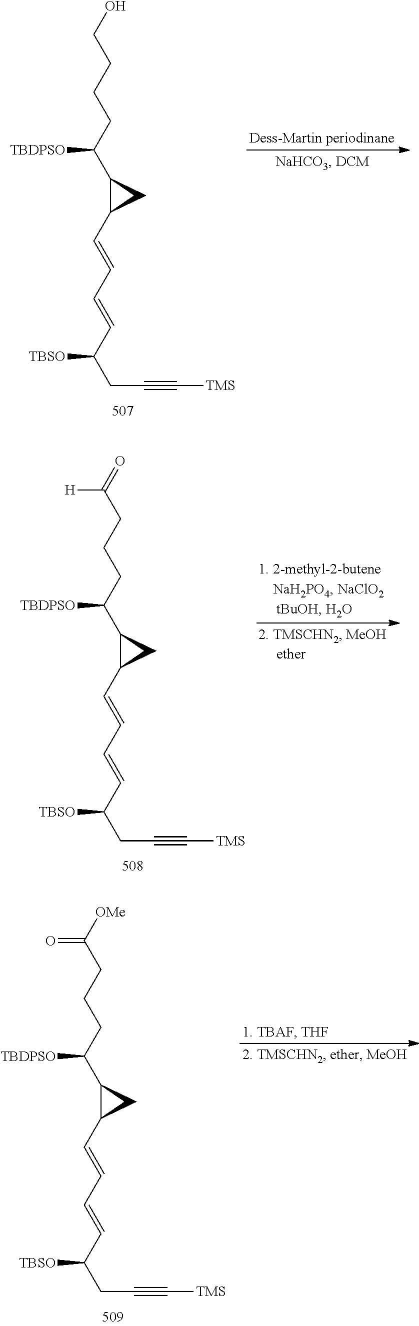

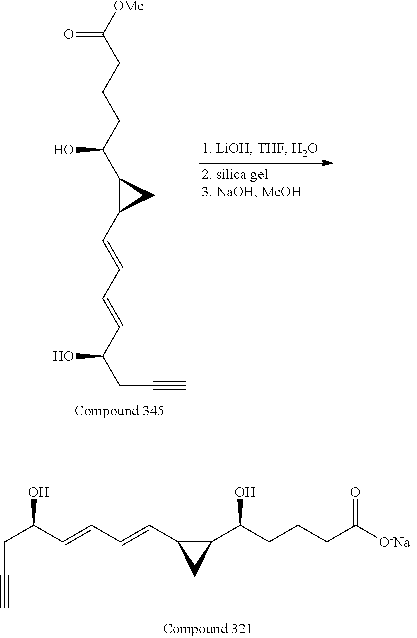

Synthesis of Compound 502. To a stirred solution of 428 in degassed diethylamine under argon, was added tetrakis(triphenylphosphino)palladium(0) (1% molar amount), followed by a solution of 501 in an equimolar amount in degassed diethylamine (25 mL). Copper(I) iodide (5% molar amount) was added and the reaction mixture stirred for 16 h at room temperature. The reaction mixture was then diluted with diethyl ether (350 mL) and washed with water (4×125 mL) and brine (2×100 mL), dried over sodium sulfate, filtered and concentrated. Purification by flash chromatography (silica, hexanes to 3:1 hexanes/ethyl acetate) afforded 502.

Synthesis of Compound 503. A solution of compound 502 (0.622 g, 1.30 mmol) in methanol (15 mL) was added to a stirred mixture of Zn(Cu/Ag) alloy (13.1 g) in degassed methanol/water (1:1, 50 mL) and the mixture was stirred at 40° C., under argon for 19 h. After this time, the reaction mixture was cooled to room temperature and filtered through diatomaceous earth. The filter cake was rinsed with ethyl acetate and the combined filtrates were concentrated to a predominantly aqueous residue which was then diluted with ethyl acetate. The organic layer was separated and washed with brine, dried over sodium sulfate, filtered and concentrated. Purification by silica plug filtration (silica, 7:3 hexanes/ethyl acetate) afforded 503 (0.509 g, 82%).

Synthesis of Compound 504. A solution of 503 (0.520 g, 1.08 mmol) in diethyl ether (15 mL) was cooled to 0° C. A solution of lithium aluminum hydride (1.4 mL, 1.0 M in diethyl ether, 1.4 mmol) was added dropwise and the reaction mixture was stirred at 0° C. for a further 2 h. The reaction was quenched by the addition of water (53 mL), 10% aqueous sodium hydroxide (53 mL) and water (159 mL) and then filtered through filter paper and the filtrate was concentrated. Purification by flash chromatography (silica, hexanes to 1:1 hexanes/ethyl acetate) afforded 504 (0.410 g, 86%).

Synthesis of Compound 505. A solution of trifluoroethanol (0.25 mL, 3.51 mmol) in methylene chloride (6 mL) was added drop-wise to a solution of diethyl zinc (3.51 mL, 1.0 M in hexanes, 3.51 mmol) in methylene chloride (6 mL) at 0° C. and stirred for a further 10 min. After this time, a solution of diiodomethane (0.28 mL, 3.51 mmol) in methylene chloride (6 mL) was added, followed by a solution of 504 in methylene chloride (6 mL). The reaction mixture was warmed to room temperature and stirred, with the exclusion of light, for 3 h. The reaction was quenched by the addition of a saturated aqueous ammonium chloride solution (10 mL) and then diluted with ethyl acetate (100 mL). The mixture was washed with saturated aqueous sodium bicarbonate solution (2×30 mL) and brine (30 mL), dried over sodium sulfate, filtered and concentrated. The crude material was combined with material from another batch of the same reaction (0.103 g scale) and purified by flash chromatography (silica, 5:2 to 2:3 hexanes/ethyl acetate) to afford 505 (0.320 g, 75%).

Synthesis of Compound 506. A mixture of 505 (0.320 g, 0.71 mmol), dimethylamino pyridine (0.004 g, 0.035 mmol) and triethylamine (0.13 g, 1.28 mmol) in methylene chloride (5 mL) was cooled to 0° C. tert-Butyldimethyl silyl chloride (0.107 g, 0.71 mmol) was added and the mixture was allowed to warm slowly to room temperature and then stirred for 16 h. After this time, the mixture was diluted with water (10 mL) and diethyl ether (30 mL). The organic layer was separated and washed with water (10 mL) and brine (10 mL), dried over sodium sulfate, filtered and concentrated. Purification by flash chromatography (silica, hexanes to 3:2 ethyl acetate/hexanes) afforded an intermediate (not shown; 0.347 g, 86%). The secondary alcohol on the intermediate was then protected as the tert-butyldiphenyl silyl ether according to method described for the production of compound 414 in Example 4 using tert-butylchlorodiphenylsilane instead of tert-butylchlorodimethylsilane. Purification by flash chromatography (silica, hexanes to 9:1 hexanes/ethyl acetate) afforded 506.

Synthesis of Compound 507. The primary tert-butyldimethyl silyl ether was deprotected according to method described for the production of compound 417 in Example 4. Purification by flash chromatography (silica, hexanes to 7:3 hexanes/ethyl acetate) afforded 507 in 51% yield.

Synthesis of Compound 508. A mixture of 507 (0.195 g, 0.283 mmol), sodium bicarbonate (0.356 g, 4.24 mmol) and Dess-Martin periodinane (0.240 g, 0.566 mmol) in methylene chloride (3 mL) was stirred at room temperature for 2.5 h. The reaction mixture was quenched with 10% aqueous sodium thiosulfate (5 mL) and diluted with diethyl ether (10 mL). The organic layer was separated and washed with saturated aqueous ammonium chloride (5 mL) and brine (5 mL), dried over sodium sulfate, filtered and concentrated. Purification by flash chromatography (silica, hexanes to 7:3 hexanes/ethyl acetate) afforded 508 (0.126 g, 65%).

Synthesis of Compound 509. A solution of 508 (0.126 g, 0.183 mmol) in tert-butanol/water (3:1, 5 mL) was cooled to 0° C. Sodium phosphate (0.025 g, 0.183 mmol) and 2-methyl-2-butene (0.064 g, 0.916 mmol) were added, followed by sodium chlorite (0.033 g, 0.366 mmol) and the mixture was then stirred at 0° C. for 1 h. The reaction mixture was then poured into a slurry of silica gel and diethyl ether/methylene chloride (10 mL) and stirred for 10 min and then filtered through a sintered glass funnel. The filter cake was washed with ethyl acetate and the combined filtrates were concentrated. The residue was diluted with ethyl acetate (40 mL) and washed with water (20 mL) and brine (2×20 mL), dried over sodium sulfate, filtered and concentrated. At this stage the residue was combined with another batch of crude carboxylic acid (0.013 g) and dissolved in diethyl ether/methanol (4:1, 5 mL) and cooled to 0° C. Trimethylsilyl diazomethane (0.91 mL, 2.0 M in diethyl ether, 1.83 mmol) was added and the reaction mixture was stirred at 0° C. for 1 h. The reaction mixture was poured into a slurry of silica gel and ethyl acetate and the mixture was stirred until gas evolution had ceased. The mixture was filtered, the filter cake was rinsed with ethyl acetate and the combined filtrates concentrated. Purification by flash chromatography (silica, hexanes to 7:3 hexanes/ethyl acetate) afforded 509 (0.117 g, 89%).

Synthesis of Compound 345. The deprotection of compound 509 was performed according to the method described for the production of compounds 326, 327 and 328 in Example 9. Purification by flash chromatography (silica, 4:1 to 1:1 hexanes/ethyl acetate) afforded Compound 345 in 53% yield: ¹H NMR (500 MHz, CD₃OD) δ 6.25 (dd, J=14.5, 10.3 Hz, 1H), 6.15 (dd, J=14.8, 10.5 Hz, 1H), 5.64 (dd, J=15.0, 6.5 Hz, 1H), 5.44 (dd, J=14.8, 9.1 Hz, 1H), 4.20 (q, J=6.4 Hz, 1H), 3.64 (s, 3H), 3.18-3.11 (m, 1H), 2.40 (ddd, J=16.8, 6.3, 2.9 Hz, 1H), 2.34 (ddd, J=16.6, 6.6, 2.7 Hz, 1H), 2.30 (dt, J=7.3, 3.9 Hz, 2H), 2.26 (t, J=2.6 Hz, 1H), 1.80-1.70 (m, 1H), 1.69-1.60 (m, 2H), 1.55-1.48 (m, 2H), 1.13-1.05 (m, 1H), 1.00 (dt, J=8.1, 4.7 Hz, 1H), 0.61-0.56 (m, 1H).

Synthesis of Compound 321. The hydrolysis of Compound 345 was performed according to the method for producing Compounds 303, 304 and 305 in Example 9 and afforded Compound 321 in 90% yield: ¹H NMR (500 MHz, CD₃OD) δ 6.25 (dd, J=14.4, 10.2 Hz, 1H), 6.15 (dd, J=10.5, 14.9 Hz, 1H), 5.64 (dd, J=15.0, 6.5 Hz, 1H), 5.46 (dd, J=14.8, 9.0 Hz, 1H), 4.20 (q, J=6.3 Hz, 1H), 3.18-3.12 (m, 1H), 2.40 (ddd, J=16.5, 6.0, 2.6 Hz, 1H), 2.34 (ddd, J=16.5, 6.5, 2.7 Hz, 1H), 2.28-2.20 (m, 3H), 1.79-1.69 (m, 1H), 1.69-1.58 (m, 2H), 1.58-1.50 (m, 2H), 1.14-1.06 (m, 1H), 1.00 (dt, J=8.1, 4.7 Hz, 1H), 0.62-0.57 m, 1H); ESI MS m/z 277 [M−H]⁻; HPLC (Method 1) 95.4% (AUC).

### Example 27

### Synthesis of Compound 322 and 346

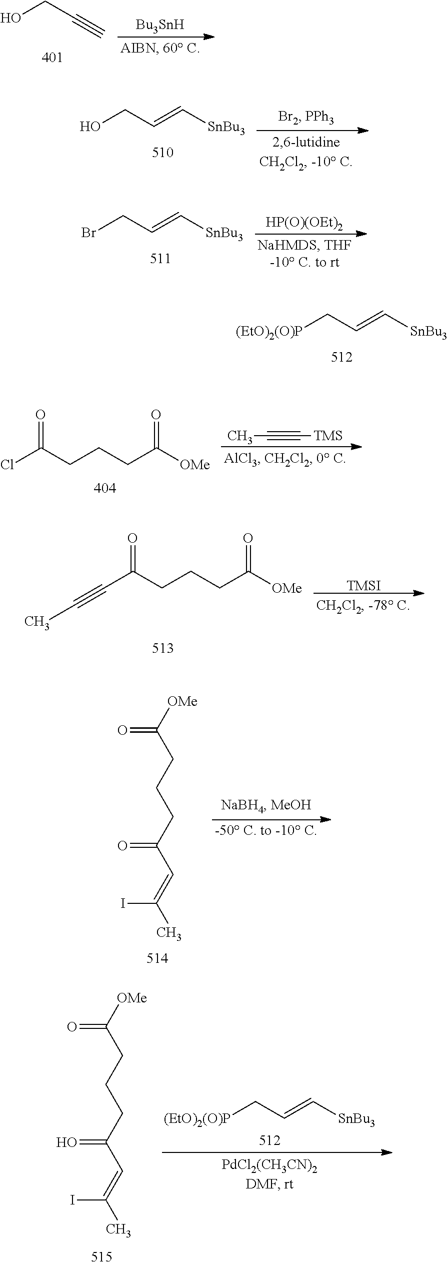

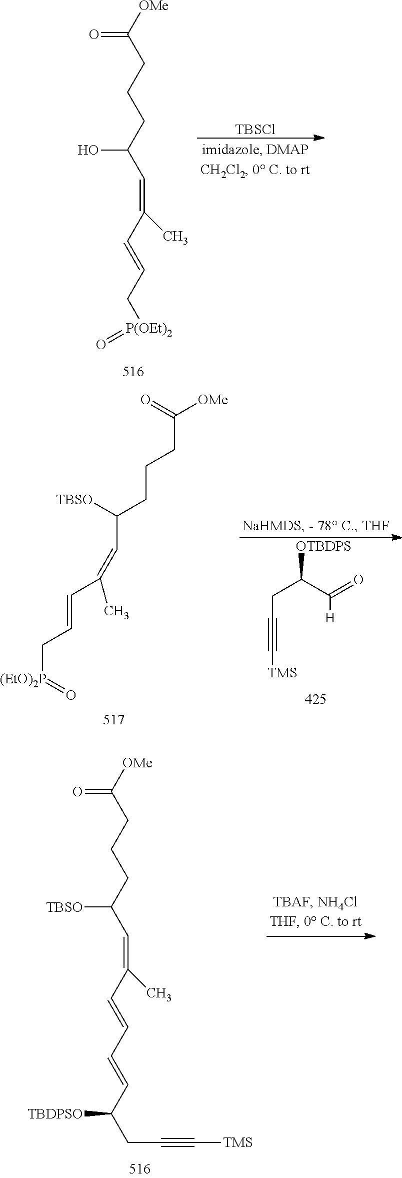

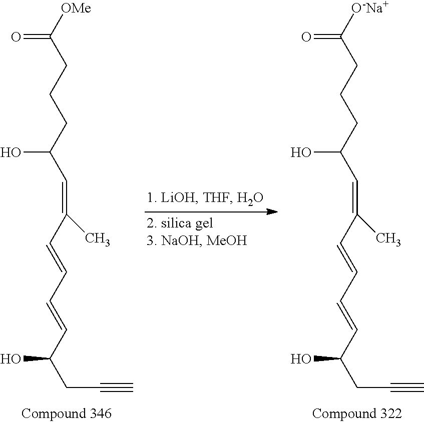

Synthesis of Compound 510. AIBN (100 mg, 0.61 mmol) was added to a mixture of propargyl alcohol 401 (2.50 g, 44.6 mmol) and tributyltin hydride (16.9 g, 58.0 mmol) at room temperature. The reaction was heated at 60° C. for 24 h then cooled to room temperature. Purification by flash chromatography (silica, 7-15% ethyl acetate/hexanes) afforded 510 (7.15 g, 46% yield) as a clear, colorless oil.

Synthesis of Compound 511. A mixture of bromine (4.94 g, 30.9 mmol) and triphenylphosphine (8.10 g, 30.9 mmol) in methylene chloride (94 mL) at −10° C. was stirred for 30 minutes. A solution of compound 510 and 2,6-lutidine (6.61 g, 61.8 mmol) in methylene chloride (69 mL) was then added. The reaction was stirred at −10° C. for 50 minutes and then diluted with hexanes (300 mL) and filtered through a plug of silica gel (90:10 hexanes/ethyl acetate) and concentrated. Purification by flash chromatography (silica, 0.5% ethyl acetate/hexanes) afforded 511 (7.93 g, 94%) as a clear, colorless oil.

Synthesis of Compound 512. Sodium bis(trimethylsilyl)amide (27.5 mL, 1.0 M in tetrahydrofuran, 27.5 mmol) was added slowly to a solution of diethyl phosphite (3.80 g, 27.5 mmol) in tetrahydrofuran (62 mL) at −78° C. After stirring for 30 minutes, compound 511 (10.2 g, 25.0 mmol) in tetrahydrofuran (42 mL) was added drop-wise. The reaction was stirred for 2.5 h at −78° C. then allowed to warm to −20° C. over 1 h and then stirred at room temperature for 45 minutes. The reaction was quenched with water (75 mL) and brine (75 mL) and extracted with diethyl ether (3×200 mL). The combined organic layers were dried over sodium sulfate, filtered and concentrated. Purification by flash chromatography (silica, 35-50% ethyl acetate/hexanes) afforded 512 (7.38 g, 63%) as a clear, colorless oil.

Synthesis of Compound 513. Trimethylaluminum chloride (0.967 g, 7.24 mmol) was added portion-wise to a mixture of methyl 5-chloro-5-oxovalerate 404 (1.19 g, 7.24 mmol) and trimethyl(prop-1-ynyl)silane (0.813 g, 7.24 mmol) in methylene chloride (36 mL) at 0° C. The mixture was stirred at 0° C. for 3 h then poured into a mixture of dilute HCl and ice. The aqueous portion was extracted with diethyl ether (3×100 mL) and the combined organic layers were washed with water, saturated sodium bicarbonate, and brine then dried with sodium sulfate, filtered and concentrated. Purification by flash chromatography (silica, 10-15% ethyl acetate/hexanes) afforded 513 (0.803 g, 66%) as a clear, colorless oil.

Synthesis of Compound 514. A mixture of compound 513 (0.200 g, 1.19 mmol) and trimethylsilyl iodide (0.238 g, 1.19 mmol) in methylene chloride (4 mL) was stirred at −78° C. for 5 minutes. After this time the reaction was quenched by the addition of diethyl ether and water (1:1, 2 mL) and then allowed to slowly warm to room temperature. The mixture was diluted with methylene chloride (40 mL) and washed with saturated aqueous sodium bicarbonate (15 mL), 10% aqueous sodium bisulfite (15 mL), and brine (15 mL), dried over sodium sulfate, filtered, and concentrated. Purification by flash chromatography (silica, 7.5-12% ethyl acetate/hexanes) afforded 514 (84 mg, 24% yield) as a clear, colorless oil.

Synthesis of Compound 515. Sodium borohydride (1.07 g, 28.1 mmol) was added to a solution of compound 514 (2.08 g, 7.03 mmol) in methanol (23 mL) at −50° C. The reaction was stirred at −50° C. for 3 h and then allowed to warm to −20° C. over 1 hour. The reaction was stirred for a further 30 minutes between −20 and −10° C. and then quenched with brine (250 mL) and warmed to room temperature. The mixture was then extracted with ethyl acetate (3×200 mL) and the combined organic layers were washed with brine (150 mL), dried over sodium sulfate, filtered and concentrated. Purification by flash chromatography (silica, 25% ethyl acetate/hexanes) afforded 515 (1.87 g, 90%) as a clear, colorless oil.

Synthesis of Compound 516. A solution of compound 515 (1.87 g, 6.30 mmol) in dimethylformamide (30 mL) was degassed with argon for 15 minutes. Dichlorobis(triphenylphosphine) palladium (0.163 g, 0.63 mmol) was then added followed by a solution of compound 512 (5.86 g, 12.6 mmol) in dimethylformamide (10 mL) and the mixture stirred for 18 h. After this time ethyl acetate (400 mL) was added and the mixture was washed with brine (3×200 mL), dried over sodium sulfate, filtered, and concentrated. Purification by flash chromatography (6-8% methanol/methylene chloride) afforded 516 (2.82 g, >100% yield) as a yellow oil.

Synthesis of Compound 517. tert-Butylchlorodimethylsilane (1.14 g, 7.56 mmol) was added to a solution of compound 516 (2.19 g, 6.30 mmol), imidazole (0.640 g, 9.45 mmol), and dimethylaminopyridine (0.079 g, 0.63 mmol) in methylene chloride (32 mL) at 0° C. The reaction was allowed to slowly warm to room temperature and then stirred overnight. After this time, saturated aqueous ammonium chloride (200 mL) was added and the mixture was then extracted with ethyl acetate (3×150 mL). The combined organic layers were dried over sodium sulfate, filtered and concentrated. Purification by flash chromatography (silica, 50-70% ethyl acetate/hexanes) afforded 517 (2.19 g, 75% yield for 2 steps) as a clear, yellow oil.

Synthesis of Compound 516. Sodium hexamethyldisilazide (0.46 mL, 1.0 M solution in tetrahydrofuran, 0.46 mmol) was added drop-wise over 1 minute to a solution of 517 (195 mg, 0.42 mmol) in tetrahydrofuran (3 mL) at −78° C. The reaction mixture was stirred at −78° C. for 5 minutes and then 425 (260 mg, 0.63 mmol) in tetrahydrofuran (1 mL) was added. The reaction was stirred at −78° C. for 2 hours and then at −45° C. for 1 h and then quenched with saturated aqueous ammonium chloride (20 mL) and allowed to warm to room temperature. The mixture was extracted with ethyl acetate (3×100 mL) and the combined organic layers were dried over sodium sulfate, filtered and concentrated. Purification by flash chromatography (silica, 5% ethyl acetate/hexanes) afforded 516 (140 mg, 47%) as a clear, colorless oil which was a 1:1 mixture of diastereomers at the C-5 position.

Synthesis of Compound 346. The desilylation of compound 516 was performed according to the method used to produce compounds 326, 327, and 328 in Example 9. Purification by flash chromatography (silica, 90:10 to 50:50 hexanes/ethyl acetate) afforded Compound 346 in 37% yield: ¹H NMR (500 MHz, CD₃OD) δ 6.67 (d, J=14.6 Hz, 1H), 6.39 (dd, J=14.5, 10.6 Hz, 1H), 6.33 (dd, J=14.6, 10.6 Hz, 1H), 5.86 (dd, J=14.3, 6.3 Hz, 1H), 5.32 (d, J=8.9 Hz, 1H), 4.60-4.53 (m, 1H), 4.26 (q, J=6.3 Hz, 1H), 3.67 (s, 3H), 2.43 (ddd, J=16.6, 6.0, 2.6 Hz, 1H), 2.38 (ddd, J=16.5, 6.7, 2.7 Hz, 1H), 2.34 (t, J=6.8 Hz, 2H), 2.29-2.26 (m, 1H), 1.88 (s, 3H), 1.72-1.56 (m, 3H), 1.49-1.38 (m, 1H).

Synthesis of Compound 322. The hydrolysis of compound 346 was performed according to the method for producing compounds 303, 304 and 305 in Example 9 to afford Compound 322 in 71% yield: ¹H NMR (500 MHz, CD₃OD) δ 6.70 (d, J=15.1 Hz, 1H), 6.39 (dd, J=15.0, 10.5 Hz, 1H), 6.32 (dd, J=15.0, 10.5 Hz, 1H), 5.84 (dd, J=14.7, 6.5 Hz, 1H), 5.34 (d, J=8.9 Hz, 1H), 4.61-4.55 (m, 1H), 4.25 (q, J=6.0 Hz, 1H), 2.43 (ddd, J=16.5, 5.9, 2.6 Hz, 1H), 2.37 (ddd, J=16.5, 6.7, 2.7 Hz, 1H), 2.29-2.26 (m, 1H), 2.17 (t, J=7.0 Hz, 2H), 1.88 (s, 3H), 1.69-1.53 (m, 3H), 1.50-1.40 (m, 1H); ESI MS m/z=277 [M−H]⁻; HPLC (Method 1) 95.1% (AUC).

### Example 28

### Synthesis of Compound 324 and 325

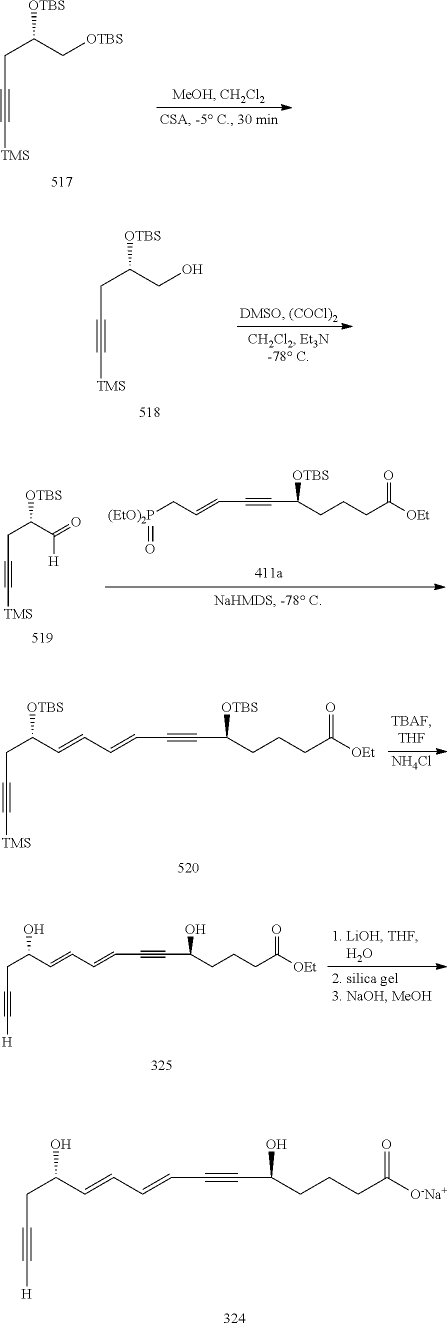

Synthesis of Compound 518. The deprotection of compound 517 was performed in an analogous manner to the scheme for producing Compound 417 according to Example 4. Purification by flash chromatography (silica, hexanes to 95:5 hexanes/ethyl acetate) afforded 518 in 47% yield: ¹H NMR (500 MHz, CDCl₃) δ 3.84-3.85 (m, 1H), 3.67 (ddd, J=11.1, 5.8, 3.8 Hz, 1H), 3.58 (ddd, J=11.6, 7.2, 4.8 Hz, 1H), 2.45 (dd, J=16.8, 7.2 Hz, 1H), 2.40 (dd, J=16.8, 6.0 Hz, 1H), 1.88 (dd, J=7.0, 6.0 Hz, 1H), 0.90 (s, 9H), 0.14 (s, 9H), 0.13 (s, 3H), 0.11 (s, 3H); ¹³C NMR (125 MHz, CDCl₃) δ 103.3, 86.8, 71.5, 65.9, 25.8, 25.4, 18.0, −4.5, −4.7.

Synthesis of Compound 519. The oxidation of compound 518 was performed in an analogous manner to the scheme for producing Compound 418 according to Example 4. Purification by flash chromatography (silica, hexanes to 9:1 hexanes/ethyl acetate) afforded 519 in 85% yield: ¹H NMR (500 MHz, CDCl₃) δ 9.63 (d, J=1.1 Hz, 1H), 4.11 (ddd, J=7.8, 5.1, 1.1 Hz, 1H), 2.64 (dd, J=15.1, 5.0 Hz, 1H), 2.48 (dd, J=17.0, 7.9 Hz, 1H), 0.94 (s, 9H), 0.15 (s, 9H and 3H), 0.14 (s, 3H); ¹³C NMR (125 MHz, CDCl₃) δ 202.1, 101.8, 87.4, 76.1, 25.89, 25.7, 24.5, 18.2, −0.1, −4.7.

Synthesis of Compound 520. The coupling of compounds 519 and 411a was performed according to the method for producing compound 433 in Example 9. Purification by flash chromatography (silica, hexanes to 85:15 hexanes/ethyl acetate) afforded 520 in 35% yield: ¹H NMR (500 MHz, CDCl₃) δ 6.52 (dd, J=15.5, 10.9 Hz, 1H), 6.23 (dd, J=15.2, 10.9 Hz, 1H), 5.81 (dd, J=15.2, 5.8 Hz, 1H), 5.59 (d, J=15.6 Hz, 1H), 4.50 (td, J=6.2, 1.5 Hz, 1H), 4.32 (q, J=5.9 Hz, 1H), 4.13 (q, J=7.1 Hz, 2H), 2.44 (dd, J=16.6, 6.7 Hz, 1H), 2.40-2.28 (m, 3H), 1.86-1.64 (m, 4H), 1.25 (t, J=7.1 Hz, 3H), 0.90 (s, 9H), 0.90 (s, 9H), 0.14 (s, 9H), 0.13 (s, 3H), 0.11 (s, 3H), 0.09 (s, 3H), 0.05 (s, 3H); ¹³C NMR (125 MHz, CDCl₃) δ 173.5, 140.8, 137.8, 129.0, 110.9, 103.7, 93.2, 86.5, 83.4, 63.1, 60.2, 37.9, 33.9, 29.9, 25.8, 20.8, 18.2, 14.2, 0.0, −4.5, −4.6, −4.8, −5.1.

Synthesis of Compound 325. The desilylation of compound 520 was performed in an analogous manner to the scheme for producing Compound 335 according to Example 22. Purification by flash chromatography (silica, hexanes to 3:2 hexanes/ethyl acetate) afforded 325 in 86% yield: ¹H NMR (500 MHz, CDCl₃) δ 6.57 (dd, J=15.6, 10.9 Hz, 1H), 6.34 (dd, J=15.3, 11.0 Hz, 1H), 5.86 (dd, J=15.3, 5.9 Hz, 1H), 5.65 (d, J=15.6 Hz, 1H), 4.58-4.48 (m, 1H), 4.37 (pentet, J=5.3 Hz, 1H), 4.14 (q, J=7.1 Hz, 2H), 2.51 (ddd, J=16.6, 5.4, 2.6 Hz, 1H), 2.45 (ddd, J=16.7, 6.5, 2.9 Hz, 1H), 2.37 (t, J=6.7 Hz, 2H), 2.08 (t, J=2.6 Hz, 1H), 2.05 (d, J=4.9 Hz, 1H), 1.93 (d, J=5.4 Hz, 1H) 1.88-1.68 (m, 4H), 1.26 (t, J=7.1 Hz, 3H); ¹³C NMR (125 MHz, CDCl₃) δ 173.5, 141.0, 136.2, 130.2, 111.5, 92.6, 84.0, 79.9, 71.3, 70.0, 62.6, 60.4, 37.1, 33.8, 27.6, 20.6, 14.3.

Synthesis of Compound 324. The hydrolysis of compound 325 was performed in an analogous manner to the scheme for producing Compound 323 according to Example 22 and afforded 324 in 93% yield: ¹H NMR (500 MHz, CD₃OD) δ 6.46 (dd, J=15.5, 10.8 Hz, 1H), 6.24 (dd, J=15.3, 10.9 Hz, 1H), 5.78 (dd, J=15.2, 6.1 Hz, 1H), 5.58 (d, J=16.4 Hz, 1H), 4.38-4.29 (m, 1H), 4.15 (q, J=6.4 Hz, 1H), 2.32 (ddd, J=16.6, 6.0, 2.6 Hz, 1H), 2.26 (ddd, J=16.6, 6.7, 2.7 Hz, 1H), 2.18 (t, J=2.7 Hz, 1H), 2.09 (t, J=7.1 Hz, 1H), 1.70-1.53 (m, 4H). APCI MS m/z 261 [M−H]⁻. HPLC (Method 1) 99.0% (AUC).

### Example 29

### Rat Collagen-induced Arthritis (rCIA) Model

Two-month-old, female Lewis rats (Charles River Labs) were anesthetized with 3-5% isoflurane and arthritis was induced by intradermal injection of 0.3 ml (100 μl at three different sites) of an emulsion containing 1.5 mg/ml of bovine type II collagen (CII) (Elastin Products Co. Inc. Owensville, Mo.) in Incomplete Freund's Adjuvant (IFA) (Difco Laboratories, Detroit, Mich.) on day 0 at the base of the tail using a 30 gauge needle. A second injection was given on day 6. Control rats were injected with an equal amount of IFA only.

Test compounds of Formula I are formulated by diluting concentrated ethanol stock solutions with phosphate buffered saline (PBS) and dosed by oral gavage at 0.3 mg/kg twice a day (BID). Dosing was initiated on day 8 and continued to the end of the study, generally day 19 or day 20. Control animals were administered PBS alone.

The volume of both hind paws were measured using a water displacement plethysmometer (Ugo Basile, Biological Research Apparatus, Italy), and the onset of arthritis was indicated by increased paw volume, which appears approximately on day 11 post injection. Both paw volumes and body weights are measured throughout the study every 2-3 days. Nineteen to twenty-one days after CII injection, the rats are euthanized with CO₂. Blood is collected by cardiac puncture and serum is saved. Both hind ankles are collected in 10% buffered formalin and saved (they can later be processed for histology).

Percent inhibition values were measured as follows. First, the magnitude of disease effect was calculated by taking the difference in paw swelling between control animals and animals that had not been administered CII (“untreated effect”). Then, the difference is paw swelling between the compound-treated animals and the animals that had not been administered CII was determined (“treated effect”). The difference between the untreated effect and the treated effect is then divided by the treated effect and multiplied by 100 to arrive at percent inhibition. Percent inhibition reported was from day of “peak” or maximal disease (manifested by maximum paw swelling), typically day 17. The results for various compounds of the invention are shown in the table below.

TABLE 2
Activity of Compounds in rCIA Model.

| Compound No. | % inhibition |
|---|---|
| 301 | 11 |
| 302 | 37 |
| 303 | 0 |
| 304 | 28 |
| 314 | 27 |
| 315 | 7 |
| 318 | 5 |
| 320 | 18 |
| 321 | 10 |
| 323 | 38 |

### Example 30

### Murine Delayed-type-Hypersensitivity (DTH) Model

Balb/c mice were sensitized by application of 20 μl of 0.5% of DNFB dissolved in acetone/olive oil to their footpads on day 0 and day 1. On day 5 animals are dosed intravenously with 0.3 mg/kg of test compound (or a negative control). Fifteen minutes later, 10 μl of 0.8% of DNFB dissolved in acetone/olive oil is topically applied externally on the right ear of the animal. As a control, the left ear is treated the same way with vehicle alone (acetone/olive oil).

The compounds (controls and test) are administered once daily until the completion of the study.

Within 24 to 48 hours after ear challenge, animals are anesthetized with isoflurane and the ear swelling is measured using a micrometer. The micrometer calipers are closed around the top portion of the each external ear until resistance from the ear is felt. Usually, the peak of the ear swelling happens around 48 hours after challenge.

Percent inhibition is determined in a similar manner as described for the CII model, above.

The results for various compounds of the invention are shown in the table below.

TABLE 3
Activity of Compounds in Murine DTH Model.

| Compound No. | % inhibition |
|---|---|
| 301 | 15 |
| 302 | 24 |
| 303 | 21 |
| 304 | 20 |
| 306 | 16 |
| 314 | 28 |
| 315 | 32 |
| 318 | 11 |
| 320 | 35 |
| 321 | 32 |
| 323 | 46 |

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A compound of the formula I, 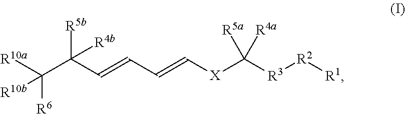
or a pharmaceutically acceptable salt thereof, wherein:
X is selected from —C≡C—, —C(R⁷)═C(R⁷)—, -(cyclopropyl)-, -(cyclobutyl)-, -(cyclopentyl)-, and -(cyclohexyl)-;
R¹ is selected from —OR^{a}, —N(R^{a})—SO₂—R^{c} and —N(R^{a})(R^{b}), wherein each of R^{a} and R^{b} is independently selected from H, C₁-C₆-alkyl, aryl, aralkyl, heteroaryl, and heteroaralkyl, and R^{c} is selected from C₁-C₆-alkyl, aryl, aralkyl, heteroaryl, and heteroaralkyl;
R² is selected from —CH₂—, —C(O)—, —SO₂—, —PO(OR)—, and tetrazole;
R is selected from hydrogen and alkyl;
R³ is selected from a carbocyclic ring, a heterocyclic ring, —(CH₂)ₙ—, CH₂C(O)CH₂, and —CH₂—O—CH₂, wherein:
n is an integer from 1 to 3;
any hydrogen atom in R³ is optionally and independently replaced by halo, (C₁-C₅)-alkyl, perfluoroalkyl, aryl, heteroaryl, hydroxy, or O—(C₁-C₅)-alkyl; and
any two hydrogen atoms bound to a common carbon atom in R³ are optionally taken together with the carbon atom to which they are bound to form a carbocyclic or heterocyclic ring;
each of R^{4a} and R^{4b} is independently selected from hydrogen, halo, —OH, —O—(C₁-C₅)-alkyl, —O-aryl, O-heteroaryl, —O—C(O)—(C₁-C₅)-alkyl, —O—C(O)-aryl, —O—C(O)-heteroaryl, —O—C(O)—O—(C₁-C₅)-alkyl, —O—C(O)—O-aryl, —O—C(O)—O-heteroaryl, and —O—C(O)—N(R^{a})(R^{b}), wherein any alkyl, aryl or heteroaryl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester, thioacyl, thioether, amino, amido, acylamino, cyano, and nitro;
each of R^{5a} and R^{5b} is independently selected from hydrogen, halo, (C₁-C₅)-alkyl, perfluoroalkyl, aryl, and heteroaryl;
R⁶ is selected from -phenyl, —(C₁-C₅)-alkyl, —(C₃-C₇)-cycloalkyl, —C≡C-phenyl, —C≡C—(C₃-C₇)-cycloalkyl, —C≡C—(C₁-C₅)-alkyl, and —O-phenyl, wherein phenyl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester, thioacyl, thioether, amino, amido, acylamino, cyano, and nitro, and R⁶ is additionally selected from —C≡CH when:
a) X is —C(R⁷)═C(R⁷)— or -(cyclopropyl)-; or
b) each of R^{4a} and R^{4b} is hydrogen or halo; or
c) each of R^{5a} and R^{5b} is halo; or
d) R² is —CH₂—;
each R⁷ is independently selected from hydrogen and (C₁-C₅)-alkyl, or two occurrences of R⁷ may optionally be taken together with the carbons to which they are attached to form a 5- or 6-membered ring;
each of R^{10a} and R^{10b} is independently selected from hydrogen, (C₁-C₅)-alkyl, perfluoroalkyl, O—(C₁-C₅)-alkyl, aryl and heteroaryl, or R^{10a} and R^{10b} are taken together with the carbon atom to which they are bound to form a carbocyclic or heterocyclic ring;
and each double bond is independently in an E- or a Z-configuration.

2. The compound of claim 1, wherein X is —C≡C—.

3. The compound of claim 1, wherein R^{4b} is hydrogen.

4. The compound of claim 1, wherein R^{4a} is hydrogen.

5. The compound of claim 1, wherein R^{4a} is fluoro; and R^{5a} is fluoro.

6. The compound of claim 1, wherein R^{4b} is fluoro; and R^{5b} is fluoro.

7. The compound of claim 1, wherein each of R^{4a} and R^{4b} is independently selected from —OH, —O—(C₁-C₅)-alkyl, O-aryl, O-heteroaryl, —O—C(O)—(C₁-C₅)-alkyl, O—C(O)-aryl, O—C(O)-heteroaryl, and —O—C(O)—N(R^{a})(R^{b}).

8. The compound of claim 1, wherein R² is —CH₂—.

9. The compound of claim 1, wherein X is -(cyclopropyl)-.

10. The compound of claim 1, wherein X is —C(R⁷)═C(R⁷)—.

11. The compound of claim 1, wherein:
each of R^{a} and R^{b} is independently selected from H and C₁-C₆-alkyl;
R^{c} is C₁-C₆-alkyl;
R³ is selected from —(CH₂)ₙ— and —CH₂—O—CH₂, wherein n is an integer from 1 to 3; and up to two hydrogen atoms in R³ are optionally and independently replaced by (C₁-C₅)-alkyl;
each of R^{4a} and R^{4b} is independently selected from hydrogen, halo, —OH, —O—(C₁-C₅)-alkyl; and
each of R^{10a} and R^{10b} is hydrogen.

12. The compound of claim 1, wherein R^{4a} is in an (S) configuration

13. The compound of claim 1, wherein R^{4b} is in an (R) configuration.

14. The compound of claim 1, wherein each double bond is in an E-configuration.

15. The compound of claim 1, selected from any one of:

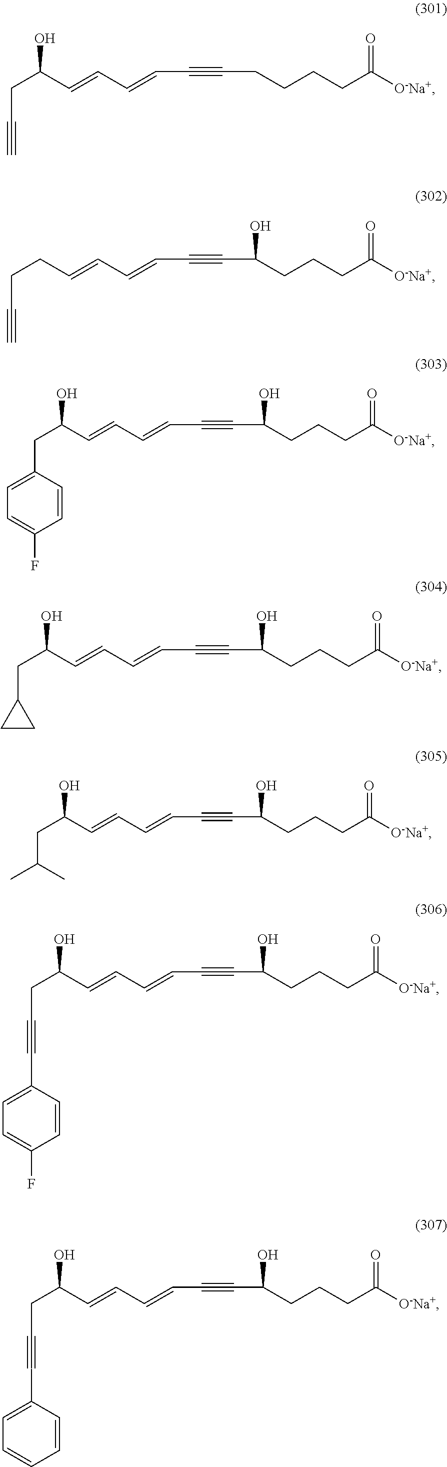

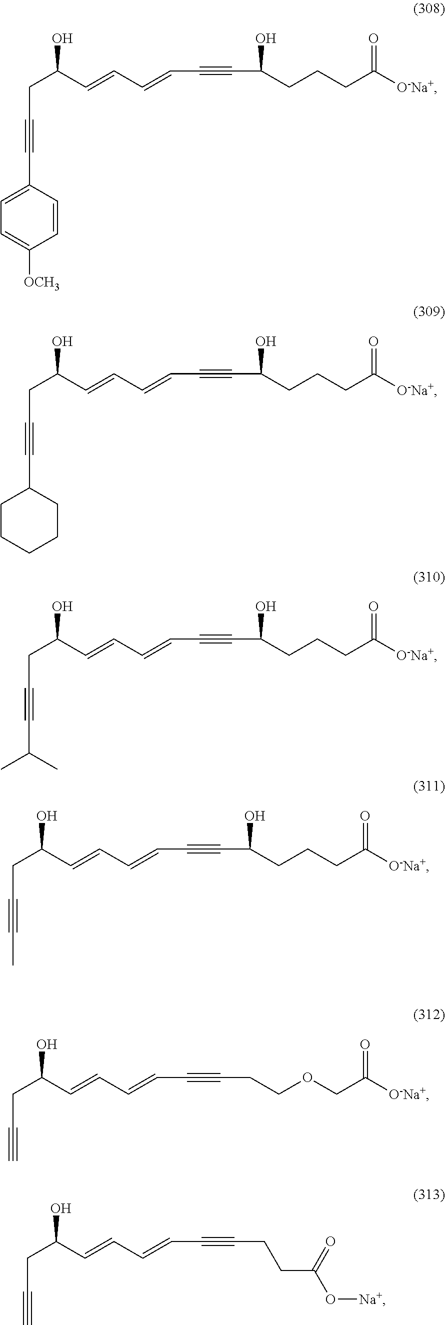

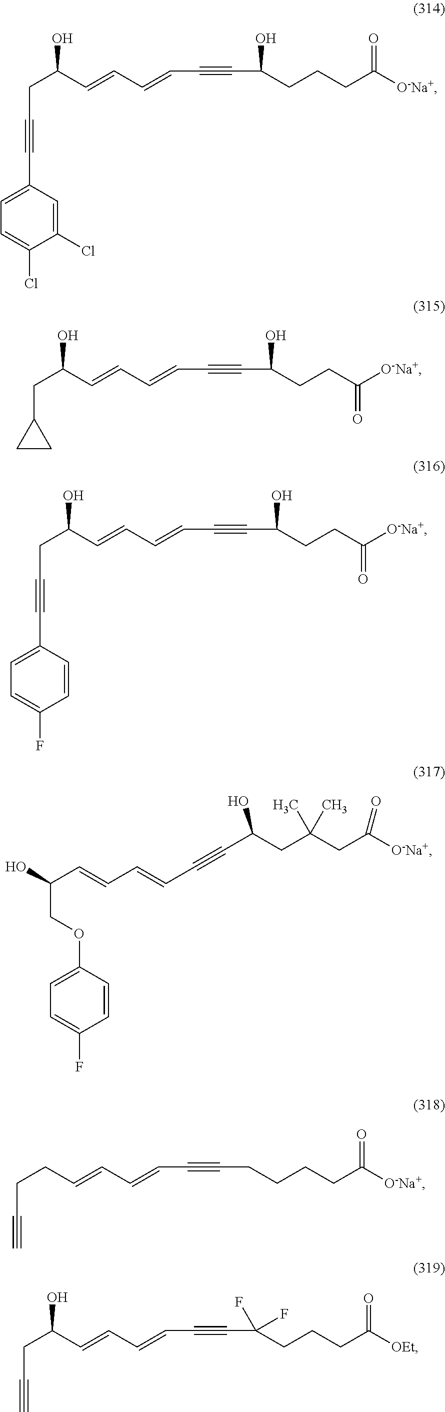

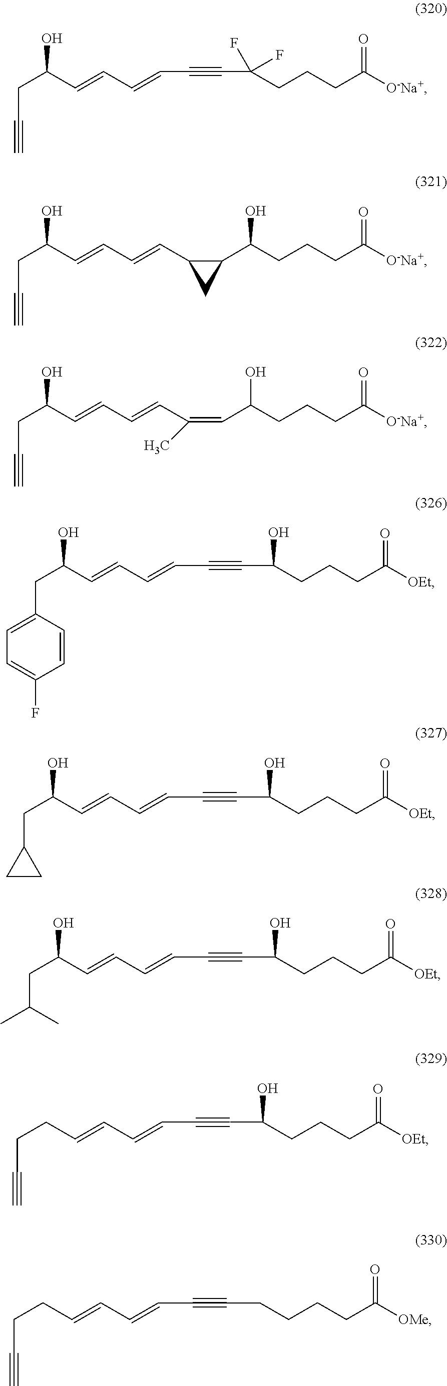

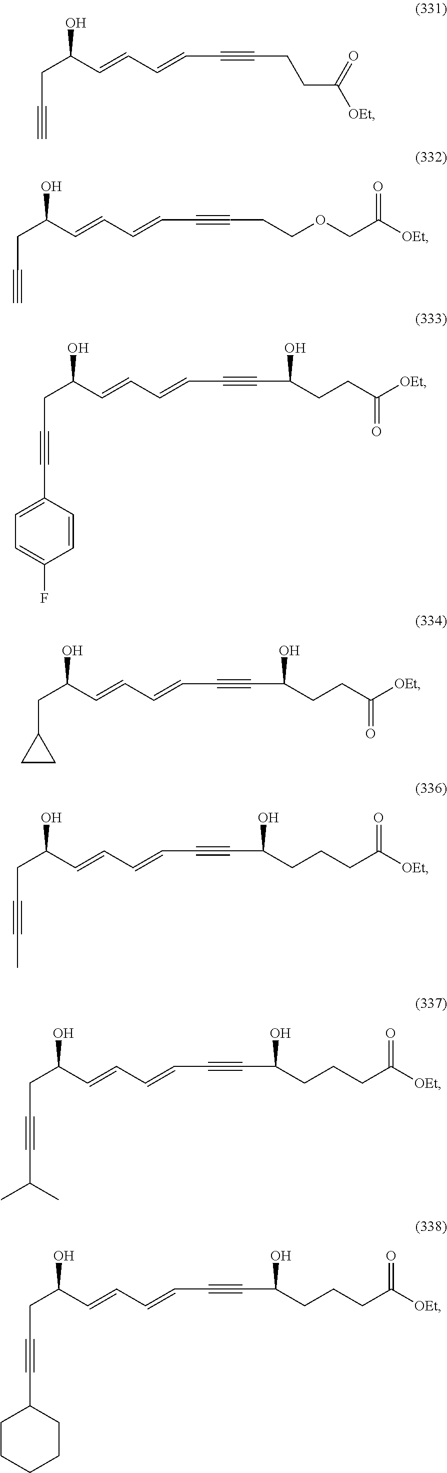

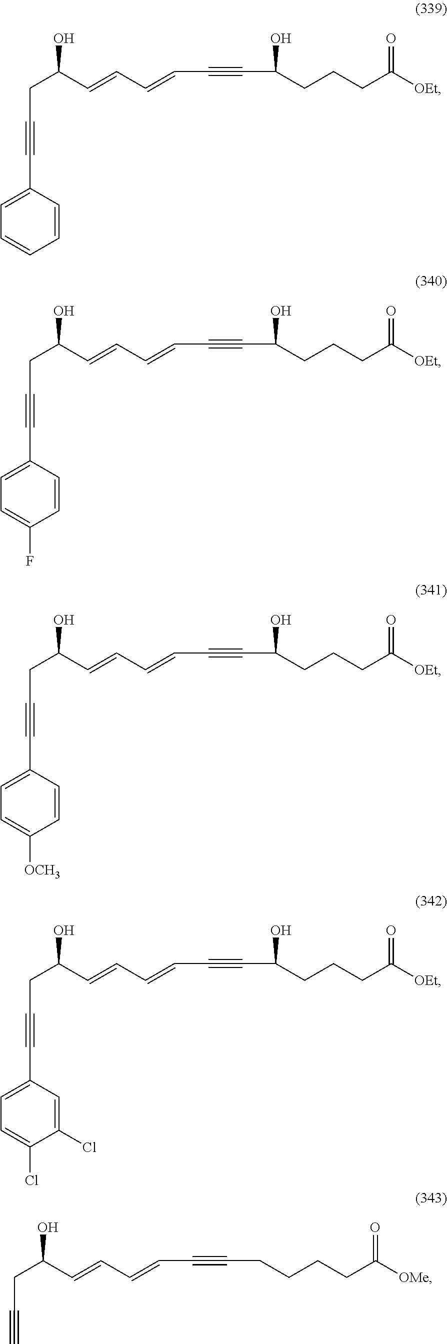

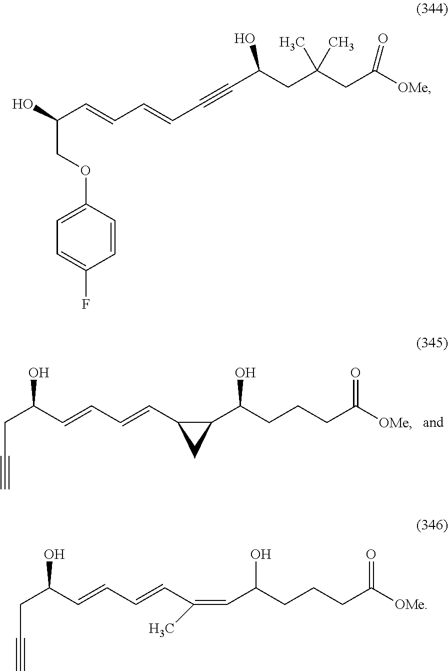

16. A compound of the formula II, 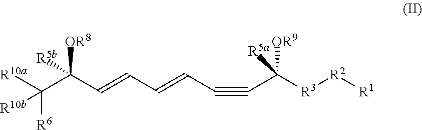
or a pharmaceutically acceptable salt thereof, wherein:
R¹ is selected from —OR^{a}, —N(R^{a})—SO₂—R^{c} and —N(R^{a})(R^{b}), wherein each of R^{a} and R^{b} is independently selected from H, C₁-C₆-alkyl, aryl, aralkyl, heteroaryl, and heteroaralkyl, and R^{c} is selected from C₁-C₆-alkyl, aryl, aralkyl, heteroaryl, and heteroaralkyl;
R² is selected from —C(O)—, —SO₂—, —PO(OR)—, and tetrazole;
R is selected from hydrogen and alkyl;
R³ is selected from —(CH₂)ₙ— and —CH₂—O—CH₂, wherein n is an integer from 1 to 3; and optionally up to two hydrogen atoms in R³ are independently replaced by halo, (C₁-C₅)-alkyl, or O—(C₁-C₅)-alkyl;
each of R^{5a} and R^{5b} is independently selected from hydrogen, (C₁-C₅)-alkyl, perfluoroalkyl, aryl, and heteroaryl;
R⁶ is selected from —C≡CH, -phenyl, —(C₁-C₅)-alkyl, —(C₃-C₇)-cycloalkyl, —C≡C-phenyl, —C≡C—(C₃-C₇)-cycloalkyl, —C≡C—(C₁-C₅)-alkyl, and —O-phenyl, wherein phenyl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester, thioacyl, thioether, amino, amido, acylamino, cyano, and nitro;
each of R⁸ and R⁹ are independently selected from hydrogen, —(C₁-C₅)-alkyl, -aryl, -heteroaryl, —C(O)—(C₁-C₅)-alkyl, —C(O)-aryl, —C(O)-heteroaryl, —C(O)—O—(C₁-C₅)-alkyl, —C(O)—O-aryl, —C(O)—O-heteroaryl, and —C(O)—N(R^{a})(R^{b}), wherein any alkyl, aryl or heteroaryl is optionally substituted with up to 3 substituents independently selected from halo, (C₁-C₅)-alkyl, O—(C₁-C₅)-alkyl, hydroxyl, carboxyl, ester, alkoxycarbonyl, acyl, thioester, thioacyl, thioether, amino, amido, acylamino, cyano, and nitro;
each of R^{10a} and R^{10b} is independently selected from hydrogen, (C₁-C₅)-alkyl, perfluoroalkyl, O—(C₁-C₅)-alkyl, aryl and heteroaryl, or
R^{10a} and R^{10b} are taken together with the carbon atom to which they are bound to form a carbocyclic or heterocyclic ring; and
wherein each double bond is independently in an E- or a Z-configuration.

17. The compound of claim 16 selected from any one of: [chemical structure]
